(19) United States
(12) Patent Application Publication
(10) Pub. No.: US 2022/0117958 A1
(43) Pub. Date: Apr. 21, 2022

(54) COMPOSITIONS AND METHODS FOR INHIBITING ACSS2

(71) Applicant: THE TRUSTEES OF THE UNIVERSITY OF PENNSYLVANIA, Philadelphia, PA (US)

(72) Inventors: Shelley L. Berger, Wayne, PA (US); Philipp Mews, New York, NY (US); Jeffrey Winkler, Wynnewood, PA (US); Andrew Glass, Philadelphia, PA (US); Gabor Egervari, Philadelphia, PA (US)

(21) Appl. No.: 17/277,759

(22) PCT Filed: Sep. 26, 2019
(86) PCT No.: PCT/US19/53108

§ 371 Date: Mar. 19, 2021

Related U.S. Application Data
(60) Provisional application No. 62/736,638, filed on Sep. 26, 2018.
(60) Provisional application No. 62/824,092, filed on Mar. 26, 2019.

(51) Int. Cl. A61K 31/498; A61P 25/30

(52) U.S. Cl. CPC A61K 31/498; A61P 25/30

(57) ABSTRACT

The present invention provides compositions and methods for inhibiting ACSS2 for modulating histone acetylation or for treating or preventing a neurological disease or disorder.

### CROSS-REFERENCE TO RELATED APPLICATIONS

The present application claims priority to U.S. Provisional Application Ser. No. 62/736,638, filed Sep. 26, 2018 and 62/824,092, filed on Mar. 26, 2019, each of which is incorporated by reference herein in its entirety.

### STATEMENT REGARDING FEDERALLY SPONSORED RESEARCH OR DEVELOPMENT

This invention was made with government support under Grant No. P01AG031862 awarded by The National Institutes for Health. The government has certain rights in the invention.

### BACKGROUND OF THE INVENTION

Memory formation involves synaptic restructuring and requires the coordinated expression of neuronal genes through poorly understood processes that modify chromatin (Kandel, E. R. et al., 2014, Cell, 157:163-186; Zovkic, I. B. et al., 2013, Learn. Mem., 20:61-74). Histone acetylation is a key regulator of memory storage and restructures chromatin in distinct brain regions that have been implicated in learning and memory, most prominently in the hippocampus (Graff, J. et al., 2013, Nat. Rev. Neurosci., 14:97-111). Hippocampal memory consolidation requires the transcription factor CREB and the coactivator CREB binding protein (CBP), specifically the histone acetyltransferase (HAT) activity of CBP (Wood, M. A. et al., 2005, Learn. Mem., 12:111-119; Korzus, E. et al., 2004, Neuron, 42:961-972). Furthermore, inhibitors of histone deacetylases enhance memory consolidation (Graff, J. et al., 2013, Nat. Rev. Neurosci., 14:97-111). However, the mechanisms that regulate neuronal histone acetylation in long-term memory remain incompletely understood.

Direct sensing of intermediary metabolites by chromatin-modifying enzymes such as acetyltransferases can dynamically adapt chromatin structure and gene expression (Kaelin, W. G. Jr. et al., 2013, Cell, 153:56-69; Katada, S., et al., 2012, Cell, 148:24-28). Alteration of pools of intracellular acetyl-CoA manipulates histone acetylation (Cai, L., et al., 2011, Mol. Cell, 42:426-437; Wellen, K. E. et al., 2009, Science, 324:1076-1080); thus, metabolic enzymes that generate nuclear acetyl-CoA might directly control histone acetylation and gene expression (Gut, P. et al., 2013, Nature, 502:489-498; Pietrocola, F. et al., 2015, Cell Metab., 21:805-821). In mammalian cells, there are two principal enzymes that generate acetyl-CoA for histone acetylation:acetate-dependent acetyl-CoA synthetase 2 (ACSS2) and citrate-dependent ATP-citrate lyase (ACL) (Pietrocola, F. et al., 2015, Cell Metab., 21:805-821). The relative importance of ACSS2 and ACL for nuclear histone acetylation differs by tissue type, developmental state, and disease (Wellen, K. E. et al., 2009, Science, 324:1076-1080; Pietrocola, F. et al., 2015, Cell Metab., 21:805-821), but the roles of these enzymes in post-mitotic neuronal cells are unknown.

Addictive disorders are complex conditions that manifest from compulsive substance use despite harmful consequences. Often those affected experience distorted thinking, behaviors and body functions in response to the craving. In one example, alcohol use disorder (AUD) is characterized by craving, loss of control over alcohol intake and continued use despite negative consequences. It affects a large segment of the population in the United States and worldwide and continues to impose a tremendous burden on society in the form of associated health concerns, loss of workforce and crime, which is further exacerbated by the chronic, relapsing pattern of the disease. Effective therapeutic options for AUD remain scarce and mostly rely on counseling, behavioral treatment and mutual support groups. In fact, only three pharmaceutical medications are currently approved by the U.S. Food and Drug Administration for the treatment of AUD—naltrexone, acomprosate and disulfiram. However, low efficacy and lack of compliance due to adverse side effects severely limit the therapeutic potential of these drugs. As such, there remains a critical and immediate need for a better understanding of the neurobiological underpinnings of AUD, which could drive translational research and inform future therapeutic interventions.

Cocaine use disorder (CUD) is characterized by craving, loss of control over cocaine intake and continued use despite negative consequences. It affects a large segment of the population in the United States and worldwide and continues to impose a tremendous burden on society in the form of associated health concerns, loss of workforce and crime, which is further exacerbated by the chronic, relapsing pattern of the disease.

Effective therapeutic options remain scarce and mostly rely on counseling, behavioral treatment and mutual support groups. Currently available options include cognitive-behavioral therapy, contingency management or motivational incentives-providing rewards to patients who remain substance free, therapeutic communities-drug-free residences in which people in recovery from substance use disorders help each other to understand and change their behaviors, and community based recovery groups, such as 12-step programs. Strikingly, there are still no FDA-approved pharmacological tools to treat CUD, emphasizing an important unmet need in this field.

Thus, there remains a need in the art for therapies to treat neurological, cognitive diseases and disorders, including PTSD, and addictive disorders such as CUD and AUD.

### SUMMARY OF THE INVENTION

The invention also provides method for treating or preventing a neurological and cognitive disease or disorder. In one embodiment, the method comprises administering a composition comprising a compound of Formula (l) to a subject in need thereof:

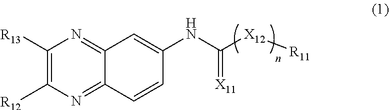

wherein, X₁₁ is selected from the group consisting of C(R₁₄)(R₁₅), O, S and NR₁₅;

each occurrence of X₁₂ is selected from the group consisting of C(R₁₄)(R₁₅), O, S and NR₁₅;

R₁₁ is selected from the group consisting of —C₁-C₂₅ alkyl, cycloalkyl, heterocyclyl, aryl, heteroaryl, and combinations thereof, wherein R₁₁ is optionally substituted; R₁₂ and R₁₃ are each independently selected from the group consisting of hydrogen, —C₁-C₆ alkyl, —C₃-C₆ aryl, and —C₄-C₆ heteroaryl, wherein R₁₂ and R₁₃ are optionally substituted;

each occurrence of R₁₄ and R₁₅ is independently selected from the group consisting of hydrogen, halogen, —OH, and C₁-C₆ alkyl; and

n is an integer from 0-4.

In one embodiment, the method comprises administering a composition comprising a compound of Formula (2) to a subject in need thereof:

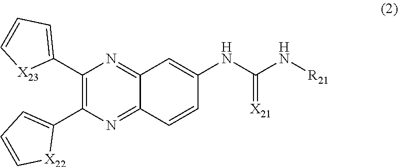

wherein, X₂₁ is O, or S;

X₂₂ and X₂₃ are each independently selected from the group consisting of NR₂₂, O, and S; and

R₂₁ is selected from the group consisting of —C₁-C₂₅ alkyl, cycloalkyl, heterocyclyl, aryl, heteroaryl, and combinations thereof, wherein R₁₁ is optionally substituted; and

each occurrence of R₂₂ is independently selected from the group consisting of hydrogen and C₁-C₆ alkyl.

In one embodiment, the method comprises administering a composition comprising a compound of Formula (3) to a subject in need thereof:

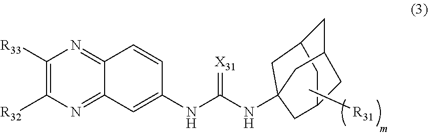

wherein, X₃₁ is selected from the group consisting of C(R₃₄)(R₃₅), O, S and NR₃₅;

each R₃₁ is independently hydrogen, —C₁-C₁₀ alkyl, halogen, —OH, or ═O or ═S formed by joining two R₃₁s,

R₃₂ and R₃₃ are each independently selected from the group consisting of hydrogen, —C₁-C₆ alkyl, —C₃-C₆ aryl, and —C₄-C₆ heteroaryl, wherein R₁₂ and R₁₃ are optionally substituted;

each occurrence of R₃₄ and R₃₅ is independently selected from the group consisting of hydrogen, halogen, —OH, and C₁-C₆ alkyl; and

m is an integer from 0-15.

In one embodiment, the method comprises administering a composition comprising a compound selected from the group consisting of

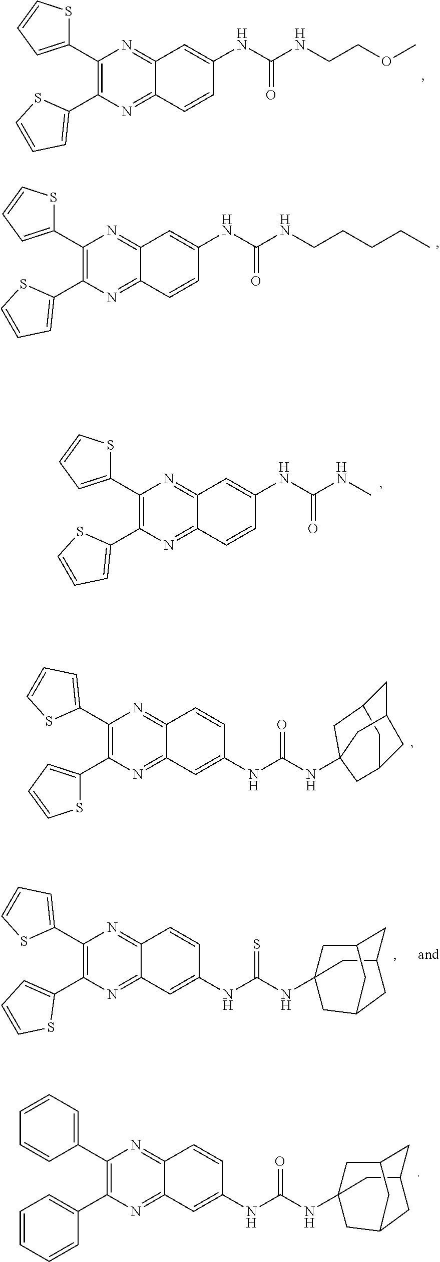

In one embodiment, the neurological and cognitive disease or disorder is selected from the group consisting of post-traumatic stress disorder (PTSD), depression, addiction or addiction-related disease or disorder, anxiety disorder, panic disorders, obsessive-compulsive disorder, and phobias. In one embodiment, the neurological and cognitive disease or disorder is PTSD. In one embodiment, the addiction is alcoholism or cocaine addiction. In one embodiment, the addiction-related disease or disorder is acute and/or chronic alcohol induced memory deficit.

In one embodiment, the invention provides a method for treating or preventing a neurological and cognitive disease or disorder in a subject in need thereof. In one embodiment, the method comprises (a) treating the subject with the compound of claim 9 during trauma recall and memory reconsolidation; and (b) subsequently treating the subject with cognitive behavioral therapy.

In one embodiment, the step treating the subject with the compound of claim 9 during trauma recall and memory reconsolidation is repeated up to 12 times. In one embodiment, the step treating the subject with the compound of claim 9 during trauma recall and memory reconsolidation is repeated 2, 3, 4, 5 or 6 times.

In one embodiment, the cognitive behavioral therapy is Cognitive Behavioral Therapies (CBT), Prolonged Exposure (PE), Cognitive Processing Therapy (CPT), or Eye Movement Desensitization and Reprocessing (EMDR). In one embodiment, the cognitive behavioral therapy is cognitive processing therapy.

In one embodiment, the invention provides a compound according to Formula (1):

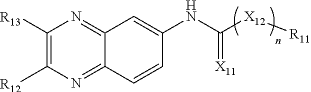

wherein, X₁₁ is selected from the group consisting of C(R₁₄)(R₁₅), O, S and NR₁₅; each occurrence of X₁₂ is selected from the group consisting of C(R₁₄)(R₁₅), O, S and NR₁₅; R₁₁ is selected from the group consisting of —C₁-C₂₅ alkyl, cycloalkyl, heterocyclyl, aryl, heteroaryl, and combinations thereof, wherein R₁₁ is optionally substituted; R₁₂ and R₁₁ are each independently selected from the group consisting of hydrogen, —C₁-C₆ alkyl, —C₃-C₆ aryl, and —C₄-C₆ heteroaryl, wherein R₁₂ and R₁₃ are optionally substituted; each occurrence of R₁₄ and R₁₅ is independently selected from the group consisting of hydrogen, halogen, —OH, and C₁-C₆ alkyl; and n is an integer from 0-4.

In one embodiment the invention provides a compound according to Formula (1):

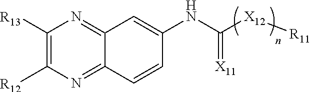

wherein, X₁₁ is selected from the group consisting of C(R₁₄)(R₁₅), O, S and NR₁₅; each occurrence of XI is selected from the group consisting of C(R₁₄)(R₁₅), S and NR₁₅; R₁₁ is selected from the group consisting of —C₁-C₂₅ alkyl, cycloalkyl, heterocyclyl, aryl, heteroaryl, and combinations thereof, wherein R₁₁ is optionally substituted; R₁₂ and R₁₃ are each independently selected from the group consisting of hydrogen, —C₁-C₆ alkyl, —C₃-C₆ aryl, and —C₄-C₆ heteroaryl, wherein R₁₂ and R₁₃ are optionally substituted; each occurrence of R₁₄ and R₁₅ is independently selected from the group consisting of hydrogen, halogen, —OH, and C₁-C₆ alkyl; and n is an integer from 0-4.

In one embodiment, the compound according to Formula (1) is a compound according to Formula (2)

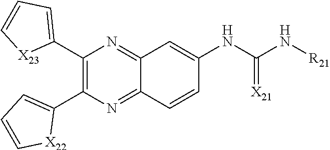

wherein, X₂₁ is O, or S; X₂₂ and X₂₃ are each independently selected from the group consisting of NR₂₂, O, and S; and R₂₁ is selected from the group consisting of —C₁-C₂₅ alkyl, cycloalkyl, heterocyclyl, aryl, heteroaryl, and combinations thereof, wherein R₁₁ is optionally substituted; and each occurrence of R₂₂ is independently selected from the group consisting of hydrogen and C₁-C₆ alkyl.

In one embodiment, the compound according to Formula (1) is a compound according to Formula (3)

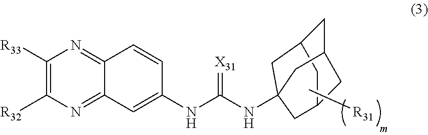

wherein, X₃₁ is selected from the group consisting of C(R₃₄)(R₃₅), O, S and NR₃₅; each R₃₁ is independently hydrogen, —C₁-C₁₀ alkyl, halogen, —OH, or ═O or ═S formed by joining two R₃₁s, R₃₂ and R₃₃ are each independently selected from the group consisting of hydrogen, —C₁-C₆ alkyl, —C₃-C₆ aryl, and —C₄-C₆ heteroaryl, wherein R₁₂ and R₁₃ may be optionally substituted; each occurrence of R₃₄ and R₃₅ is independently selected from the group consisting of hydrogen, halogen, —OH, and C₁-C₆ alkyl; and m is an integer from 0-15.

In one embodiment, the compound is selected from the group consisting of

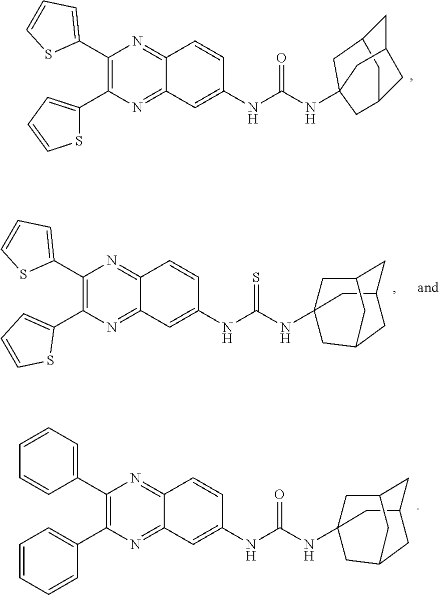 [SMILES: O=C(Nc1ccc2nc(-c3cccs3)c(-c3cccs3)nc2c1)NC12CC3CC(CC(C3)C1)C2 ; S=C(Nc1ccc2nc(-c3cccs3)c(-c3cccs3)nc2c1)NC12CC3CC(CC(C3)C1)C2 ; O=C(Nc1ccc2nc(-c3ccccc3)c(-c3ccccc3)nc2c1)NC12CC3CC(CC(C3)C1)C2]

### BRIEF DESCRIPTION OF THE DRAWINGS

The following detailed description of embodiments of the invention will be better understood when read in conjunction with the appended drawings. It should be understood that the invention is not limited to the precise arrangements and instrumentalities of the embodiments shown in the drawings.

FIG. 1 depicts assay design to determine efficacy to reduce catalytic ACSS2 activity and histone H3 lysine 9 acetylation in vitro—Ntera2 cells were maintained in DMEM (Gibco) with 10% FBS and GlutaMAX (Gibco). Cells were treated for 24 hours with 5 mM sodium acetate in the absence of glucose and compound ADG-204, ADG-205, ADG-206, or vehicle (DMSO). Cells were lysed in RIPA buffer containing 50 mM Tris pH 8.0, 0.5 mM EDTA, 150 mM NaCl, 1% NP40, 1% SDS, supplemented with protease inhibitor cocktail (Life Technologies, number 78446) and 10 mM sodium butyrate. Protein concentration was determined by BCA protein assay (Life Technologies, number 23227), and equal amounts of protein were directly loaded onto polyacrylamide gels. Proteins were separated on 4-12% Bis-Tris polyacrylamide gels (NuPAGE). After transfer to nitrocellulose membrane, 3% BSA in TBS supplemented with 0.1% Tween 20 (TBST) was used to block the membrane at room temperature for 1 h. Primary antibodies were diluted in TBST and incubated at 4° C. overnight. The antibodies used were anti-H3 (Abcam ab1791), anti-H3K9ac (Abcam ab4441), anti-GAPDH (Fitzgerald Industries 10R-G109A). The membrane was washed three times with TBST, each for 10 min, followed by incubation with HRP-conjugated secondary antibodies at room temperature for 1 h, in TBST. The membrane was washed again three times and imaged with a Fujifilm LAS-4000 imager.

FIG. 2 depicts the chemical structure and activity of ADG-204.

FIG. 3 depicts the chemical structure and activity of ADG-205.

FIG. 4 depicts the chemical structure and activity of ADG-206.

FIG. 5 depicts the brain availability for ADG-204, ADG-205, ADG-206, and ADG-207.

FIG. 6 depicts the pharmacokinetics of ADG I-204 in rats.

FIG. 7 depicts the pharmacokinetics of ADG I-205 in rats.

FIG. 8 depicts the pharmacokinetics of ADG I-206 in rats.

FIG. 9 depicts the pharmacokinetics of ADG I-207 in rats.

FIG. 10, comprising FIG. 10A through FIG. 10H, depicts experimental results.

FIG. 10A depicts relative abundance of deuterated histone acetylation in dorsal Hippocampus (dHPC), ventral Hippocampus (vHPC), Cortex, Liver, and Muscle at 8 hours after i.p. injection of d6-EtOH. FIG. 10B depicts relative abundance of deuterated histone acetylation in dorsal Hippocampus (dHPC), ventral Hippocampus (vHPC), Cortex, Liver, and Muscle at 24 hours after i.p. injection of d6-EtOH. FIG. 10C depicts C13-EtOH (carbon 1 heavy labeled) introduced via intraperitoneal injection readily labels hippocampal histone acetylation (% increase over natural abundance of 13C acetyl groups in saline-injected animals, n=1). FIG. 10D depicts that, in contrast to heavy d6-EtOH, non-labeled EtOH control does not increase the natural abundance of heavy histone acetylation in the hippocampus. FIG. 10E depicts histone acetylation is relatively independent of liver alcohol metabolism in skeletal muscle. Relative abundance of deuterated histone acetylation in skeletal muscle tissue at 30 minutes and 4 hours in WT mice, and 30 minutes in hippocampal ACSS2 KD mice. FIG. 10F depicts heavy acetate introduced via intraperitoneal injection readily labels histone acetylation in the dorsal hippocampus (n=2 at 30 min, n=3 per group at other time points; data are mean±s.e.m.). FIG. 10G depicts heavy acetate introduced via intraperitoneal injection readily labels histone acetylation in the cortex (n=2 at 30 min, n=3 per group at other time points; data are mean±s.e.m.). FIG. 10H depicts acetate levels measured mass spec in hippocampal tissue following acetate and ethanol injections (n=3 per group; data are mean±s.e.m., two-tailed unpaired T test, 30 min Acetate vs. Saline, P=0.0335; two-tailed unpaired T test, 30 min EtOH vs. saline, P=0.0285).

FIG. 11, comprising FIG. 11A through FIG. 11F, depicts mass spec quantification of metabolite labeling in hippocampal tissue at 30 minutes following i.p. d6-EtOH injection. FIG. 11A depicts experimental results demonstrating d6-EtOH label was incorporated into hippocampal acetate pools. FIG. 11B depicts experimental results demonstrating d6-EtOH did not contribute to glucose pool. FIG. 11C depicts experimental results demonstrating d6-EtOH only minimally contribute to lactate. FIG. 11D depicts experimental results demonstrating d6-EtOH did not contribute to hydroxybutyrate in hippocampus. FIG. 11E depicts experimental results demonstrating labeling of 3-Hydroxybutyrate was not observed, in contrast to hippocampal Glutamine pools. FIG. 11F depicts experimental results demonstrating labeling of 3-Hydroxybutyrate was not observed, in contrast to hippocampal Isocitrate/Citrate pools.

FIG. 12, comprising FIG. 12A through FIG. 12G, depicts experimental results demonstrating mass spectrometry analysis of d6-EtOH in dHPC ACSS2 KD. FIG. 12A depicts knockdown of ACSS2 expression in dorsal hippocampus prevents incorporation of the heavy label into histone acetylation. FIG. 12B depicts in the same animal, incorporation of the heavy label in the ventral hippocampus (where ACSS2 levels are normal) is not changed when compared to control mice. FIG. 12C depicts, ChIP-seq for H3K9ac and H3K27ac in untreated and EtOH-treated WT and ACSS2 KD animals (n=3 independent replicates). Genome-browser track view shows the FstlI gene locus (Chr16: 37,776,000-37,793,000).

FIG. 12D depicts ChIP-seq for H3K9ac in vivo shows increased acetylation genome-wide following EtOH injection (339/458 H3K9ac peaks; called with MACS2, 10% FDR threshold DiffBind; box-and-whisker plots show the first and third quartile values and the median (center) value with whiskers extending to 1.5× the interquartile range; two-sided Mann-Whitney rank-sum test, P<2.2E-16). FIG. 12E depicts ChIP-seq for H3K27ac in vivo shows increased acetylation genome-wide following EtOH injection (490/816 H3K27ac peaks; called with MACS2, 10% FDR threshold DiffBind; box-and-whisker plots show the first and third quartile values and the median (center) value with whiskers extending to 1.5× the interquartile range; two-sided Mann-Whitney rank-sum test, P=8.42e-11). FIG. 12F depicts induction of H3K9ac is diminished in ACSS2 KD (458 H3K9ac peaks; box-and-whisker plots show median value with whiskers extending to 1.5× the interquartile range; two-sided Mann-Whitney rank-sum test. P-value <2.2E-16). FIG. 12G depicts induction of H3K27ac is diminished in ACSS2 KD (458 H3K9ac peaks, 816 H3K27ac peaks; box-and-whisker plots show median value with whiskers extending to 1.5× the interquartile range; two-sided Mann-Whitney rank-sum test, P=2.22e-6).

FIG. 13, comprising FIG. 13A through FIG. 13C, depicts experimental data demonstrating ChIP-seq for H3K9ac and H3K27ac in untreated and EtOH-treated WT and ACSS2 KD animals. FIG. 13A depicts experimental data demonstrating that the genome-browser track views show the Cep152 gene locus (Chr2:125,603,000-125,626,000). FIG. 13B depicts experimental data demonstrating that the genome-browser track views show the Uimc gene locus (Chr5: 55,064,000-55,089,000). FIG. 13C depicts experimental data demonstrating that the genome-browser track views show the Nsmaf gene locus (Chr4: 6,425,000-6,464,000). The experiment was performed with 3 independent biological replicates per group.

FIG. 14, comprising FIG. 14A through FIG. 14F, depicts experimental data. FIG. 14A depicts Decile plots of genes enriched in H3K9ac show correlation with mRNA expression levels in hippocampus, in WT animals 1 hour following injection with EtOH. FIG. 14B depicts Decile plots of genes enriched in H3K27ac show correlation with mRNA expression levels in hippocampus, in WT animals 1 hour following injection with EtOH. FIG. 14C depicts in ACSS2 KD animals, the correlation between histone H3K9 acetylation and alcohol-related mRNA expression is largely lost (box-and-whisker plots show median value with whiskers extending to 1.5× the interquartile range; n=16,553 genes (population) arranged into ten equal-sized deciles by acetylation ChIP-seq enrichment). FIG. 14D depicts in ACSS2 KD animals, the correlation between histone H3K27 acetylation and alcohol-related mRNA expression is largely lost (box-and-whisker plots show median value with whiskers extending to 1.5× the interquartile range; n=16,553 genes (population) arranged into ten equal-sized deciles by acetylation ChIP-seq enrichment). FIG. 14E depicts GO analysis on H3K9ac peaks that are induced by EtOH in WT but not ACSS2 KD animals (n=332; Gene Ontology enrichment analysis performed using a modified Fisher's exact test (EASE) with the FDR controlled by the Yekutieli procedure, −log 10 of nominal P values are shown). FIG. 14F depicts GO analysis on H3K27ac peaks that are induced by EtOH in WT but not ACSS2 KD animals (n=480; Gene Ontology enrichment analysis performed using a modified Fisher's exact test (EASE) with the FDR controlled by the Yekutieli procedure, −log 10 of nominal P values are shown).

FIG. 15, comprising FIG. 15A through FIG. 15F, depicts experimental results. FIG. 15A depicts ACSS2i structure (C20H18N4O2S2; compound ADG-205). FIG. 15B depicts RNAseq showing differentially regulated genes in primary hippocampal neurons treated with 5 mM acetate (n=4 replicates per group; volcano plot of likelihood ratio test employed by DESeq2 (two-sided), FDR controlled for multiple hypothesis testing). FIG. 15C depicts gene ontology (GO) analysis of significantly upregulated (n=3613 genes) genes (GO analysis performed with GOrilla, using a minimal hypergeometric test). FIG. 15D depicts GO analysis of significantly downregulated (n=3987 genes) genes (GO analysis performed with GOrilla, using a minimal hypergeometric test). FIG. 15E depicts RNA-seq in primary hippocampal neurons isolated from C57/B16 mouse embryos and treated with acetate (5 mM) in the presence or absence of a small molecular inhibitor of ACSS2 (ACSS2i). 2107 of the 3613 acetate-induced genes fail to be upregulated in the presence of ACSS2i (box-and-whisker plots show median value with whiskers extending to 1.5× the interquartile range; n=3,613 induced genes (population) or 3,613 randomly sampled genes (population) tested using two-sided Mann-Whitney rank-sum test, P<2.2E-16)). FIG. 16F depicts a diagram. Shown in blue are acetate-induced genes in primary hippocampal neurons, together with the GO term analysis of ACSS2i sensitive genes (non-overlapping with yellow, which represents the genes that are upregulated by acetate in the presence of ACSS2i; n=2107, Gene Ontology enrichment analysis performed using a modified Fisher's exact test (EASE) with the FDR controlled by the Yekutieli procedure, −log 10 of nominal P values are shown).

FIG. 16, comprising FIG. 16A through FIG. 16F, depicts experimental results demonstrating ACSS2 mediated acetate-induced transcription in primary hippocampal neurons. FIG. 16A depicts RNA-seq in primary hippocampal neurons isolated from C57/B16 mouse embryos and treated with acetate (5 mM) in the presence or absence of a small molecular inhibitor of ACSS2 (ACSS2i). Heatmap showing 7,600 genes differentially expressed upon acetate treatment, and a third column showing the behavior of those genes under in the presence of the ACSS2 inhibitor. 2107 of the 3613 acetate-induced genes fail to be upregulated in the presence of ACSS2i (n=4 per group). FIG. 16B depicts GO term analysis of genes that are both sensitive to acetate and directly bound by ACSS2 (from ACSS2 ChIP-seq; n=429 genes, population assessment using modified Fisher's exact test (EASE) with the FDR corrected by the Yekutieli procedure, −log 10 of nominal P values are shown). FIG. 16C depicts HOMER unsupervised de novo motif analysis of ACSS2 hippocampal binding sites targeting acetate-sensitive genes (de novo motif analysis of 751 ACSS2 peaks, hypergeometric test for each motif comparing background set of ACSS2 peaks that do not target acetate sensitive genes). FIG. 16D depicts the overlap of genes upregulated by EtOH in vivo (dHPC) and acetate in vitro (n=830; hypergeometric test of gene set overlap, P=3.48e-237). FIG. 16E depicts ACSS2 target genes with alcohol-induced H3K9ac in vivo are upregulated by acetate in HPC neurons in vitro. ACSS2i blocks this gene induction (box-and-whisker plots show median value with whiskers extending to 1.5× the interquartile range; n=285 genes tested against an equal number of control genes using two-sided Mann-Whitney rank-sum test; P=0.0077). FIG. 16F depicts ACSS2 target genes with alcohol-induced H3K27ac in vivo are upregulated by acetate in HPC neurons in vitro. ACSS2i blocks this gene induction (box-and-whisker plots show median value with whiskers extending to 1.5× the interquartile range; n=362 genes tested against an equal number of control genes using two-sided Mann-Whitney rank-sum test; P=0.0013).

FIG. 17, comprising FIG. 17A through FIG. 17D, depicts genome-browser track views showing examples of gene up-regulation upon acetate treatment in hippocampal neurons, and diminished induction with ACSS2i treatment (n=4 per cohort). FIG. 17A depicts RNA-seq track views showing the Slc17a7 gene locus (Chr7: 45,162,500-45,179,000). FIG. 17B depicts RNA-seq track views showing the Ccnil gene locus (Chr11: 43,525,000-43,595,000). FIG. 17C depicts RNA-seq track views showing the Cpne7 gene locus (Chr8: 123,152,500-123,137,500). FIG. 17D depicts RNA-seq track views showing the Ndufv3 gene locus (Chr17: 31,523,000-31,534,000).

FIG. 18, comprising FIG. 18A and FIG. 18B, depicts experimental results. FIG. 18A depicts the cumulative number of ACSS2 peaks near the transcription start site (TSS) of acetylated ACSS2i sensitive genes, indicating that the majority ACSS2 binding events occurs over or proximal to the gene promoter. FIG. 18B depicts GO analysis for the 830 overlapping genes between the in vivo RNA-seq and ex vivo hippocampal neuron RNAseq (n=830 genes (population), Gene Ontology enrichment analysis performed using a modified Fisher's exact test (EASE) with the FDR controlled by the Yekutieli procedure).

FIG. 19, comprising FIG. 19A through FIG. 19E, depicts experimental results demonstrating ACSS2 is required for alcohol-induced associative learning. FIG. 19A depicts a schematic of ethanol-induced conditioned place preference (CPP). FIG. 19B depicts preference scores for the ethanol-paired chamber in wild-type (WT) mice (n=8; data are mean±s.e.m., Wilcoxon matched-pairs signed rank test, P=0.0391) and for the ethanol-paired chamber in mice with dorsal hippocampal knock-down (KD) of ACSS2 (n=10; data are mean±s.e.m., Wilcoxon matched-pairs signed rank test, P=0.4316). FIG. 19 C depicts a model. Acetate from hepatic alcohol breakdown is activated by neuronal ACSS2 in the brain and readily induces gene-regulatory histone acetylation. FIG. 19D depicts metabolized heavy d6-EtOH is incorporated into histone acetylation in the maternal brain. FIG. 19E depicts heavy label incorporation into histone acetylation in the fetal brain. Data represent the second of two pools of embryos (n=4 per pool) from maternal d6-EtOH injection. The Arachne plot axes represent the percentage of the third isotope of the acetylated peptide, corresponding to the D3 labeled form.

FIG. 20, comprising FIG. 20A through FIG. 20D, depicts experimental results. FIG. 20A depicts representative image showing virus localization to the dorsal hippocampus (dHPC) and Western blot (n=4 animals) showing dHPC ACSS2 levels in WT and ACSS2 KD mice (a.u.—arbitrary units; for gel source data, see Supplementary FIG. 1. FIG. 20B depicts quantification of ACSS2 protein levels in the dHPC and cortex of WT and dHPC ACSS2 KD mice (n=4 animals; data are mean±s.e.m., multiple T test, dHPC ACSS2 KD vs. WT, P=0.0001, q value=0.0001; Cortex ACSS2 KD vs. WT, P=0.2666, q value=0.1347). FIG. 20C depicts ACSS2 is required for alcohol-induced associative learning. Mean time (seconds/minute) spent in unconditioned and ethanol-conditioned chambers following ethanol-induced conditioned place preference training in WT (n=8) and dorsal hippocampal ACSS2 knock-down mice (n=10). Bar graphs represent mean+s.e.m. and show data points corresponding to individual animals. FIG. 20D depicts heavy label incorporation into histone acetylation in the fetal brain. Data represent the second of two pools of embryos (n=4 per pool) from maternal d6-EtOH injection. The Arachne plot axes represent the percentage of the third isotope of the acetylated peptide, corresponding to the D3 labeled form.

FIG. 21 depict experimental results demonstrating movement of mice in each cohort during day 1 of acquisition protocol, during habituation phase.

FIG. 22 depicts experimental results demonstrating levels of freezing of mice for each cohort during the acquisition protocol.

FIG. 23 depicts experimental results demonstrating levels of freezing of mice for each cohort during the contextual response and cued response analysis after acquisition.

FIG. 24 depicts experimental results demonstrating levels of freezing of mice throughout the cue presentation after acquisition phase, showing statistically significant reduction in the drug cohort EPV-018 (or ADG-205).

FIG. 25 depicts a schematic showing protocol for fear reconsolidation behavioral study in mice.

FIG. 26, comprising FIG. 26A through FIG. 26C, depicts experimental results of fear reconsolidation behavioral study in mice. FIG. 26A depicts a schematic showing protocol for fear reconsolidation behavioral study representing the fear acquisition protocol (day 0 in FIG. 25). FIG. 26B depicts a schematic showing protocol for fear reconsolidation behavioral study representing each of the reconsolidation sessions (days 1-5 and 8, with dosing done at days 1-4, 5 min before and 30 min after reconsolidation session). FIG. 26C depicts results of the fear reconsolidation behavioral study after administering DMOS or EPV-018 (ADG-205). Fisher's LSD test yields p values for three of the time points (0.5, 1.5, and 2.5 min).

FIG. 27 depicts experimental results demonstrating freezing behavior of mice during the respective days during the fear reconsolidation behavioral study in mice.

FIG. 28 depicts experimental results demonstrating that dorsal hippocampal ACSS2 knockdown significantly reduced the expression of cocaine-mediated conditioned place preference. The graph shows the difference in chamber preference between ACSS2 knock-down mice and wild-type after conditioning to chamber containing cocaine.

FIG. 29 depicts a graph showing the time spent interacting with an individual object relative to the total time spent interacting with all objects. DMSO injected mice spent significantly more time interacting with the object moved to a novel location compared to the ADG-205c treated mice. The animals treated with ADG-205c have very little preference for one object.

### DETAILED DESCRIPTION

The present invention relates to compositions and methods for treating neurological and cognitive diseases and disorders. In some embodiments, the invention provides compositions and methods for treating memory-related diseases and disorders. In various embodiments, the compositions and methods of the invention are useful in treating anxiety diseases and disorders such as phobias, panic disorders, psychosocial stress (e.g. as seen in disaster, catastrophe or violence victims), obsessive-compulsive disorder, generalized anxiety disorder and post-traumatic stress disorder (PTSD). In some embodiments, the compositions and methods of the invention are useful for regulating long term memory storage or consolidation.

The present invention also relates to compositions and methods for treating addiction and/or disease or disorders related to addiction. In various embodiments, the compositions and methods of the invention are useful for preventing or treating acute alcohol induced memory deficit and chronic alcohol induced memory deficit.

In some embodiments, the methods of the present invention comprise modulating chromatin acetylation. In one embodiment, the methods of the invention decrease chromatin acetylation. In one embodiment, the chromatin is neuronal chromatin. In one embodiment, the method comprises administering to a subject an effective amount of a composition comprising an inhibitor of ACSS2.

In certain instances, the compositions and methods described herein relate to inhibiting acetate-dependent acetyl-CoA synthetase 2 (ACSS2). In one embodiment, the composition of the present invention comprises an inhibitor of ACSS2. In one embodiment, the inhibitor of ACSS22 inhibits the expression, activity, or both, of ACSS2.

### Definitions

Unless defined otherwise, all technical and scientific terms used herein have the same meaning as commonly understood by one of ordinary skill in the art to which this invention belongs. Any methods and materials similar or equivalent to those described herein can be used in the practice or testing of the present invention, exemplary methods and materials are described.

Generally, the nomenclature used herein and the laboratory procedures in organic chemistry are those well-known and commonly employed in the art.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, or 0.1% from the specified value, as such variations are appropriate to perform the disclosed methods.

The term “abnormal” when used in the context of organisms, tissues, cells or components thereof, refers to those organisms, tissues, cells or components thereof that differ in at least one observable or detectable characteristic (e.g., age, treatment, time of day, etc.) from those organisms, tissues, cells or components thereof that display the “normal” (expected) respective characteristic. Characteristics which are normal or expected for one cell or tissue type, might be abnormal for a different cell or tissue type.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

A disease or disorder is “alleviated” if the severity of a sign or symptom of the disease or disorder, the frequency with which such a sign or symptom is experienced by a patient, or both, is reduced.

An “effective amount” or “therapeutically effective amount” of a compound is that amount of a compound which is sufficient to provide a beneficial effect to the subject to which the compound is administered.

The terms “patient,” “subject,” “individual,” and the like are used interchangeably herein, and refer to any animal, or cells thereof whether in vitro or in vivo, amenable to the methods described herein. In certain non-limiting embodiments, the patient, subject or individual is a human.

A “therapeutic” treatment is a treatment administered to a subject who exhibits signs or symptoms of a disease or disorder, for the purpose of diminishing or eliminating those signs or symptoms.

As used herein, “treating a disease or disorder” means reducing the severity and/or frequency with which a sign or symptom of the disease or disorder is experienced by a patient.

As used herein, the term “pharmaceutical composition” refers to a mixture of at least one compound useful within the invention with a pharmaceutically acceptable carrier. The pharmaceutical composition facilitates administration of the compound to a patient or subject. Multiple techniques of administering a compound exist in the art including, but not limited to, intravenous, oral, aerosol, parenteral, ophthalmic, pulmonary and topical administration.

As used herein, the term “pharmaceutically acceptable” refers to a material, such as a carrier or diluent, which does not abrogate the biological activity or properties of the compound, and is relatively non-toxic, i.e., the material may be administered to an individual without causing an undesirable biological effect or interacting in a deleterious manner with any of the components of the composition in which it is contained.

As used herein, the language “pharmaceutically acceptable salt” refers to a salt of the administered compound prepared from pharmaceutically acceptable non-toxic acids, including inorganic acids, organic acids, solvates, hydrates, or clathrates thereof. Examples of such inorganic acids are hydrochloric, hydrobromic, hydroiodic, nitric, sulfuric, phosphoric, acetic, hexafluorophosphoric, citric, gluconic, benzoic, propionic, butyric, sulfosalicylic, maleic, lauric, malic, fumaric, succinic, tartaric, amsonic, pamoic, p-tolunenesulfonic, and mesylic. Appropriate organic acids may be selected, for example, from aliphatic, aromatic, carboxylic and sulfonic classes of organic acids, examples of which are formic, acetic, propionic, succinic, camphorsulfonic, citric, fumaric, gluconic, isethionic, lactic, malic, mucic, tartaric, para-toluenesulfonic, glycolic, glucuronic, maleic, furoic, glutamic, benzoic, anthranilic, salicylic, phenylacetic, mandelic, embonic (pamoic), methanesulfonic, ethanesulfonic, pantothenic, benzenesulfonic (besylate), stearic, sulfanilic, alginic, galacturonic, and the like. Furthermore, pharmaceutically acceptable salts include, by way of non-limiting example, alkaline earth metal salts (e.g., calcium or magnesium), alkali metal salts (e.g., sodium-dependent or potassium), and ammonium salts.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the invention within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the invention, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound useful within the invention, and are physiologically acceptable to the patient. Supplementary active compounds may also be incorporated into the compositions. The “pharmaceutically acceptable carrier” may further include a pharmaceutically acceptable salt of the compound useful within the invention. Other additional ingredients that may be included in the pharmaceutical compositions used in the practice of the invention are known in the art and described, for example in Remington's Pharmaceutical Sciences (Genaro, Ed., Mack Publishing Co., 1985, Easton, Pa.), which is incorporated herein by reference.

As used herein, the term “potency” refers to the dose needed to produce half the maximal response (ED₅₀).

As used herein, the term “efficacy” refers to the maximal effect (Eₘₐₓ) achieved within an assay.

As used herein, the term “alkyl,” by itself or as part of another substituent means, unless otherwise stated, a straight or branched chain hydrocarbon having the number of carbon atoms designated (i.e. C₁₋₆ means one to six carbon atoms) and including straight, branched chain, or cyclic substituent groups. Examples include methyl, ethyl, propyl, isopropyl, butyl, isobutyl, tert-butyl, pentyl, neopentyl, hexyl, and cyclopropylmethyl.

As used herein, the term “substituted alkyl” means alkyl as defined above, substituted by one, two or three substituents selected from the group consisting of halogen, —OH, alkoxy, —NH₂, amino, azido, —N(CH₃)₂, —C(═O)OH, trifluoromethyl, —C≡N, —C(═O)O(C₁-C₄)alkyl, —C(═O)NH₂, —SO₂NH₂, —C(═NH)NH₂, and —NO₂. Examples of substituted alkyls include, but are not limited to, 2,2-difluoropropyl, 2-carboxycyclopentyl and 3-chloropropyl.

As used herein, the term “heteroalkyl” by itself or in combination with another term means, unless otherwise stated, a stable straight or branched chain alkyl group consisting of the stated number of carbon atoms and one or two heteroatoms selected from the group consisting of O, N, and S, and wherein the nitrogen and sulfur atoms may be optionally oxidized and the nitrogen heteroatom may be optionally quaternized. The heteroatom(s) may be placed at any position of the heteroalkyl group, including between the rest of the heteroalkyl group and the fragment to which it is attached, as well as attached to the most distal carbon atom in the heteroalkyl group. Examples include: —O—CH₂—CH₂—CH₃, —CH₂—CH₂—CH₂—OH, —CH₂—CH₂—NH—CH₃, —CH₂—S—CH₂—CH₃, and —CH₂CH₂—S(═O)—CH₃. Up to two heteroatoms may be consecutive, such as, for example, —CH₂—NH—OCH₃, or —CH₂—CH₂—S—S—CH₃

As used herein, the term “alkoxy” employed alone or in combination with other terms means, unless otherwise stated, an alkyl group having the designated number of carbon atoms, as defined above, connected to the rest of the molecule via an oxygen atom, such as, for example, methoxy, ethoxy, 1-propoxy, 2-propoxy (isopropoxy) and the higher homologs and isomers.

As used herein, the term “halo” or “halogen” alone or as part of another substituent means, unless otherwise stated, a fluorine, chlorine, bromine, or iodine atom.

As used herein, the term “cycloalkyl” refers to a mono cyclic or polycyclic non-aromatic radical, wherein each of the atoms forming the ring (i.e. skeletal atoms) is a carbon atom. In one embodiment, the cycloalkyl group is saturated or partially unsaturated. In another embodiment, the cycloalkyl group is fused with an aromatic ring. Cycloalkyl groups include groups having from 3 to 10 ring atoms. Illustrative examples of cycloalkyl groups include, but are not limited to, the following moieties:

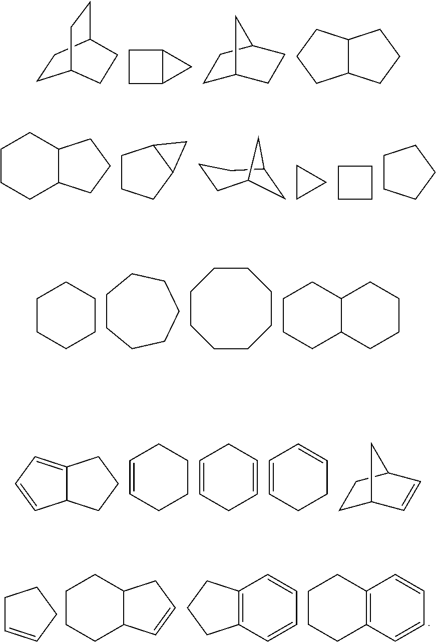

Monocyclic cycloalkyls include, but are not limited to, cyclopropyl, cyclobutyl, cyclopentyl, cyclohexyl, cycloheptyl, and cyclooctyl. Dicyclic cycloalkyls include, but are not limited to, tetrahydronaphthyl, indanyl, and tetrahydropentalene. Polycyclic cycloalkyls include adamantane and norbornane. The term cycloalkyl includes “unsaturated nonaromatic carbocyclyl” or “nonaromatic unsaturated carbocyclyl” groups, both of which refer to a nonaromatic carbocycle as defined herein, which contains at least one carbon double bond or one carbon triple bond.

As used herein, the term “heterocycloalkyl” or “heterocyclyl” refers to a heteroalicyclic group containing one to four ring heteroatoms each selected from O, S and N. In one embodiment, each heterocycloalkyl group has from 4 to 10 atoms in its ring system, with the proviso that the ring of said group does not contain two adjacent O or S atoms. In another embodiment, the heterocycloalkyl group is fused with an aromatic ring. In one embodiment, the nitrogen and sulfur heteroatoms may be optionally oxidized, and the nitrogen atom may be optionally quaternized. The heterocyclic system may be attached, unless otherwise stated, at any heteroatom or carbon atom that affords a stable structure. A heterocycle may be aromatic or non-aromatic in nature. In one embodiment, the heterocycle is a heteroaryl.

An example of a 3-membered heterocycloalkyl group includes, and is not limited to, aziridine. Examples of 4-membered heterocycloalkyl groups include, and are not limited to, azetidine and a beta lactam. Examples of 5-membered heterocycloalkyl groups include, and are not limited to, pyrrolidine, oxazolidine and thiazolidinedione. Examples of 6-membered heterocycloalkyl groups include, and are not limited to, piperidine, morpholine and piperazine. Other non-limiting examples of heterocycloalkyl groups are:

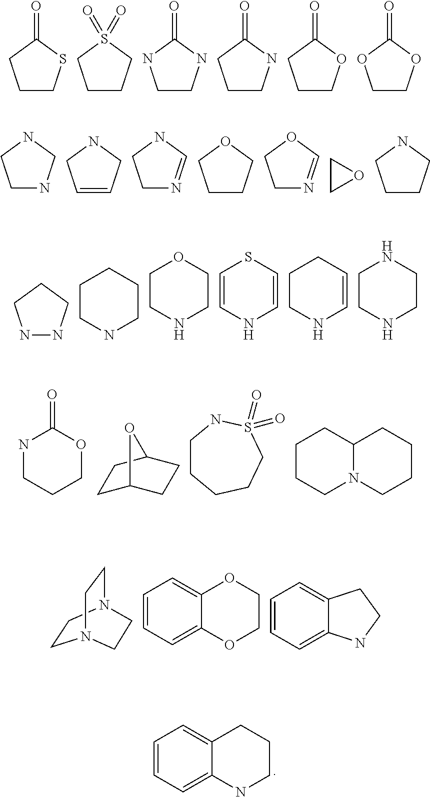

Examples of non-aromatic heterocycles include monocyclic groups such as aziridine, oxirane, thiirane, azetidine, oxetane, thietane, pyrrolidine, pyrroline, pyrazolidine, imidazoline, dioxolane, sulfolane, 2,3-dihydrofuran, 2,5-dihydrofuran, tetrahydrofuran, thiophane, piperidine, 1,2,3,6-tetrahydropyridine, 1,4-dihydropyridine, piperazine, morpholine, thiomorpholine, pyran, 2,3-dihydropyran, tetrahydropyran, 1,4-dioxane, 1,3-dioxane, homopiperazine, homopiperidine, 1,3-dioxepane, 4,7-dihydro-1,3-dioxepin, and hexamethyleneoxide.

As used herein, the term “aromatic” refers to a carbocycle or heterocycle with one or more polyunsaturated rings and having aromatic character, i.e. having (4n+2) delocalized π (pi) electrons, where n is an integer.

As used herein, the term “aryl,” employed alone or in combination with other terms, means, unless otherwise stated, a carbocyclic aromatic system containing one or more rings (typically one, two or three rings), wherein such rings may be attached together in a pendent manner, such as a biphenyl, or may be fused, such as naphthalene. Examples of aryl groups include phenyl, anthracyl, and naphthyl.

As used herein, the term “aryl-(C₁-C₃)alkyl” means a functional group wherein a one- to three-carbon alkylene chain is attached to an aryl group, e.g., —CH₂CH₂-phenyl. In one embodiment, aryl-(C₁-C₃)alkyl is aryl-CH₂- or aryl-CH(CH₃)—. The term “substituted aryl-(C₁-C₃)alkyl” means an aryl-(C₁-C₃)alkyl functional group in which the aryl group is substituted. Similarly, the term “heteroaryl-(C₁-C₃)alkyl” means a functional group wherein a one to three carbon alkylene chain is attached to a heteroaryl group, e.g., —CH₂CH₂-pyridyl. The term “substituted heteroaryl-(C₁-C₃)alkyl” means a heteroaryl-(C₁-C₃)alkyl functional group in which the heteroaryl group is substituted.

As used herein, the term “heteroaryl” or “heteroaromatic” refers to a heterocycle having aromatic character. A polycyclic heteroaryl may include one or more rings that are partially saturated. Examples include the following moieties:

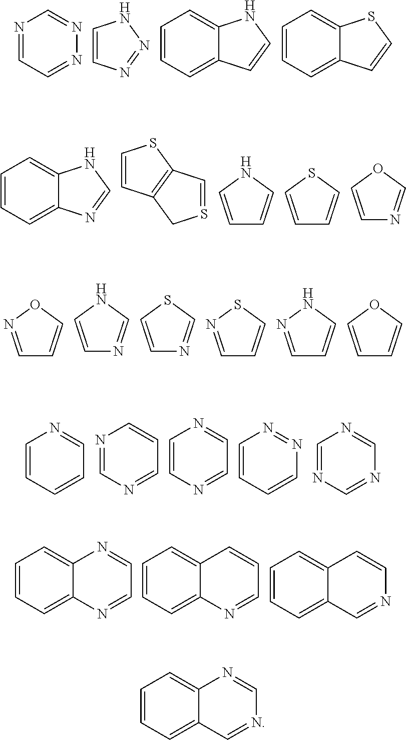

Examples of heteroaryl groups also include pyridyl, pyrazinyl, pyrimidinyl (particularly 2- and 4-pyrimidinyl), pyridazinyl, thienyl, furyl, pyrrolyl (particularly 2-pyrrolyl), imidazolyl, thiazolyl, oxazolyl, pyrazolyl (particularly 3- and 5-pyrazolyl), isothiazolyl, 1,2,3-triazolyl, 1,2,4-triazolyl, 1,3,4-triazolyl, tetrazolyl, 1,2,3-thiadiazolyl, 1,2,3-oxadiazolyl, 1,3,4-thiadiazolyl and 1,3,4-oxadiazolyl.

Examples of polycyclic heterocycles and heteroaryls include indolyl (particularly 3-, 4-, 5-, 6- and 7-indolyl), indolinyl, quinolyl, tetrahydroquinolyl, isoquinolyl (particularly 1- and 5-isoquinolyl), 1,2,3,4-tetrahydroisoquinolyl, cinnolinyl, quinoxalinyl (particularly 2- and 5-quinoxalinyl), quinazolinyl, phthalazinyl, 1,8-naphthyridinyl, 1,4-benzodioxanyl, coumarin, dihydrocoumarin, 1,5-naphthyridinyl, benzofuryl (particularly 3-, 4-, 5-, 6- and 7-benzofuryl), 2,3-dihydrobenzofuryl, 1,2-benzisoxazolyl, benzothienyl (particularly 3-, 4-, 5-, 6-, and 7-benzothienyl), benzoxazolyl, benzothiazolyl (particularly 2-benzothiazolyl and 5-benzothiazolyl), purinyl, benzimidazolyl (particularly 2-benzimidazolyl), benzotriazolyl, thioxanthinyl, carbazolyl, carbolinyl, acridinyl, pyrrolizidinyl, and quinolizidinyl.

As used herein, the term “substituted” means that an atom or group of atoms has replaced hydrogen as the substituent attached to another group. The term “substituted” further refers to any level of substitution, namely mono-, di-, tri-, tetra-, or penta-substitution, where such substitution is permitted. The substituents are independently selected, and substitution may be at any chemically accessible position. In one embodiment, the substituents vary in number between one and four. In another embodiment, the substituents vary in number between one and three. In yet another embodiment, the substituents vary in number between one and two.

As used herein, the term “optionally substituted” means that the referenced group may be substituted or unsubstituted. In one embodiment, the referenced group is optionally substituted with zero substituents, i.e., the referenced group is unsubstituted. In another embodiment, the referenced group is optionally substituted with one or more additional group(s) individually and independently selected from groups described herein.

In one embodiment, the substituents are independently selected from the group consisting of oxo, halogen, —CN, —NH₂, —OH, —NH(CH₃), —N(CH₃)₂, alkyl (including straight chain, branched and/or unsaturated alkyl), substituted or unsubstituted cycloalkyl, substituted or unsubstituted heterocycloalkyl, fluoro alkyl, substituted or unsubstituted heteroalkyl, substituted or unsubstituted alkoxy, fluoroalkoxy, —S-alkyl, S(═O)₂alkyl, —C(═O)NH[substituted or unsubstituted alkyl, or substituted or unsubstituted phenyl], —C(═O)N[H or alkyl]₂, —OC(═O)N[substituted or unsubstituted alkyl]₂, —NHC(═O)NH[substituted or unsubstituted alkyl, or substituted or unsubstituted phenyl], —NHC(═O)alkyl, —N[substituted or unsubstituted alkyl]C(═O)[substituted or unsubstituted alkyl], —NHC(═O)[substituted or unsubstituted alkyl], —C(OH)[substituted or unsubstituted alkyl]₂, and —C(NH₂)[substituted or unsubstituted alkyl]₂. In another embodiment, by way of example, an optional substituent is selected from oxo, fluorine, chlorine, bromine, iodine, —CN, —NH₂, —OH, —NH(CH₃), —N(CH₃)₂, —CH₃, —CH₂CH₃, —CH(CH₃)₂, —CF₃, —CH₂CF₃, —OCH₃, —OCH₂CH₃, —OCH(CH₃)₂, —OCF₃, —OCH₂CF₃, —S(═O)₂—CH₃, —C(═O)NH₂, —C(═O)—NHCH₃, —NHC(═O)NHCH₃, —C(═O)CH₃, —ON(O)₂, and —C(═O)OH. In yet one embodiment, the substituents are independently selected from the group consisting of C₁₋₆ alkyl, —OH, C₁₋₆ alkoxy, halo, amino, acetamido, oxo and nitro. In yet another embodiment, the substituents are independently selected from the group consisting of C₁₋₆ alkyl, C₁₋₆ alkoxy, halo, acetamido, and nitro. As used herein, where a substituent is an alkyl or alkoxy group, the carbon chain may be branched, straight or cyclic.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

The present invention relates to compositions and methods for treating or preventing a memory-related disease or disorder, such as, but not limited to, PTSD, addiction and addiction-related diseases or disorders. The present invention is based, in part, upon the finding that ACSS2 regulates histone acetylation and neuronal gene transcription. The inhibition of ACSS2 expression (such as by RNA interference) or ACSS2 activity (such as by a small molecule) decreases histone acetylation and impairs long-term spatial memory. Thus, the present invention relates to compositions and method to inhibit ACSS2 in order to inhibit histone acetylation and treat memory-related diseases or disorders.

In some embodiments, the composition of the present invention comprises an inhibitor of ACSS2 activity. In some embodiments, the composition comprises an inhibitor of ACSS2 expression. As demonstrated herein, compounds of the invention are useful for inhibiting ACCS2 activity. Compounds of the invention have also been found to be useful for inhibiting ACSS2 expression. Thus, in various embodiments, the composition comprises a compound of the invention that reduces the activity of ACSS2.

In some embodiments, the present invention provides a method for treating a neurological or cognitive disease or disorder in a subject. In one embodiment, the neurological or cognitive disease or disorder is a memory-related disease or disorder. In one embodiment, the method comprises administering to a subject an effective amount of a composition comprising a compound of the invention. In one embodiment, the method is useful in treating PTSD.

In another embodiment, the present invention provides a method for treating addiction or an addiction related disease or disorder in a subject. In some embodiments, the methods of the invention are useful for treating acute alcohol induced memory deficit. In other embodiments, the methods of the invention are useful for treating chronic alcohol induced memory deficit. In some embodiments, the methods comprise administering to a subject an effective amount of a composition comprising a compound of the invention.

### Compounds of the Invention

In one aspect, the present invention includes a compound of Formula (1):

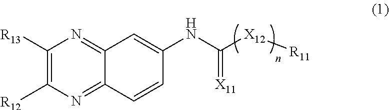

wherein, X₁₁ is selected from the group consisting of C(R₁₄)(R₁₅), O, S and NR₁₅; each occurrence of X₁₂ is selected from the group consisting of C(R₁₄)(R₁₅), O, S and NR₁₅;

R₁₁ is selected from the group consisting of —C₁-C₂₅ alkyl, cycloalkyl, heterocyclyl, aryl, heteroaryl, and combinations thereof, wherein R₁₁ are optionally substituted;

R₁₂ and R₁₃ are each independently selected from the group consisting of hydrogen, —C₁-C₆ alkyl, —C₃-C₆ aryl, and —C₄-C₆ heteroaryl, wherein R₁₂ and R₁₁ are optionally substituted;

each occurrence of R₁₄ and R₁₅ is independently selected from the group consisting of hydrogen, halogen, —OH, and C₁-C₆ alkyl; and

n is an integer from 0-4.

In one embodiment, in formula (1), X₁₁ is selected from the group consisting of C(R₁₄)(R₁₅), O, S and NR₁₅; each occurrence of X₁₂ is selected from the group consisting of C(R₁₄)(R₁₅), S and NR₁₅; R₁₁ is selected from the group consisting of —C₁-C₂₅ alkyl, cycloalkyl, heterocyclyl, aryl, heteroaryl, and combinations thereof, wherein R₁₁ is optionally substituted; R₁₂ and R₁₁ are each independently selected from the group consisting of hydrogen, —C₁-C₆ alkyl, —C₃-C₆ aryl, and —C₄-C₆ heteroaryl, wherein R₁₂ and R₁₃ are optionally substituted; each occurrence of R₁₄ and R₁₅ is independently selected from the group consisting of hydrogen, halogen, —OH, and C₁-C₆ alkyl; and n is an integer from 0-4.

In one embodiment, n is 0. In one embodiment, n is 1. In one embodiment, n is 2. In one embodiment, n is 3.

In one embodiment, R₁₁ is OR₁₅. In one embodiment, R₁₅ is alkyl. In one embodiment, R₁₅ is methyl.

In one embodiment, R₁₁ is piperidinyl.

In one embodiment, R₁₁ is morpholinyl.

In one embodiment, R₁₁ is pyrrolidinyl.

In one embodiment, R₁₁ is furanyl.

In one embodiment, R₁₁ is adamantyl.

In one embodiment, R₁₁ is substituted with a hydroxyl group.

In one embodiment, R₁₂ is alkyl. In one embodiment, R₁₂ is methyl.

In one embodiment, R₁₂ is a C₅-C₆ heteroaryl. In one embodiment, R₁₂ is a C₃-C₅ heteroaryl. In one embodiment, R₁₂ is furan. In one embodiment, R₁₂ is thiophenyl. In one embodiment, R₁₂ is pyridinyl.

In one embodiment, R₁₃ is alkyl. In one embodiment, R₁₃ is methyl.

In one embodiment, R₁₃ is a C₅-C₆ heteroaryl. In one embodiment, R₁₃ is a C₃-C₅ heteroaryl. In one embodiment, R₁₃ is furan. In one embodiment, R₁₃ is thiophenyl. In one embodiment, R₁₃ is pyridinyl.

In one embodiment, R₁₂ and R₁₃ are the same.

In another aspect, the present invention includes a compound of Formula (2):

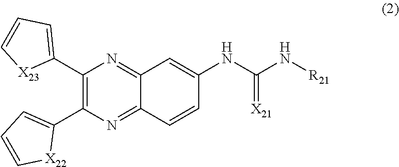

wherein, X₂₁ is O, or S;

X₂₂ and X₂₃ are each independently selected from the group consisting of NR₂₂, O, and S; and

R₂₁ is selected from the group consisting of —C₁-C₂₅ alkyl, cycloalkyl, heterocyclyl, aryl, heteroaryl, and combinations thereof, wherein R₁₁ is optionally substituted; and

each occurrence of R₂₂ is independently selected from the group consisting of hydrogen and C₁-C₆ alkyl.

In one embodiment, X₂₁ is O.

In one embodiment, X₂₂ is S.

In one embodiment, X₂₃ is S.

In one embodiment, R₂₁ is adamantyl.

In one embodiment, R₁₁ is cycloalkyl, which may be optionally substituted. In one embodiment, R₁₁ is —C₃-C₁₀ cycloalkyl, which may be optionally substituted. In one embodiment, R₂₁ is cycloalkyl, which may be optionally substituted. In one embodiment, R₂₁ is —C₃-C₁₀ cycloalkyl, which may be optionally substituted. In one embodiment, the cycloalkyl group is substituted. In one embodiment, the cycloalkyl group is unsubstituted. In one embodiment, the cycloalkyl group is monocyclic. Non-limiting examples of monocyclic cycloalkyl groups include cyclopropyl, cyclobutyl, cyclopentyl, cyclohexyl, cycloheptyl, cyclooctyl, cyclononyl, cyclodecyl, and the like. In another embodiment, the cycloalkyl group is polycyclic. For example, a polycyclic cycloalkyl group may be formed by joining two or more —C₃-C₁₀ cycloalkyl groups. Non-limiting examples of polycyclic cycloalkyl groups include adamantane and norbornane. In one embodiment, the cycloalkyl group is adamantyl, which may be optionally substituted. Cycloalkyl groups may also be dicyclic including, but not limited to, tetrahydronaphthyl, indanyl, and tetrahydropentalene. In one embodiment, the cycloalkyl group is saturated or partially unsaturated. Non-limiting examples of saturated or partially unsaturated cycloalkyl groups include cyclopentenyl, cyclopentadienyl, cyclohexenyl, cyclohexadienyl, cycloheptenyl, cycloheptadienyl, cycloheptatrienyl, cyclooctenyl, cycloocta-dienyl, cyclooctatrienyl, cyclooctatetraenyl, cyclononenyl, cyclononadienyl, cyclodecenyl, cyclodekadienyl, cyclooctynyl, cyclononynyl, cyclodecynyl, and the like. In one embodiment, the cycloalkyl group is fused with an aromatic ring.

In another aspect, the present invention includes a compound of Formula (3):

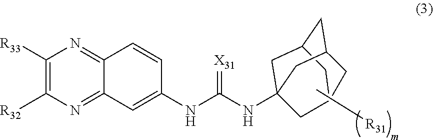

wherein, X₃₁ is selected from the group consisting of C(R₃₄)(R₃₅), O, S and NR₃₅;

each R₃₁ is independently hydrogen, —C₁-C₁₀ alkyl, halogen, —OH, or ═O or ═S formed by joining two R₃₁s,

R₃₂ and R₃₃ are each independently selected from the group consisting of hydrogen, —C₁-C₆ alkyl, —C₃-C₆ aryl, and —C₄-C₆ heteroaryl, wherein R₁₂ and R₁₃ are optionally substituted;

each occurrence of R₃₄ and R₃₅ is independently selected from the group consisting of hydrogen, halogen, —OH, and C₁-C₆ alkyl; and

m is an integer from 0-15.

In one embodiment, R₃₂ is alkyl. In one embodiment, R₃₂ is methyl.

In one embodiment, R₃₂ is a C₅-C₆ heteroaryl. In one embodiment, R₃₂ is a C₃-C₅ heteroaryl. In one embodiment, R₃₂ is furan. In one embodiment. R₃₂ is thiophenyl. In one embodiment, R₃₂ is pyridinyl.

In one embodiment, R₃₃ is alkyl. In one embodiment, R₃₃ is methyl.

In one embodiment, R₃₃ is a C₅-C₆ heteroaryl. In one embodiment, R₃₃ is a C₃-C₅ heteroaryl. In one embodiment, R₃₃ is furan. In one embodiment, R₃₃ is thiophenyl. In one embodiment, R₃₃ is pyridinyl.

In one embodiment, R₃₂ and R₃₃ are the same.

In one embodiment, the compound includes, but is not limited to:

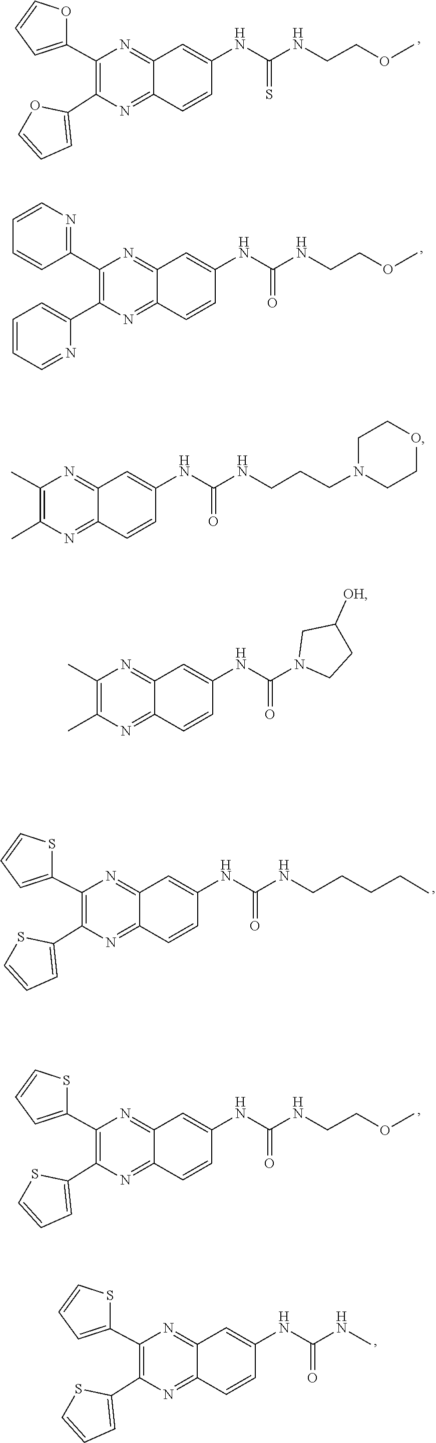

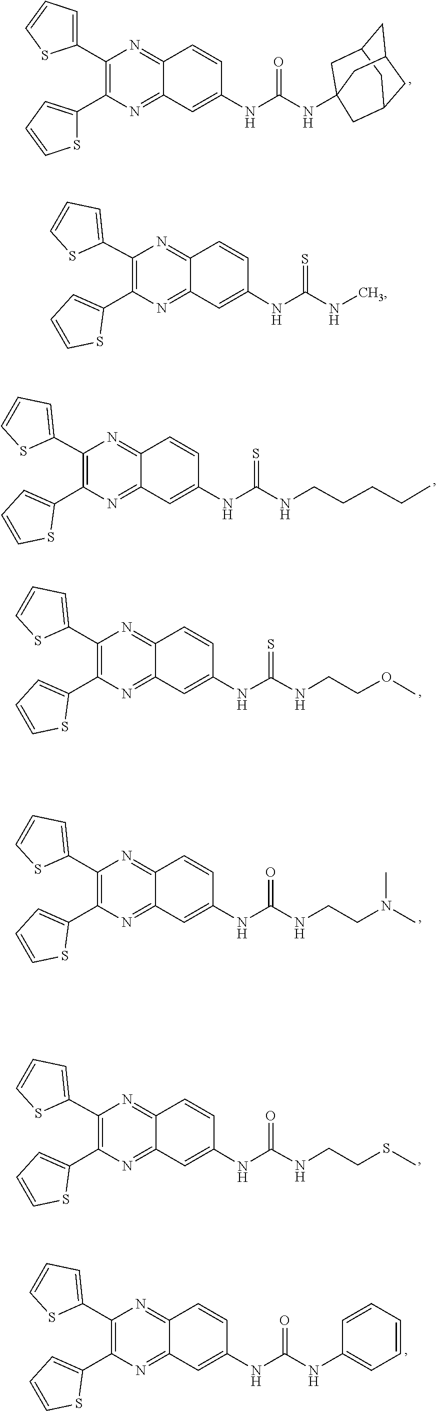

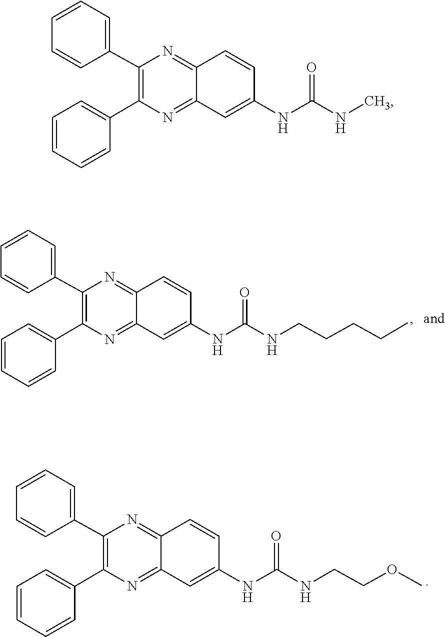 [SMILES: CNC(=O)Nc1ccc2nc(-c3ccccc3)c(-c3ccccc3)nc2c1 ; CCCCCNC(=O)Nc1ccc2nc(-c3ccccc3)c(-c3ccccc3)nc2c1 ; COCCNC(=O)Nc1ccc2nc(-c3ccccc3)c(-c3ccccc3)nc2c1]

In one embodiment, the compound is

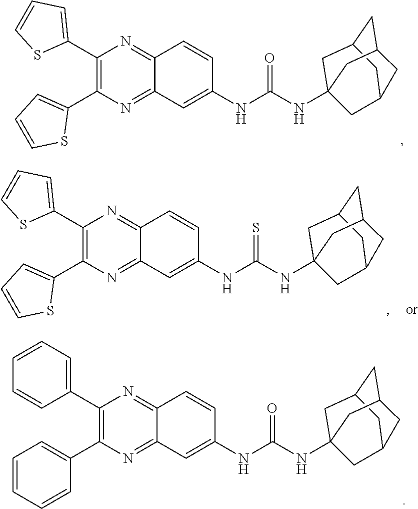

### Preparation of the Compounds of the Invention

Compounds of Formulae (1)-(3) may be prepared by the general schemes described herein, using the synthetic method known by those skilled in the art. The following examples illustrate non-limiting embodiments of the invention.

In a non-limiting embodiment, the synthesis of compounds of Formulae (1)-(3) is accomplished by treating 4-nitro-o-phenylenediamine (a) with a diketone (b) to form a 6-nitroquinoxaline (c), which is subsequently reduced via Pd/C-catalyzed hydrogenation to produce a 6-aminoquinoxaline (d). A diketone (a) can be produced using a method known in the art (Tet. Lett., 1995, 36:7305-7308, which is incorporated herein by reference in its entirety.)

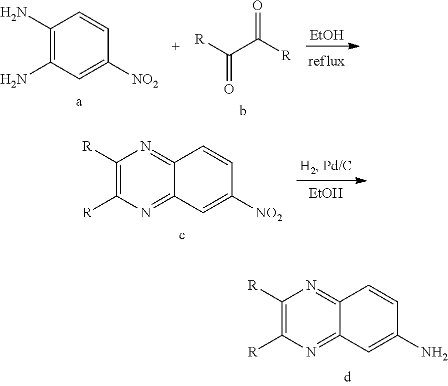

Quinoxaline d is then treated with an isocyanate to form a compound of Formulae (1)-(3).

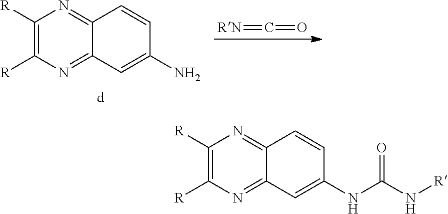

In another non-limiting embodiment, quinoxaline d is first treated with triphosgene, followed by the addition of an amine, to form a compound of Formulae (1)-(3).

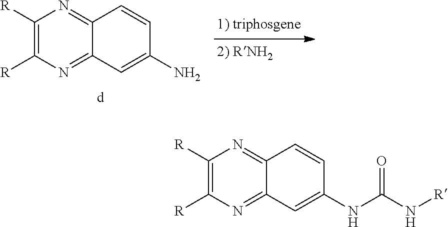

The compounds of the invention may possess one or more stereocenters, and each stereocenter may exist independently in either the R or S configuration. In one embodiment, compounds described herein are present in optically active or racemic forms. It is to be understood that the compounds described herein encompass racemic, optically-active, regioisomeric and stereoisomeric forms, or combinations thereof that possess the therapeutically useful properties described herein. Preparation of optically active forms is achieved in any suitable manner, including by way of non-limiting example, by resolution of the racemic form with recrystallization techniques, synthesis from optically-active starting materials, chiral synthesis, or chromatographic separation using a chiral stationary phase. In one embodiment, a mixture of one or more isomers is utilized as the therapeutic compound described herein. In another embodiment, compounds described herein contain one or more chiral centers. These compounds are prepared by any means, including stereoselective synthesis, enantioselective synthesis and/or separation of a mixture of enantiomers and/or diastereomers. Resolution of compounds and isomers thereof is achieved by any means including, by way of non-limiting example, chemical processes, enzymatic processes, fractional crystallization, distillation, and chromatography.

The methods and formulations described herein include the use of N-oxides (if appropriate), crystalline forms (also known as polymorphs), solvates, amorphous phases, and/or pharmaceutically acceptable salts of compounds having the structure of any compound of the invention, as well as metabolites and active metabolites of these compounds having the same type of activity. Solvates include water, ether (e.g., tetrahydrofuran, methyl tert-butyl ether) or alcohol (e.g., ethanol) solvates, acetates and the like. In one embodiment, the compounds described herein exist in solvated forms with pharmaceutically acceptable solvents such as water and ethanol. In another embodiment, the compounds described herein exist in unsolvated form.

In one embodiment, the compounds of the invention may exist as tautomers. All tautomers are included within the scope of the compounds presented herein.

Compounds described herein also include isotopically-labeled compounds wherein one or more atoms is replaced by an atom having the same atomic number, but an atomic mass or mass number different from the atomic mass or mass number usually found in nature. Examples of isotopes suitable for inclusion in the compounds described herein include and are not limited to ²H, ³H, ¹¹C, ¹³C, ¹⁴C, ³⁶Cl, ¹⁸F, ¹²³I, ¹²⁵I, ¹³N, ¹⁵N, ¹⁵O, ¹⁷O, ¹⁸O, ³²P, and ³⁵S. Isotopically-labeled compounds are prepared by any suitable method or by processes using an appropriate isotopically-labeled reagent in place of the non-labeled reagent otherwise employed.

In one embodiment, the compounds described herein are labeled by other means, including, but not limited to, the use of chromophores or fluorescent moieties, bioluminescent labels, or chemiluminescent labels.

The compounds described herein, and other related compounds having different substituents are synthesized using techniques and materials described herein and as described, for example, in Fieser & Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), Larock's Comprehensive Organic Transformations (VCH Publishers Inc., 1989), March, Advanced Organic Chemistry 4^{th} Ed., (Wiley 1992); Carey & Sundberg, Advanced Organic Chemistry 4th Ed., Vols. A and B (Plenum 2000,2001), and Green & Wuts, Protective Groups in Organic Synthesis 3rd Ed., (Wiley 1999) (all of which are incorporated by reference for such disclosure). General methods for the preparation of compound as described herein are modified by the use of appropriate reagents and conditions, for the introduction of the various moieties found in the formula as provided herein.

Compounds described herein are synthesized using any suitable procedures starting from compounds that are available from commercial sources, or are prepared using procedures described herein.

In one embodiment, reactive functional groups, such as hydroxyl, amino, imino, thio or carboxy groups, are protected in order to avoid their unwanted participation in reactions. Protecting groups are used to block some or all of the reactive moieties and prevent such groups from participating in chemical reactions until the protective group is removed. In another embodiment, each protective group is removable by a different means. Protective groups that are cleaved under totally disparate reaction conditions fulfill the requirement of differential removal.

In one embodiment, protective groups are removed by acid, base, reducing conditions (such as, for example, hydrogenolysis), and/or oxidative conditions. Groups such as trityl, dimethoxytrityl, acetal and t-butyldimethylsilyl are acid labile and are used to protect carboxy and hydroxy reactive moieties in the presence of amino groups protected with Cbz groups, which are removable by hydrogenolysis, and Fmoc groups, which are base labile. Carboxylic acid and hydroxy reactive moieties are blocked with base labile groups such as, but not limited to, methyl, ethyl, and acetyl, in the presence of amines that are blocked with acid labile groups, such as t-butyl carbamate, or with carbamates that are both acid and base stable but hydrolytically removable.

In one embodiment, carboxylic acid and hydroxy reactive moieties are blocked with hydrolytically removable protective groups such as the benzyl group, while amine groups capable of hydrogen bonding with acids are blocked with base labile groups such as Fmoc. Carboxylic acid reactive moieties are protected by conversion to simple ester compounds as exemplified herein, which include conversion to alkyl esters, or are blocked with oxidatively-removable protective groups such as 2,4-dimethoxybenzyl, while co-existing amino groups are blocked with fluoride labile silyl carbamates.

Allyl blocking groups are useful in the presence of acid- and base-protecting groups since the former are stable and are subsequently removed by metal or pi-acid catalysts. For example, an allyl-blocked carboxylic acid is deprotected with a palladium-catalyzed reaction in the presence of acid labile t-butyl carbamate or base-labile acetate amine protecting groups. Yet another form of protecting group is a resin to which a compound or intermediate is attached. As long as the residue is attached to the resin, that functional group is blocked and does not react. Once released from the resin, the functional group is available to react.

Typically blocking/protecting groups may be selected from:

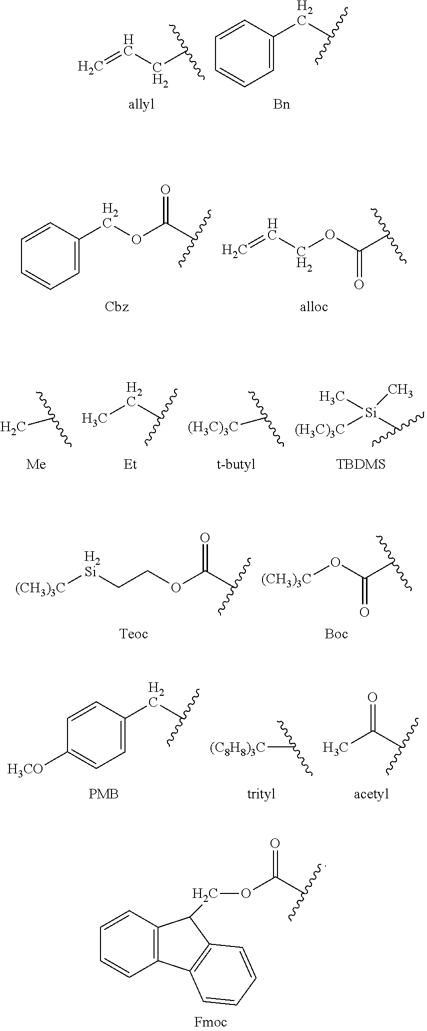

Other protecting groups, plus a detailed description of techniques applicable to the creation of protecting groups and their removal are described in Greene & Wuts, Protective Groups in Organic Synthesis, 3rd Ed., John Wiley & Sons, New York, N.Y., 1999, and Kocienski, Protective Groups, Thieme Verlag, New York, N.Y., 1994, which are incorporated herein by reference for such disclosure.

### Combinations

In some embodiments, the compositions of the present invention comprise a combination of compounds of the invention described herein. In certain embodiments, a composition comprising a combination of inhibitors described herein has an additive effect, wherein the overall effect of the combination is approximately equal to the sum of the effects of each individual inhibitor. In other embodiments, a composition comprising a combination of inhibitors described herein has a synergistic effect, wherein the overall effect of the combination is greater than the sum of the effects of each individual inhibitor.

In some embodiments, the composition of the present invention comprises a combination of a compound of the invention and a second therapeutic agent. For example, in one embodiment the second therapeutic agents include, but are not limited to, a PTSD treatment, an anxiety treatment, and a substance abuse treatment.

In some embodiments, the second therapeutic is a PTSD treatment. Exemplary therapeutics include, but are not limited to, anti-anxiety treatments, antidepressants, and adrenergic agents. In one embodiment, the PTSD treatment is a therapy treatment. For example, in one embodiment the PTSD treatment includes, psychotherapy, behavioral or cognitive behavioral therapy, eye movement desensitization and reprocessing (EMDR) group therapy, transcranial magnetic stimulation, deep brain stimulation and neurofeedback techniques, and medications including ketamine and d-cycloserine.

In one embodiment, administration of the compound of the invention in the emergency room or in intensive care units can be used for PTSD prophylaxis. In the peritraumatic phase, reactivated memory traces are vulnerable to disruption, thus administering a compound of the invention offers the potential to affect reconsolidation of trauma memories.

In some embodiments, the second therapeutic is a substance abuse treatment. For example, in one embodiment the substance abuse treatment includes, but is not limited to, naltrexone, disulfiram, acamprosate, topiramate, nicotine replacement therapy, nicotinic receptor antagonists, nicotinic receptor partial agonists, suboxone, levomethadyl acetate, dihydrocodeine, buprenorphine, ketamine, methadone, and dihydroetorphine.

A composition comprising a combination of compounds of the invention comprises individual compounds in any suitable ratio. For example, in one embodiment, the composition comprises a 1:1 ratio of two individual compounds. However, the combination is not limited to any particular ratio. Rather any ratio that is shown to be effective is encompassed.

### Methods

In some embodiments, the invention provides methods of inhibiting the ACSS2 in a subject in need thereof. In one embodiment, the method comprises administering to the subject an effective amount of a composition comprising an ACSS2 inhibitor.

In one embodiment, the invention provides a method for modulating chromatin acetylation in a subject. In one embodiment, the chromatin acetylation is histone acetylation. In one embodiment, the chromatin is neural chromatin. In one embodiment, methods of the invention modulate neuronal plasticity in a subject. In one embodiment, the method comprises administering to a subject an effective amount of a composition comprising an inhibitor of ACSS2. In one embodiment, the inhibitor of ACSS2 decreases histone acetylation.

In one aspect, the present invention provides a method for treating neurological or cognitive disease or disorder in a subject. In one embodiment, the neurological or cognitive disease or disorder is a memory-related disease or disorder. In one embodiment, the neurological or cognitive disease or disorder is a neuropsychiatric disorder. For example, in one embodiment the neuropsychiatric disorder includes, but is not limited to, anxiety disorders, psychotic disorders, mood disorders and somatoform disorders.

Exemplary neurological or cognitive diseases or disorders include, but are not limited to, post-traumatic stress disorder (PTSD), bipolar disorder, depression, Tourette's Syndrome, schizophrenia, obsessive-compulsive disorder, generalized anxiety disorder, panic disorders, phobias, personality disorders, including antisocial personality disorder, and other disorders involving troubling memories. In one embodiment, the neurological or cognitive diseases or disorders is PTSD.

In one embodiment, the method comprises (a) treating the subject with a compound of the invention during trauma recall and memory reconsolidation; and (b) subsequently treating the subject with cognitive behavioral therapy.

Exemplary cognitive behavioral therapy to be used in the method include, but are not limited to Cognitive Behavioral Therapies (CBT), Prolonged Exposure (PE), Cognitive Processing Therapy (CPT), and Eye Movement Desensitization and Reprocessing (EMDR). In one embodiment, the cognitive behavioral therapy is Cognitive Processing Therapy (CPT). Additional cognitive behavioral therapy are known in the art, for example in Yehuda et al., Post-Traumatic Stress Disorder, 2015, Nat Rev Dis Primers. 1: 15057, which is incorporated by reference in its entirety.

In one embodiment, the step of treating the subject with a compound of the invention during trauma recall and memory reconsolidation is repeated up to 12 times. In one embodiment, the step of treating the subject with a compound of the invention during trauma recall and memory reconsolidation is repeated at least 2 times, at least 3 times, at least 4 times, at least 5 times, at least 6 times, at least 7 times, at least 8 times, at least 9 times, at least 10 times, at least 11 times, or at least 12 times. In one embodiment, the step of treating the subject with a compound of the invention during trauma recall and memory reconsolidation is repeated 2, 3, 4, 5 or 6 times.

In another embodiment, the present invention provides a method for treating addiction or an addiction related disease or disorder in a subject. In one embodiment, the addiction includes, but is not limited to, addiction to: alcohol, tobacco, opioids, sedatives, hypnotics, anxiolytics, cocaine, cannabis, amphetamines, hallucinogens, inhalants, phencyclidine, impulse control disorders and behavioral addictions.

In one embodiment, the addiction is an alcohol addiction. In one embodiment, the method of the invention treats acute and/or chronic alcohol induced memory deficit.

In one embodiment, the invention provides a method for treating alcohol-related memory and cue-induced craving in augmented psychotherapy. In one embodiment, the method comprises administering to a subject an effective amount of a composition comprising an inhibitor of ACSS2. In one embodiment, the inhibitor of ACSS2 decreases histone acetylation. In one embodiment, the composition comprises a compound of the invention.

In one embodiment, the method comprises administering to the subject an effective amount of a composition that reduces or inhibits the expression or activity of ACSS2.

One of skill in the art will appreciate that the inhibitors of the invention can be administered singly or in any combination. Further, the inhibitors of the invention can be administered singly or in any combination in a temporal sense, in that they may be administered concurrently, or before, and/or after each other. One of ordinary skill in the art will appreciate, based on the disclosure provided herein, that the inhibitor compositions of the invention can be used to prevent or to treat an autoimmune disease or disorder, and that an inhibitor composition can be used alone or in any combination with another modulator to affect a therapeutic result. In various embodiments, any of the inhibitor compositions of the invention described herein can be administered alone or in combination with other modulators of other molecules associated with autoimmune diseases.

In one embodiment, the invention includes a method comprising administering a combination of inhibitors described herein. In certain embodiments, the method has an additive effect, wherein the overall effect of the administering a combination of inhibitors is approximately equal to the sum of the effects of administering each individual inhibitor. In other embodiments, the method has a synergistic effect, wherein the overall effect of administering a combination of inhibitors is greater than the sum of the effects of administering each individual inhibitor.

The method comprises administering a combination of inhibitors in any suitable ratio. For example, in one embodiment, the method comprises administering two individual inhibitors at a 1:1 ratio. However, the method is not limited to any particular ratio. Rather any ratio that is shown to be effective is encompassed.

### Pharmaceutical Compositions and Formulations

The invention also encompasses the use of pharmaceutical compositions of the invention or salts thereof to practice the methods of the invention. Such a pharmaceutical composition may consist of at least one modulator (e.g., inhibitor) composition of the invention or a salt thereof in a form suitable for administration to a subject, or the pharmaceutical composition may comprise at least one modulator (e.g., inhibitor) composition of the invention or a salt thereof, and one or more pharmaceutically acceptable carriers, one or more additional ingredients, or some combination of these. The compound of the invention may be present in the pharmaceutical composition in the form of a physiologically acceptable salt, such as in combination with a physiologically acceptable cation or anion, as is well known in the art.

In an embodiment, the pharmaceutical compositions useful for practicing the methods of the invention may be administered to deliver a dose of between 1 ng/kg/day and 100 mg/kg/day. In another embodiment, the pharmaceutical compositions useful for practicing the invention may be administered to deliver a dose of between 1 ng/kg/day and 500 mg/kg/day.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

Pharmaceutical compositions that are useful in the methods of the invention may be suitably developed for oral, rectal, vaginal, parenteral, topical, pulmonary, intranasal, buccal, ophthalmic, or another route of administration. A composition useful within the methods of the invention may be directly administered to the skin, or any other tissue of a mammal. Other contemplated formulations include liposomal preparations, resealed erythrocytes containing the active ingredient, and immunologically-based formulations. The route(s) of administration will be readily apparent to the skilled artisan and will depend upon any number of factors including the type and severity of the disease being treated, the type and age of the veterinary or human subject being treated, and the like.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

As used herein, a “unit dose” is a discrete amount of the pharmaceutical composition comprising a predetermined amount of the active ingredient. The amount of the active ingredient is generally equal to the dosage of the active ingredient that would be administered to a subject or a convenient fraction of such a dosage such as, for example, one-half or one-third of such a dosage. The unit dosage form may be for a single daily dose or one of multiple daily doses (e.g., about 1 to 4 or more times per day). When multiple daily doses are used, the unit dosage form may be the same or different for each dose.

In one embodiment, the compositions of the invention are formulated using one or more pharmaceutically acceptable excipients or carriers. In one embodiment, the pharmaceutical compositions of the invention comprise a therapeutically effective amount of a compound or conjugate of the invention and a pharmaceutically acceptable carrier. Pharmaceutically acceptable carriers that are useful, include, but are not limited to, glycerol, water, saline, ethanol and other pharmaceutically acceptable salt solutions such as phosphates and salts of organic acids. Examples of these and other pharmaceutically acceptable carriers are described in Remington's Pharmaceutical Sciences (1991, Mack Publication Co., New Jersey).

The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, isotonic agents, for example, sugars, sodium chloride, or polyalcohols such as mannitol and sorbitol, are included in the composition. Prolonged absorption of the injectable compositions may be brought about by including in the composition an agent that delays absorption, for example, aluminum monostearate or gelatin. In one embodiment, the pharmaceutically acceptable carrier is not DMSO alone.

Formulations may be employed in admixtures with conventional excipients, i.e., pharmaceutically acceptable organic or inorganic carrier substances suitable for oral, vaginal, parenteral, nasal, intravenous, subcutaneous, enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents, e.g., other analgesic agents.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” that may be included in the pharmaceutical compositions of the invention are known in the art and described, for example in Genaro, ed. (1985, Remington's Pharmaceutical Sciences, Mack Publishing Co., Easton, Pa.), which is incorporated herein by reference.

The composition of the invention may comprise a preservative from about 0.005% to 2.0% by total weight of the composition. The preservative is used to prevent spoilage in the case of exposure to contaminants in the environment. Examples of preservatives useful in accordance with the invention included but are not limited to those selected from the group consisting of benzyl alcohol, sorbic acid, parabens, imidurea and combinations thereof. An exemplary preservative is a combination of about 0.5% to 2.0% benzyl alcohol and 0.05% to 0.5% sorbic acid.

In one embodiment, the composition includes an anti-oxidant and a chelating agent that inhibits the degradation of the compound. Exemplary antioxidants for some compounds include BHT, BHA, alpha-tocopherol and ascorbic acid in the range of about 0.01% to 0.3%. In one embodiment, the antioxidant is BHT in the range of 0.03% to 0.1% by weight by total weight of the composition. In one embodiment, the chelating agent is present in an amount of from 0.01% to 0.5% by weight by total weight of the composition. Exemplary chelating agents include edetate salts (e.g. disodium edetate) and citric acid in the weight range of about 0.01% to 0.20%. In one embodiment, the chelating agent is in the range of 0.02% to 0.10% by weight by total weight of the composition. The chelating agent is useful for chelating metal ions in the composition that may be detrimental to the shelf life of the formulation. While BHT and disodium edetate are exemplary antioxidant and chelating agents, respectively, for some compounds, other suitable and equivalent antioxidants and chelating agents may be substituted therefore as would be known to those skilled in the art.

Liquid suspensions may be prepared using conventional methods to achieve suspension of the active ingredient in an aqueous or oily vehicle. Aqueous vehicles include, for example, water, and isotonic saline. Oily vehicles include, for example, almond oil, oily esters, ethyl alcohol, vegetable oils such as Arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, hydroxypropylmethylcellulose. Known dispersing or wetting agents include, but are not limited to, naturally-occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g., polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin, and acacia. Known preservatives include, but are not limited to, methyl, ethyl, or n-propyl-para-hydroxybenzoates, ascorbic acid, and sorbic acid. Known sweetening agents include, for example, glycerol, propylene glycol, sorbitol, sucrose, and saccharin. Known thickening agents for oily suspensions include, for example, beeswax, hard paraffin, and cetyl alcohol.

Liquid solutions of the active ingredient in aqueous or oily solvents may be prepared in substantially the same manner as liquid suspensions, the primary difference being that the active ingredient is dissolved, rather than suspended in the solvent. As used herein, an “oily” liquid is one which comprises a carbon-containing liquid molecule and which exhibits a less polar character than water. Liquid solutions of the pharmaceutical composition of the invention may comprise each of the components described with regard to liquid suspensions, it being understood that suspending agents will not necessarily aid dissolution of the active ingredient in the solvent. Aqueous solvents include, for example, water, and isotonic saline. Oily solvents include, for example, almond oil, oily esters, ethyl alcohol, vegetable oils such as Arachis, olive, sesame, or coconut oil, fractionated vegetable oils, and mineral oils such as liquid paraffin.

Powdered and granular formulations of a pharmaceutical preparation of the invention may be prepared using known methods. Such formulations may be administered directly to a subject, used, for example, to form tablets, to fill capsules, or to prepare an aqueous or oily suspension or solution by addition of an aqueous or oily vehicle thereto. Each of these formulations may further comprise one or more of dispersing or wetting agent, a suspending agent, and a preservative. Additional excipients, such as fillers and sweetening, flavoring, or coloring agents, may also be included in these formulations.

A pharmaceutical composition of the invention may also be prepared, packaged, or sold in the form of oil-in-water emulsion or a water-in-oil emulsion. The oily phase may be a vegetable oil such as olive or Arachis oil, a mineral oil such as liquid paraffin, or a combination of these. Such compositions may further comprise one or more emulsifying agents such as naturally occurring gums such as gum acacia or gum tragacanth, naturally-occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate. These emulsions may also contain additional ingredients including, for example, sweetening or flavoring agents.

Methods for impregnating or coating a material with a chemical composition are known in the art, and include, but are not limited to methods of depositing or binding a chemical composition onto a surface, methods of incorporating a chemical composition into the structure of a material during the synthesis of the material (i.e., such as with a physiologically degradable material), and methods of absorbing an aqueous or oily solution or suspension into an absorbent material, with or without subsequent drying.

The regimen of administration may affect what constitutes an effective amount. The therapeutic formulations may be administered to the subject either prior to or after a diagnosis of disease. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation. For example, in one embodiment, the in vivo efficacy at a single dose may vary based upon the pharmacokinetic and pharmacodynamic properties such as half-life. In one embodiment, the treatment regimen may be altered to adjust for these pharmacokinetic and pharmacodynamic properties. For example, in one embodiment, a compound with a shorter half-life can be dosed at more frequent intervals, at higher does, in different formulations or combinations thereof to achieve the same AUC.

Administration of the compositions of the present invention to a subject, for example, a mammal, including a human, may be carried out using known procedures, at dosages and for periods of time effective to prevent or treat disease. An effective amount of the therapeutic compound necessary to achieve a therapeutic effect may vary according to factors such as the activity of the particular compound employed; the time of administration; the rate of excretion of the compound; the duration of the treatment; other drugs, compounds or materials used in combination with the compound; the state of the disease or disorder, age, sex, weight, condition, general health and prior medical history of the subject being treated, and like factors well-known in the medical arts. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound of the invention is from about 1 and 5,000 mg/kg of body weight/per day. One of ordinary skill in the art would be able to study the relevant factors and make the determination regarding the effective amount of the therapeutic compound without undue experimentation.

The compound may be administered to a subject as frequently as several times daily, or it may be administered less frequently, such as once a day, once a week, once every two weeks, once a month, or even less frequently, such as once every several months or even once a year or less. It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on. The frequency of the dose will be readily apparent to the skilled artisan and will depend upon any number of factors, such as, but not limited to, the type and severity of the disease being treated, the type and age of the animal, etc.

Actual dosage levels of the active ingredients in the pharmaceutical compositions of this invention may be varied so as to obtain an amount of the active ingredient that is effective to achieve the desired therapeutic response for a particular subject, composition, and mode of administration, without being toxic to the subject.

A medical doctor, e.g., physician or veterinarian, having ordinary skill in the art may readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the subjects to be treated; each unit containing a predetermined quantity of therapeutic compound calculated to produce the desired therapeutic effect in association with the required pharmaceutical vehicle. The dosage unit forms of the invention are dictated by and directly dependent on (a) the unique characteristics of the therapeutic compound and the particular therapeutic effect to be achieved, and (b) the limitations inherent in the art of compounding/formulating such a therapeutic compound for the treatment of a disease in a subject.

In one embodiment, the compositions of the invention are administered to the subject in dosages that range from one to five times per day or more. In another embodiment, the compositions of the invention are administered to the subject in range of dosages that include, but are not limited to, once every day, every two, days, every three days to once a week, and once every two weeks. It will be readily apparent to one skilled in the art that the frequency of administration of the various combination compositions of the invention will vary from subject to subject depending on many factors including, but not limited to, age, disease or disorder to be treated, gender, overall health, and other factors. Thus, the invention should not be construed to be limited to any particular dosage regime and the precise dosage and composition to be administered to any subject will be determined by the attending physical taking all other factors about the subject into account.

Compounds of the invention for administration may be in the range of from about 1 mg to about 10,000 mg, about 20 mg to about 9,500 mg, about 40 mg to about 9,000 mg, about 75 mg to about 8,500 mg, about 150 mg to about 7,500 mg, about 200 mg to about 7,000 mg, about 3050 mg to about 6.000 mg, about 500 mg to about 5,000 mg, about 750 mg to about 4,000 mg, about 1 mg to about 3,000 mg, about 10 mg to about 2,500 mg, about 20 mg to about 2,000 mg, about 25 mg to about 1,500 mg, about 50 mg to about 1,000 mg, about 75 mg to about 900 mg, about 100 mg to about 800 mg, about 250 mg to about 750 mg, about 300 mg to about 600 mg, about 400 mg to about 500 mg, and any and all whole or partial increments there between.

In some embodiments, the dose of a compound of the invention is from about 1 mg and about 2,500 mg. In some embodiments, a dose of a compound of the invention used in compositions described herein is less than about 10,000 mg, or less than about 8,000 mg, or less than about 6,000 mg, or less than about 5,000 mg, or less than about 3,000 mg, or less than about 2,000 mg, or less than about 1,000 mg, or less than about 500 mg, or less than about 200 mg, or less than about 50 mg. Similarly, in some embodiments, a dose of a second compound (i.e., a drug used for treating the same or another disease as that treated by the compositions of the invention) as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

In one embodiment, the present invention is directed to a packaged pharmaceutical composition comprising a container holding a therapeutically effective amount of a compound of the invention, alone or in combination with a second pharmaceutical agent; and instructions for using the compound to treat, prevent, or reduce one or more symptoms of a disease in a subject.

The term “container” includes any receptacle for holding the pharmaceutical composition. For example, in one embodiment, the container is the packaging that contains the pharmaceutical composition. In other embodiments, the container is not the packaging that contains the pharmaceutical composition, i.e., the container is a receptacle, such as a box or vial that contains the packaged pharmaceutical composition or unpackaged pharmaceutical composition and the instructions for use of the pharmaceutical composition. Moreover, packaging techniques are well known in the art. It should be understood that the instructions for use of the pharmaceutical composition may be contained on the packaging containing the pharmaceutical composition, and as such the instructions form an increased functional relationship to the packaged product. However, it should be understood that the instructions may contain information pertaining to the compound's ability to perform its intended function, e.g., treating or preventing a disease in a subject, or delivering an imaging or diagnostic agent to a subject.

Routes of administration of any of the compositions of the invention include oral, nasal, parenteral, sublingual, transdermal, transmucosal (e.g., sublingual, lingual, (trans)buccal, and (intra)nasal), intravesical, intraduodenal, intragastrical, rectal, intraperitoneal, subcutaneous, intramuscular, intradermal, intra-arterial, intravenous, or administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present invention are not limited to the particular formulations and compositions that are described herein.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore are not to be construed as limiting in any way the remainder of the disclosure.

### Example 1: Synthesis of ADG-20

### Synthesis of di(thiophenyl)quinoxalinamine

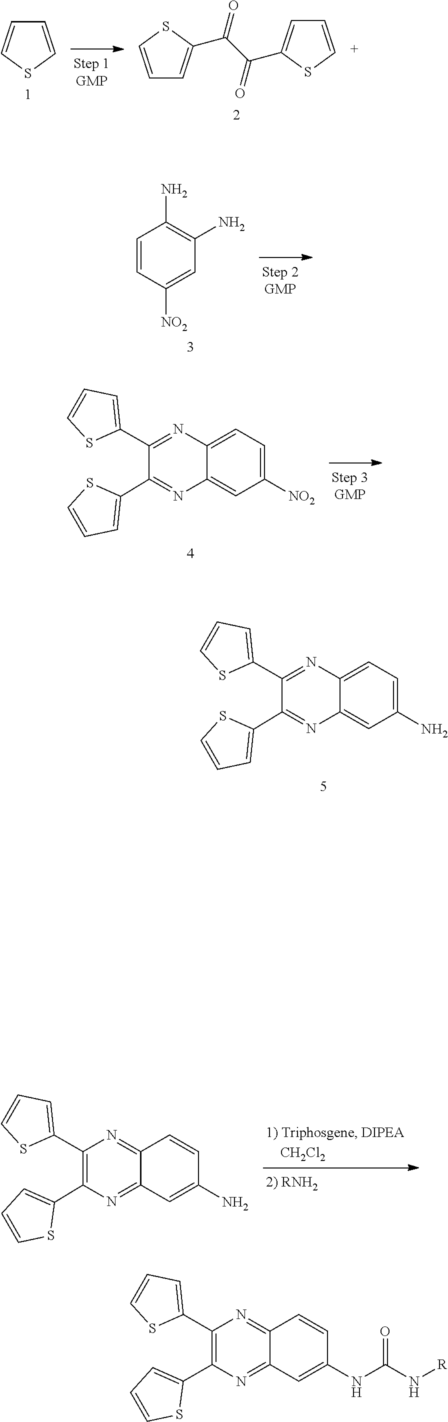

### ADG-I-204: 1-2,3-di(thiophen-2-yl)quinoxalin-6-yl)-3-pentylurea (R=n-C₅H₁₁)

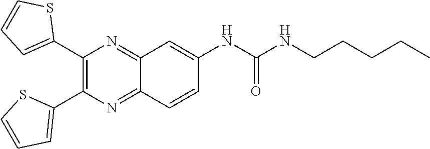 [SMILES: CCCCCNC(=O)Nc1ccc2nc(-c3cccs3)c(-c3cccs3)nc2c1]

To a stirring solution of 2,3-di(thiophen-2-yl)quinoxalin-6-amine (1.04 g, 3.36 mmol, 1 eq) in anhydrous CH₂Cl₂ (34 mL) was added N,N-diisopropylethylamine (1.17 mL, 6.72 mmol, 2 eq) followed by triphosgene (329 mg, 1.11 mmol, 0.33 eq) in anhydrous CH₂Cl₂ (1 mL, final concentration 0.1M) to give a red-orange solution. The reaction mixture was allowed to stir for 4 h at room temperature, then amylmine (0.49 mL, 4.20 mmol, 1.25 eq) was added dropwise. The reaction mixture was then allowed to stir for 16 h at room temperature. A stream of argon was blown over the reaction mixture to remove the solvent and any excess phosgene, and the residue obtained was purified by flash chromatography (50-60% EtOAc/Hexanes) to afford the title compound as a yellow solid (872 mg, 61%). ¹H NMR (500 MHz, DMSO-d₆) δ 9.07 (s, 1H), 8.23 (d, J=2.3 Hz, 1H), 7.90 (d, J=9.0 Hz, 1H), 7.76 (dd, J=9.7, 5.0 Hz, 2H), 7.69 (dd, J=9.1, 2.4 Hz, 1H), 7.16 (dd, J=16.6, 3.6 Hz, 2H), 7.13-7.05 (m, 2H), 6.41 (t, J=5.7 Hz, 1H), 3.14 (q, J=6.5 Hz, 2H), 1.48 (p, J=7.1 Hz, 2H), 1.40-1.11 (n, 4H, overlapping with grease), 0.90 (t, J=6.7 Hz, 3H). ¹³C NMR (126 MHz, DMSO) δ 154.87, 146.09, 143.13, 142.59, 141.40, 141.19, 141.08, 135.75, 129.67, 129.00, 128.91, 128.74, 128.66, 127.77, 127.65, 123.77, 111.49, 29.26, 28.55, 21.83, 13.91. HRMS (ESI) m/z calc'd for C₂₂H₂₃N₄OS₂ [M+H]⁺ 423.1313, found 423.1336.

### ADG-I-205: 1-(2,3-di(thiophen-2-yl)quinoxalin-6-yl)-3-(2-methoxyethyl)urea (R=MeOCH₂CH₂)

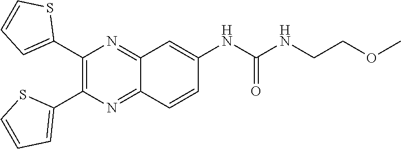 [SMILES: COCCNC(=O)Nc1ccc2nc(-c3cccs3)c(-c3cccs3)nc2c1]

To a stirring solution of 2,3-di(thiophen-2-yl)quinoxalin-6-amine (337 mg, 1.09 mmol, 1 eq) in anhydrous CH₂Cl₂ (5.5 mL) was added N,N-diisopropylethylamine (0.38 mL, 2.18 mmol, 2 eq) followed by triphosgene (107 mg, 0.36 mmol, 0.33 eq) in anhydrous CH₂Cl₂ (5.5 mL) to give a red-orange solution. The reaction mixture was allowed to stir for 4 h at room temperature, then 2-methoxyethylamine (0.12 mL, 1.36 mmol, 1.25 eq) was added dropwise. The reaction mixture was then allowed to stir for 16 h at room temperature. A stream of argon was blown over the reaction mixture to remove the solvent and any excess phosgene, and the residue obtained was purified by flash chromatography (70% EtOAc/Hexanes) to afford the title compound as a yellow solid (196 mg, 50%). ¹H NMR (500 MHz, DMSO-d₆) δ 9.19 (s, 1H), 8.23 (d, J=2.4 Hz, 1H), 7.91 (d, J=9.1 Hz, 1H), 7.76 (ddd, J=9.7, 5.1, 1.1 Hz, 2H), 7.68 (dd, J=9.1, 2.4 Hz, 1H), 7.18 (dd, J=3.7, 1.2 Hz, 1H), 7.15 (dd, J=3.7, 1.1 Hz, 1H), 7.10 (ddd, J=6.9, 5.1, 3.7 Hz, 2H), 6.47 (t, J=5.6 Hz, 1H), 3.43 (t, J=5.4 Hz, 2H), 3.33 (t, J=5.5 Hz, 2H), 3.30 (s, 3H). ¹³C NMR (126 MHz, DMSO) δ 154.83, 146.12, 143.21, 142.42, 141.37, 141.17, 141.07, 135.79, 129.68, 129.02, 128.94, 128.80, 128.68, 127.77, 127.65, 123.70, 111.55, 71.06, 57.90, 38.87. HRMS (ESI) m/z calc'd for C₂₀H₁₉N₄O₂S₂ [M+H]⁺ 411.0949, found 411.0926.

### ADG-I-206: 1-(2,3-di(thiophen-2-yl)quinoxalin-6-yl)-3-methylurea (R=Me)

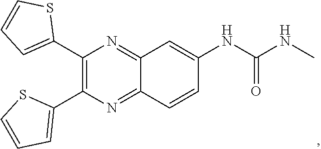 [SMILES: CNC(=O)Nc1ccc2nc(-c3cccs3)c(-c3cccs3)nc2c1]

To a stirring solution of 2,3-di(thiophen-2-yl)quinoxalin-6-amine (333 mg, 1.08 mmol, 1 eq) in anhydrous CH₂C₁₂ (5.4 mL) was added N,N-diisopropylethylamine (0.375 mL, 2.15 mmol, 2 eq) followed by triphosgene (105 mg, 0.36 mmol, 0.33 eq) in anhydrous CH₂Cl₂ (5.4 mL) to give a red-orange solution. The reaction mixture was allowed to stir for 4 h at room temperature, then methylamine (2M in THF, 0.67 mL, 1.35 mmol, 1.25 eq) was added dropwise. The reaction mixture was then allowed to stir for 16 h at room temperature. A stream of argon was blown over the reaction mixture to remove the solvent and any excess phosgene, and the residue obtained was purified by flash chromatography (80% EtOAc/Hexanes) to afford the title compound as a yellow solid (196 mg, 50%). ¹H NMR (500 MHz, DMSO-d₆) δ 9.17 (s, 1H), 8.24 (d, J=2.4 Hz, 1H), 7.90 (d, J=9.0 Hz, 1H), 7.84-7.54 (m, 3H), 7.16 (dd, J=13.9, 3.7 Hz, 2H), 7.12-7.06 (m, 2H), 6.29 (q, J=4.6 Hz, 1H), 2.71 (d, J=4.6 Hz, 3H). ¹³C NMR (126 MHz, DMSO) δ 155.51, 146.08, 143.15, 142.62, 141.39, 141.19, 141.10, 135.76, 129.65, 129.00, 128.91, 128.72, 128.66, 127.77, 127.65, 123.80, 111.56, 26.29. HRMS (ESI) m/z calc'd for C₁₈H₁₅N₄OS₂ [M+H]⁺367.0687, found 367.0689.

### ADG-207: 1-((1S,3s)-adamantan-1-yl)-3-(2,3-di(thiophen-2-yl)quinoxalin-6-yl)urea (R=1-adamantyl)

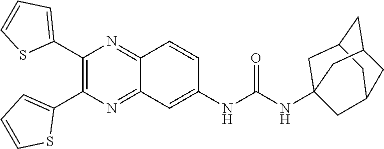 [SMILES: O=C(Nc1ccc2nc(-c3cccs3)c(-c3cccs3)nc2c1)NC12CC3CC(CC(C3)C1)C2]

To a stirring solution of 2,3-di(thiophen-2-yl)quinoxalin-6-amine (32 mg, 0.1 mmol, 1 eq) in anhydrous CH₂Cl₂ (0.6 mL) was added N,N-diisopropylethylamine (0.04 mL, 0.2 mmol, 2 eq) followed by triphosgene (10 mg, 0.034 mmol, 0.33 eq) in anhydrous CH₂Cl₂ (0.6 mL, final concentration 0.08 M) to give a red-orange solution. The reaction mixture was allowed to stir for 4 h at room temperature, then 1-adamantanamine (0.49 mL, 4.20 mmol, 1.25 eq) was added dropwise. The reaction mixture was then allowed to stir for 16 h at room temperature. A stream of argon was blown over the reaction mixture to remove the solvent and any excess phosgene, and the residue obtained was purified by flash chromatography (40% EtOAc/Hexanes) to afford the title compound contaminated with 1,1-di-adamantanylurea. The product was re-purified by flash chromatography twice to afford the analytically pure title compound as a yellow solid (4 mg, 8%) ¹H NMR (500 MHz, DMSO-d₆) δ 8.91 (s, 1H), 8.20 (d, J=2.4 Hz, 1H), 7.89 (d, J=9.1 Hz, 1H), 7.76 (ddd, J=9.2, 5.1, 1.2 Hz, 2H), 7.61 (dd, J=9.1, 2.4 Hz, 1H), 7.19 (dd, J=3.7, 1.2 Hz, 1H), 7.14 (dd, J=3.7, 1.2 Hz, 1H), 7.10 (ddd, J=11.0, 5.0, 3.6 Hz, 2H), 6.15 (s, 1H), 2.06 (s, 3H), 1.99 (d, J=2.9 Hz, 6H), 1.66 (t, J=3.1 Hz, 6H). ¹³C NMR (126 MHz, DMSO) δ 153.55, 146.09, 143.03, 142.57, 141.48, 141.25, 141.06, 135.67, 129.73, 128.98, 128.87, 128.76, 128.65, 127.77, 127.64, 123.66, 111.24, 50.15, 41.50, 36.00, 28.88. HRMS (ESI) m/z calc'd for C₂₇H₂₇N₄OS₂ [M+H]⁺ 487.1626, found 487.1625.

### Example 2: Small Molecule Inhibition of ACSS2

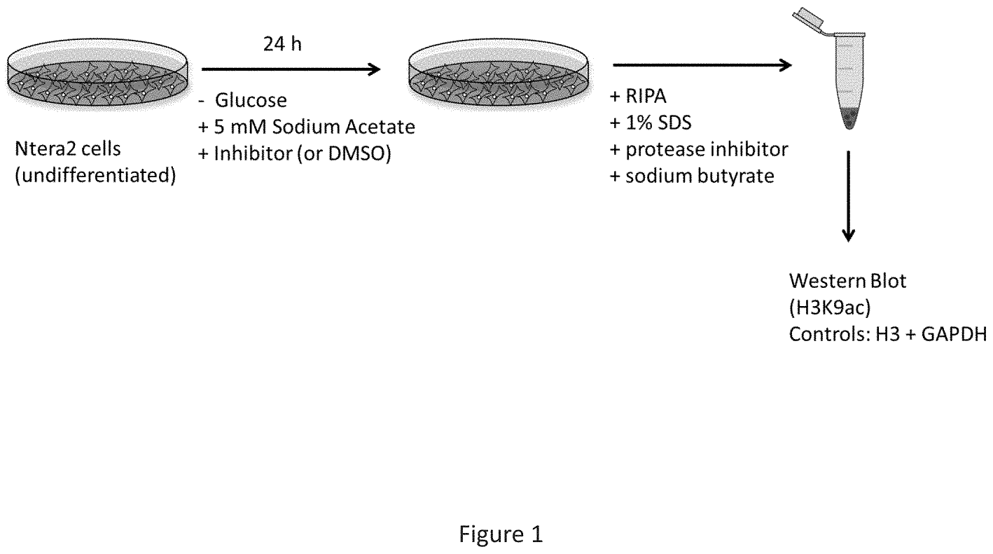

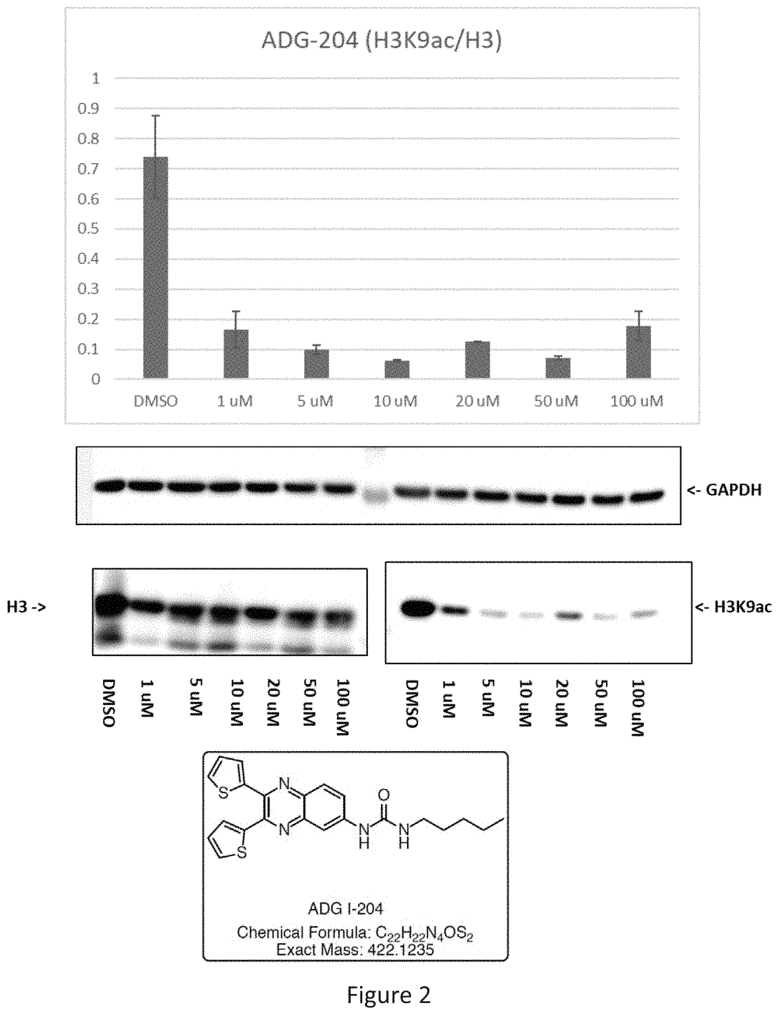

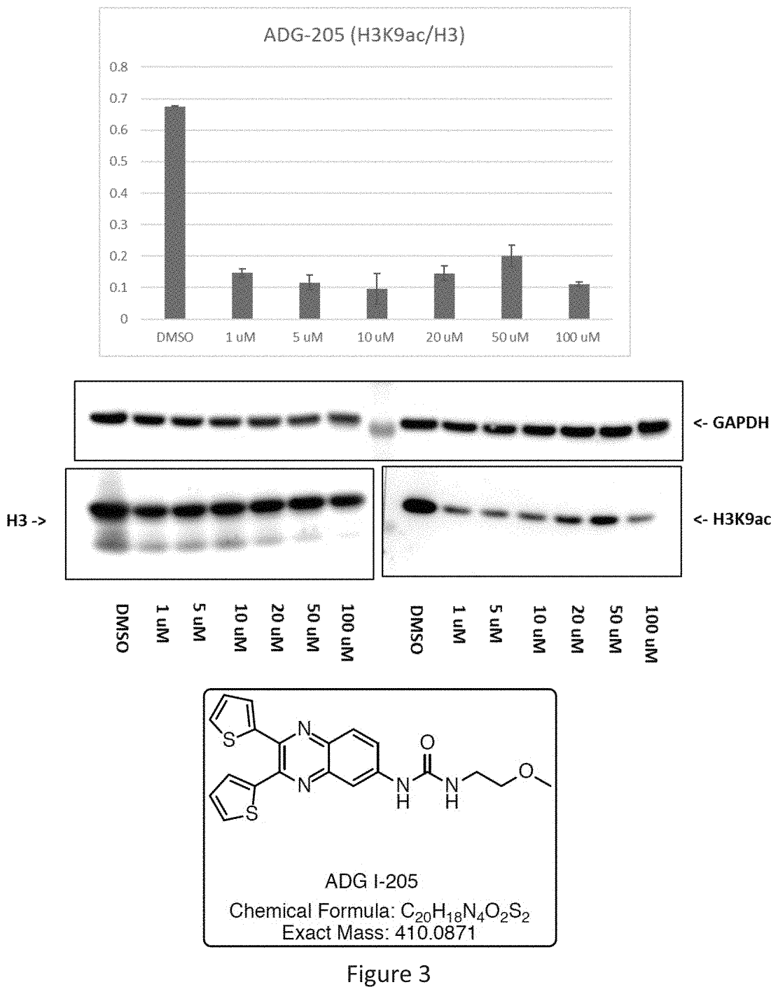

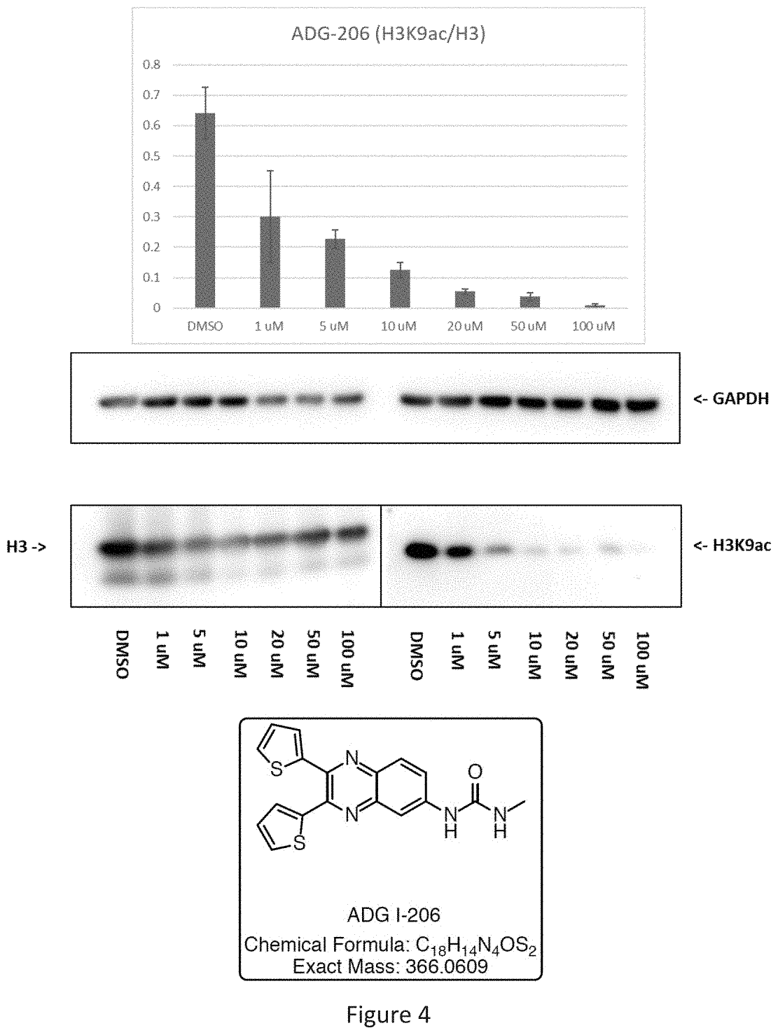

Undifferentiated Ntera2 cells were treated with inhibitor for 24 hours with ADG-204, ADG-205 or ADG-206 (FIG. 1). Western blots were used to determine the levels H3K3ac after treatment with ADG-204 (FIG. 2), ADG-205 (FIG. 3) or ADG-206 (FIG. 4).

### Example 3: Inhibition of ACSS2

To investigate the role of ACSS2 in the adult hippocampus, ACSS2 expression is attenuated in the dorsal hippocampus by treatment with small molecule ACSS2 inhibitors ADG-204, ADG-205, ADG-206 or ADG-207.

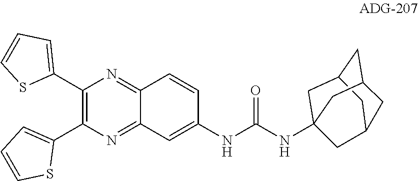 [SMILES: O=C(Nc1ccc2nc(-c3cccs3)c(-c3cccs3)nc2c1)NC12CC3CC(CC(C3)C1)C2]

Compared to control-treated mice, Mice treated with an ACSS2 inhibitor show similar levels of locomotion, coordination, body weight, and anxiety-related thigmotaxis during open field exploration; therefore, ACSS2 inhibition does not cause gross behavioral alterations.

To assess hippocampus-dependent spatial memory, an object-location memory paradigm is used. Animals explore three different objects during training, and long-term memory is tested by re-exposure 24 hours later with one object moved to a different location. In training, control and inhibitor treated mice show no difference in exploration. During memory retrieval, control mice show increased exploration of the object that had been moved. By contrast, mice treated with an ACSS2 inhibitor are impaired in spatial object memory and display a lower discrimination index. Mice treated with an ACSS2 show reduced total object exploration during the test, suggesting diminished novelty associated with intact recognition of the objects from the training session.

As a control experiment, control mice or mice treated with an ACSS2 inhibitor are subjected to a contextual fear conditioning paradigm. During the 24-hour test session, there are no significant difference in the amount of freezing behavior between control mice or mice treated with an ACSS2 inhibitor suggesting that the ventral hippocampus successfully mediates context-shock association. Overall, ACSS2 has a critical role in dorsal hippocampus-mediated long-term spatial memory.

### Example 4: Inhibition of Acetyl-CoA Synthetase Prevents the Incorporation of Alcohol-Derived Heavy Acetyl Groups into Histone Acetylation

To investigate the direct role of ACSS2 in alcohol-dependent acetylation in the brain, mice are treated with an ACSS2 inhibitor, ADG-204, ADG-205, ADG-206, or ADG-207. Treatment with an ACSS2 inhibitor prevents the incorporation of alcohol-derived heavy acetyl groups into histone acetylation. In contrast, in control mice, vHPC incorporation of the heavy label is not affected. Thus, acetate derived from hepatic alcohol metabolism is transported to the brain and readily incorporated into histone acetylation.

### Example 5: Pharmacokinetics of ACSS2 Inhibitors

The data presented herein demonstrates the pharmacokinetics of ACSS2 inhibitors ADG I-204, ADG I-205, ADG I-206, and ADG I-207.

Table 1 depicts the compound structures and properties.

TABLE 1

| Compound | Structure | Name |
|---|---|---|
| ADG 1-204 | 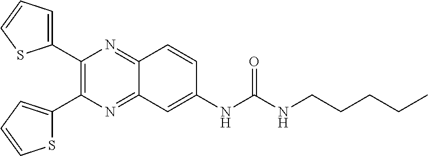 [SMILES: CCCCCNC(=O)Nc1ccc2nc(-c3cccs3)c(-c3cccs3)nc2c1] | 1-(2,3-di(thiophen-2- yl)quinoxalin-6-yl)-3- pentylurea |
| ADG 1-205 | 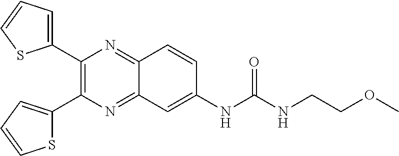 [SMILES: COCCNC(=O)Nc1ccc2nc(-c3cccs3)c(-c3cccs3)nc2c1] | 1-(2,3-di(thiophen-2- yl)quinoxalin-6-yl)-3-(2- methoxyethyl)urea |
| ADG 1-206 | 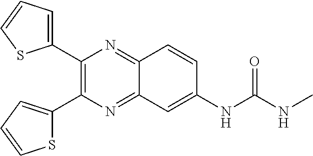 [SMILES: CNC(=O)Nc1ccc2nc(-c3cccs3)c(-c3cccs3)nc2c1] | 1-(2,3-di(thiophen-2- yl)quinoxalin-6-yl)3- methylurea |
| ADG 1-207 | 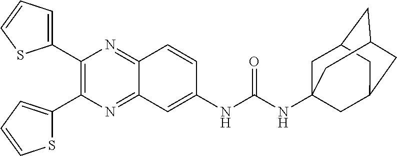 [SMILES: O=C(Nc1ccc2nc(-c3cccs3)c(-c3cccs3)nc2c1)NC12CC3CC(CC(C3)C1)C2] | 1-((1S,3s)-adamantan- l-yl)-3-(2,3- di(thiophen-2-yl) quinoxalin-6-yl)urea |

Table 19 demonstrates the protocol information for in vitro and in vivo pharmacokinetics performed for each of the represented compounds. Table 20 provides results of in vitro and in vivo pharmacokinetics performed for each of the represented compounds.

TABLE 19

| Assay # | Assay Description | Host | Dosage | | Timing | | Control used |
|---|---|---|---|---|---|---|---|
| 1 | Turbidity-based aqueus solubility- | In vitro | na | | na | | na |
| 2 | Protein binding via Rapid Equilibrium | Rat (SD) | 2 | μM | na | | Chlorpromazine |
| | Dialysis- brain | | | | | | |
| 3 | Protein binding via Rapid Equilibrium | Rat (SD) | 2 | μM | na | | Warfarin |
| | Dialysis- plasma | | | | | | |
| 4 | MDCKII-MDR1 Permeability | | | | | | |
| 5 | Microsomal stability assay (half-life) | Human | 2 | μM | na | | 7-Ethoxycoumarin |
| 6 | Microsomal stability assay (half-life) | Mouse (CD1) | 2 | μM | na | | 7-Ethoxycoumarin |
| 7 | Microsomal stability assay (half-life) | Rat (SD) | 2 | μM | na | | 7-Ethoxycoumarin |
| 8 | In vivo PK, oral gavage, plasma | Rat (SD) | 5 | mg/kg | 0.25 | hr | na |
| 9 | In vivo PK, oral gavage, plasma | Rat (SD) | 5 | mg/kg | 0.5 | hr | na |
| 10 | In vivo PK, oral gavage, plasma | Rat (SD) | 5 | mg/kg | 1 | hr | na |
| 11 | In vivo PK, oral gavage, plasma | Rat (SD) | 5 | mg/kg | 4 | hr | na |
| 12 | In vivo PK, oral gavage, plasma | Rat (SD) | 5 | mg/kg | 8 | hr | na |
| 13 | In vivo PK, oral gavage, plasma | Rat (SD) | 5 | mg/kg | 24 | hr | na |
| 14 | In vivo PK, IV dose, Terminal- plasma | Rat (SD) | 1 | mg/kg | 0.083 | hr | na |
| 15 | In vivo PK, IV dose, Terminal- plasma | Rat (SD) | 1 | mg/kg | 1 | hr | na |
| 16 | In vivo PK, IV dose, Terminal- plasma | Rat (SD) | 1 | mg/kg | 4 | hr | na |
| 17 | In vivo PK, IV dose, Terminal- plasma | Rat (SD) | 1 | mg/kg | 8 | hr | na |
| 18 | In vivo PK, IV dose, Terminal- plasma | Rat (SD) | 1 | mg/kg | 24 | hr | na |
| 19 | In vivo PK, IV dose, Terminal- brain | Rat (SD) | 1 | mg/kg | 0.083 | hr | na |
| 20 | In vivo PK, IV dose, Terminal- brain | Rat (SD) | 1 | mg/kg | 1 | hr | na |
| 21 | In vivo PK, IV dose, Terminal- brain | Rat (SD) | 1 | mg/kg | 4 | hr | na |
| 22 | In vivo PK, IV dose, Terminal- brain | Rat (SD) | 1 | mg/kg | 8 | hr | na |
| 23 | In vivo PK, IV dose, Terminal- brain | Rat (SD) | 1 | mg/kg | 24 | hr | na |

| 24 | Western blot- Differentiated CAD cell | In vitro | DMSO | | na | DMSO |
|---|---|---|---|---|---|---|
| | lysates- H3K9ac levels normalized to | | | | | |
| | H3 and DMSO control | | | | | |
| 25 | Western blot- Differentiated CAD cell | In vitro | 1 | uM | na | DMSO |
| | lysates- H3K9ac levels normalized to | | | | | |
| | H3 and DMSO control | | | | | |
| 26 | Western blot- Differentiated CAD cell | In vitro | 5 | uM | na | DMSO |
| | lysates- H3K9ac levels normalized to | | | | | |
| | H3 and DMSO control | | | | | |
| 27 | Western blot- Differentiated CAD cell | In vitro | 10 | uM | na | DMSO |
| | lysates- H3K9ac levels normalized to | | | | | |
| | H3 and DMSO control | | | | | |
| 28 | Western blot- Differentiated CAD cell | In vitro | 20 | uM | na | DMSO |
| | lysates- H3K9ac levels normalized to | | | | | |
| | H3 and DMSO control | | | | | |
| 29 | Western blot- Differentiated CAD cell | In vitro | 50 | uM | na | DMSO |
| | lysates- H3K9ac levels normalized to | | | | | |
| | H3 and DMSO control | | | | | |
| 30 | Western blot- Differentiated CAD cell | In vitro | 100 | uM | na | DMSO |
| | lysates- H3K9ac levels normalized to | | | | | |
| | H3 and DMSO control | | | | | |

TABLE 20

| Assay | | | | | | |
|---|---|---|---|---|---|---|
| # | ADG-204 | ADG-205c | ADG-206 | ADG-207 | control | Units |
| 1 | 2 | 5 | 5 | 5 | na | highest soluble |
| | | | | | | concentration, μM |
| 2 | 0.05328662 | | 0.79462814 | 0.89939826 | 0.64754077 | % Protein free |
| 3 | 0.19935557 | 0.29145546 | 0.65651465 | 0.94281121 | 0.51774484 | % Protein free |
| 4 | | | | | | |
| 5 | 69.5 | 27.8 | 43.3 | 18.7 | 7.97 | T1/2 (min) |
| 6 | 301 | 4.72 | 4.39 | 5.5 | <15 | T1/2 (min) |
| 7 | 235 | 11.6 | 6.36 | 5.5 | 6.13 | T1/2 (min) |
| 8 | BQL | 83.4666667 | 1.47 | BQL | na | ng/mL |
| 9 | BQL | 370.666667 | 2.31666667 | BQL | na | ng/mL |
| 10 | BQL | 640.666667 | 7.13666667 | BQL | na | ng/mL |
| 11 | BQL | 692.666667 | 12.76 | BQL | na | ng/mL |
| 12 | BQL | 354.333333 | 12.245 | BQL | na | ng/mL |
| 13 | BQL | 3.06 | BQL | BQL | na | ng/mL |
| 14 | 1910 | 1630 | 1090 | 1660 | na | ng/mL |
| 15 | 220 | 1040 | 165 | 54.5 | na | ng/mL |
| 16 | 4.49 | 140 | 3 | 1.37 | na | ng/mL |
| 17 | 4.27 | 29.1 | 4.98 | 1.05 | na | ng/mL |
| 18 | 1.52 | 2.63 | BQL | BQL | na | ng/mL |
| 19 | 487 | 415 | 862 | 192 | na | ng/g |
| 20 | 109 | 135 | 66.9 | 50.8 | na | ng/g |
| 21 | BQL | 23.4 | BQL | BQL | na | ng/g |
| 22 | BQL | BQL | BQL | BQL | na | ng/g |
| 23 | BQL | BQL | BQL | BQL | na | ng/g |
| 24 | 100.00% | 100.00% | 100.00% | 100.00% | na | na |
| 25 | 22.30% | 21.62% | 46.94% | 57.45% | na | na |
| 26 | 13.48% | 17.18% | 35.39% | 63.69% | na | na |
| 27 | 8.32% | 14.26% | 19.49% | 72.97% | na | na |
| 28 | 17.03% | 21.52% | 8.45% | 70.92% | na | na |
| 29 | 9.64% | 29.67% | 5.83% | 116.85% | na | na |
| 30 | 24.00% | 16.42% | 1.24% | 107.80% | na | na |

Table 2 depicts the compounds' properties

TABLE 2

| | | | | Molecular | |
|---|---|---|---|---|---|
| | Parent- | Lot | Exact | Formula of | Stock |
| Compound | MW | MW | Mass | Free Base | Solvent |
| ADG I-204 | 422.56 | 422.56 | 422.1235 | C22H22N4Os2 | DMSO |
| ADG I-205 | 410.51 | 410.51 | 410.0871 | C20H18N4O2S2 | DMSO |
| ADG I-206 | 366.46 | 366.46 | 366.0609 | C18H14N4Os2 | DMSO |
| ADG I-207 | 486.65 | 486.65 | 486.1548 | C27H26N4OS2 | DMSO |

Brain Availability

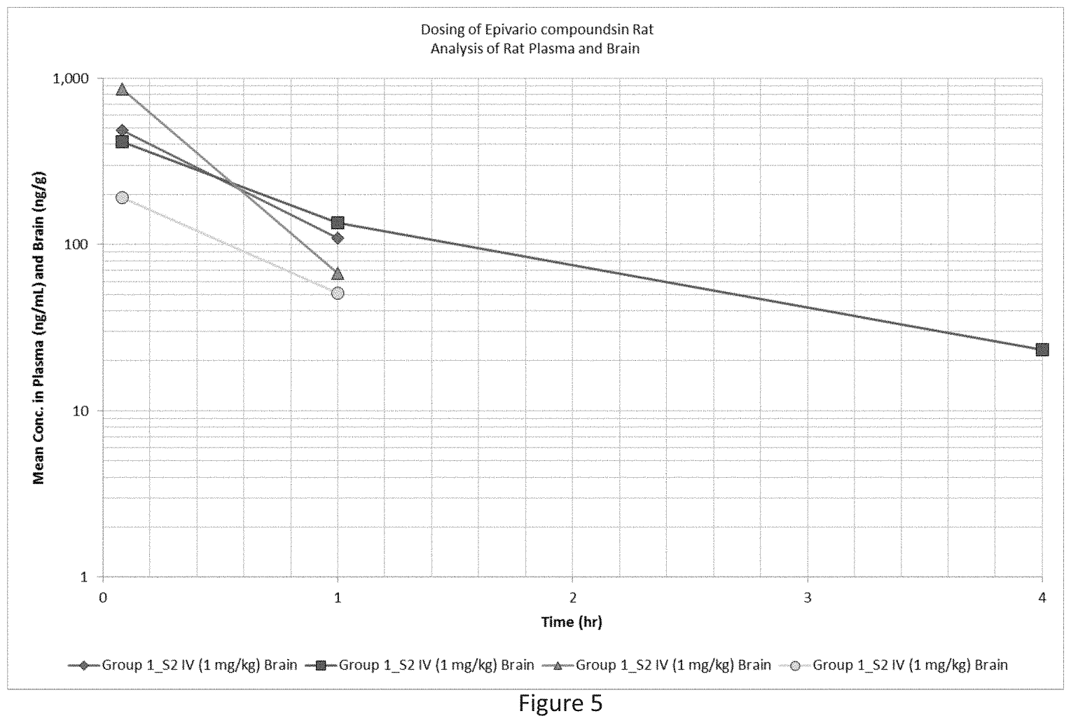

Table 3 and FIG. 5 depict the brain availability of ADG I-204, ADG I-205, ADG I-206, and ADG I-207 after IV administration of 1 mg/kg dose.

TABLE 3

Collection time point
(mean value for 3 animals, values in ng/g)

| Compound | 0.083 hr | 1 hr | 4 hr | 8 hr | 24 hr |
|---|---|---|---|---|---|
| ADG-204 | 487 | 109 | BQL | BQL | BQL |
| ADG-205c | 415 | 135 | 23.4 | BQL | BQL |
| ADG-206 | 862 | 66.9 | BQL | BQL | BQL |
| ADG-207 | 192 | 50.8 | BQL | BQL | BQL |

BQL = Below Quantitation Limit (1.00 ng/mL)

### ADG I-204 Pharmacokinetics

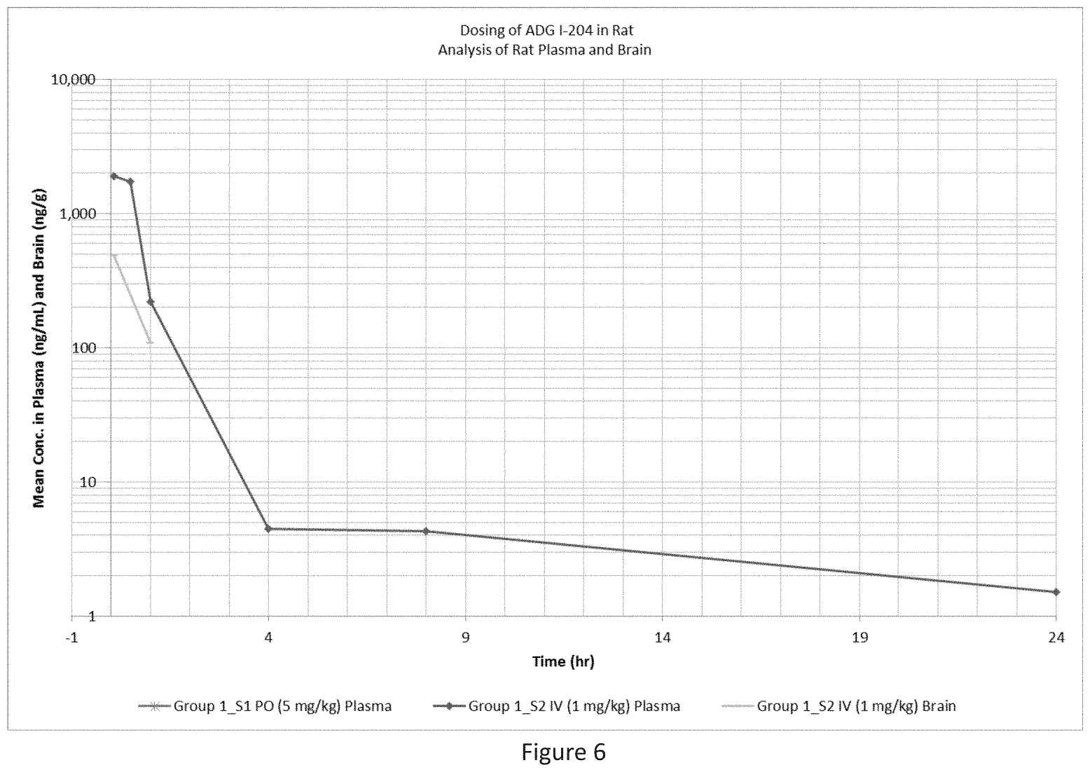

Tables 4 and 5 depict the summary of rat plasma sample concentrations after administration of ADG I-204. Table 6 depict the summary of rat brain sample concentrations after administration of ADG I-204. FIG. 6 provides a summary of rat plasma and brain concentrations.

TABLE 4

| Group 1_S1 | ADG I-204 Concentrations | | | | | |
|---|---|---|---|---|---|---|
| PO (5 mg/kg) | (ng/mL) in Rat Plasma | | | | | |
| Time Points (hrs) | Animal ID | | | | | |
| Day 1 | 1 | 2 | 3 | Mean | SD | % CV |
| 0.250 | BQL | BQL | BQL | NA | NA | NA |
| 0.500 | BQL | BQL | BQL | NA | NA | NA |
| 1.00 | BQL | BQL | BQL | NA | NA | NA |
| 4.00 | BQL | BQL | BQL | NA | NA | NA |
| 8.00 | BQL | BQL | BQL | NA | NA | NA |
| 24.0 | BQL | BQL | BQL | NA | NA | NA |

BQL = Below Quantitation Limit (1.00 ng/mL)
NA = Not Applicable

TABLE 5

| Group 1_S2 | ADG I-204 Concentrations | | | | | |
|---|---|---|---|---|---|---|
| IV (1 mg/kg) | (ng/mL) in Rat Plasma | | | | | |
| Time Points (hrs) | Terminal | | | | | |
| Day 2 | * | * | * | Mean | SD | % CV |
| 0.0830 | 2220 | 2340 | 1170*** | 1910 | 644 | 33.7% |
| 0.500^{†} | 4190** | 357 | 634 | 1730 | 2140 | 123.7% |
| 1.00 | 278 | 103 | 279 | 220 | 101 | 45.9% |
| 4.00 | 8.63 | 4.35 | NS | 4 | NA | NA |
| 8.00 | 3.92 | 2.97 | 5.93 | 4 | 2 | 354% |
| 24.0 | 1.95 | 1.09 | BQL | 2 | NA | NA |

* Each cell reflects an individual animal for terminal time points.
**Verified vial position and calculated value
***Animal 3 in Session 2 only received 70% of the test article
^{†}Not a terminal time point. Includes animals 4, 5 and 6.
BQL = Below Quantitation Limit (1.00 ng/mL)
NS = No Sample Received
NA = Not Applicable

TABLE 6

| Group 1_S2 | ADG I-204 Concentrations | | | | | |
|---|---|---|---|---|---|---|
| IV (1 mg/kg) | (ng/g) in Rat Brain | | | | | |
| Time Points (hrs) | Terminal | | | | | |
| Day 2 | * | * | * | Mean | SD | % CV |
| 0.0830 | 637 | 583 | 242*** | 487 | 214 | 43.9% |
| 1.00 | 119 | 60.3 | 149 | 109 | 45 | 41.4% |
| 4.00 | BQL | BQL | NS | NA | NA | NA |
| 8.00 | BQL | BQL | BQL | NA | NA | NA |
| 24.0 | BQL | BQL | BQL | NA | NA | NA |

* Each cell reflects an individual animal for terminal time points.
***Animal 3 in Session 2 only received 70% of the test article
BQL = Below Quantitation Limit (10.0 ng/g)
NS = No Sample Received
NA = Not Applicable

ADG I-205 Pharmacokinetics

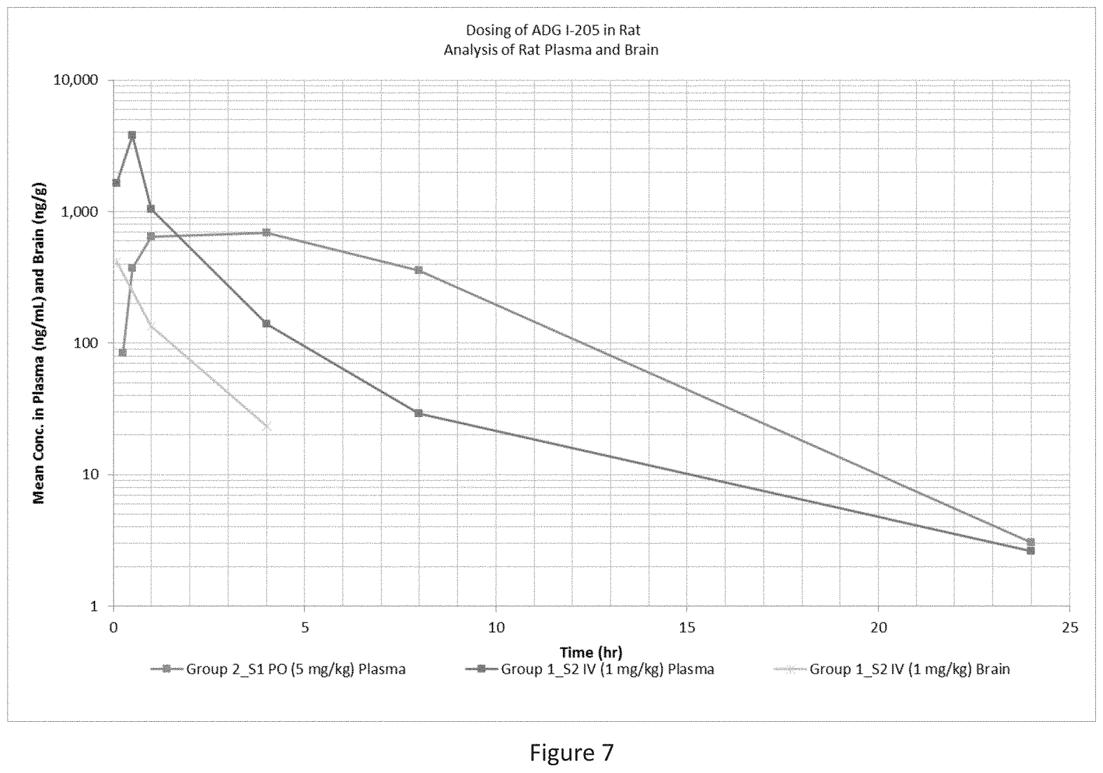

Tables 7 and 8 depict the summary of rat plasma sample concentrations after administration of ADG I-204. Table 9 depict the summary of rat brain sample concentrations after administration of ADG I-204. FIG. 7 provides a summary of rat plasma and brain concentrations.

TABLE 7

| Group 2_S1 | ADG I-205 Concentrations | | | | | |
|---|---|---|---|---|---|---|
| PO (5 mg/kg) | (ng/mL) in Rat Plasma | | | | | |
| Time Points (hrs) | Animal ID | | | | | |
| Day 1 | 4 | 5 | 6 | Mean | SD | % CV |
| 0.250 | 30.4 | 73.0 | 147 | NA | NA | NA |
| 0.500 | 145 | 298 | 669 | NA | NA | NA |
| 1.00 | 466 | 550 | 906 | NA | NA | NA |
| 4.00 | 710 | 430 | 938 | NA | NA | NA |
| 8.00 | 531 | 235 | 297 | NA | NA | NA |
| 24.0 | 3.51 | 1.48 | 4.19 | NA | NA | NA |

NA = Not Applicable

TABLE 8

| Group 1_S2 | ADG I-205 Concentrations | | | | | |
|---|---|---|---|---|---|---|
| IV (1 mg/kg) | (ng/mL) in Rat Plasma | | | | | |
| Time Points (hrs) | Terminal | | | | | |
| Day 2 | * | * | * | Mean | SD | % CV |
| 0.0830 | 2310 | 1700 | 865*** | 1630 | 725 | 44.5% |
| 0.500^{†} | 8520** | 1380 | 1470 | 3790 | 4100 | 108.2% |
| 1.00 | 1030 | 895 | 1190 | 1040 | 148 | 14.2% |
| 4.00 | 187 | 92.3 | NS | 140 | NA | NA |
| 8.00 | 28.5 | 27.0 | 31.9 | 29 | 3 | 8.6% |
| 24.0 | BQL | 2.63 | BQL | 3 | NA | NA |

* Each cell reflects an individual animal for terminal time points.
**Verified vial position and calculated value
***Animal 3 in Session 2 only received 70% of the test article
^{†}Not a terminal time point. Includes animals 4, 5 and 6.
BQL = Below Quantitation Limit (2.00 ng/mL)
NS = No Sample Received
NA = Not Applicable

TABLE 9

| Group 1_S2 | ADG I-205 Concentrations | | | | | |
|---|---|---|---|---|---|---|
| IV (1 mg/kg) | (ng/g) in Rat Brain | | | | | |
| Time Points (hrs) | Terminal | | | | | |
| Day 2 | * | * | * | Mean | SD | % CV |
| 0.0830 | 544 | 487 | 213*** | 415 | 177 | 42.7% |
| 1.00 | 132 | 102 | 171 | 135 | 35 | 25.6% |
| 4.00 | 23.4 | BQL | NS | 23 | NA | NA |
| 8.00 | BQL | BQL | BQL | NA | NA | NA |
| 24.0 | BQL | BQL | BQL | NA | NA | NA |

* Each cell reflects an individual animal for terminal time points
***Animal 3 in Session 2 only received 70% of the test article
BQL = Below Quantitation Limit (20.0 ng/g)
NS = No Sample Received
NA = Not Applicable

ADG I-206 Pharmacokinetics

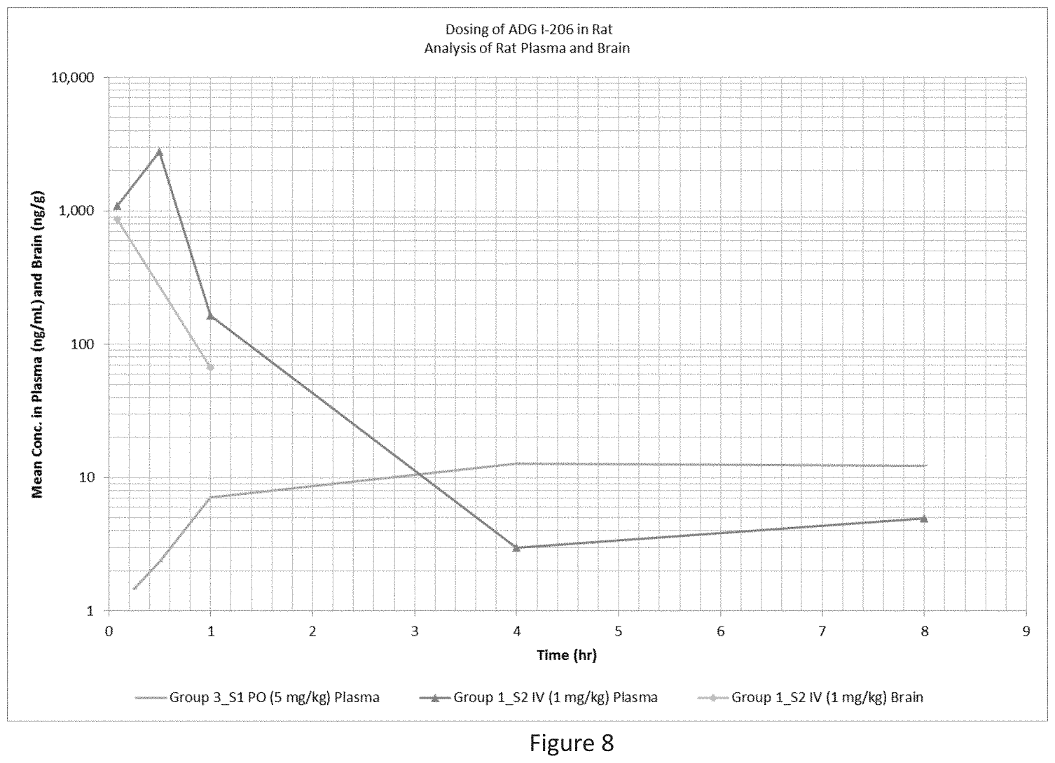

Tables 10 and 11 depict the summary of rat plasma sample concentrations after administration of ADG I-204. Table 12 depict the summary of rat brain sample concentrations after administration of ADG I-204. FIG. 8 provides a summary of rat plasma and brain concentrations.

TABLE 10

| Group 3_S1 | ADG I-206 Concentrations | | | | | |
|---|---|---|---|---|---|---|
| PO (5 mg/kg) | (ng/mL) in Rat Plasma | | | | | |
| Time Points (hrs) | Animal ID | | | | | |
| Day 1 | 7 | 8 | 9 | Mean | SD | % CV |
| 0.250 | 1.47 | BQL | BQL | NA | NA | NA |
| 0.500 | 2.86 | 2.16 | 1.93 | NA | NA | NA |
| 1.00 | 5.47 | 9.00 | 6.94 | NA | NA | NA |
| 4.00 | 17.2 | 8.32 | NS | NA | NA | NA |
| 8.00 | 15.6 | 8.89 | NS | NA | NA | NA |
| 24.0 | BQL | BQL | NS | NA | NA | NA |

BQL = Below Quantitation Limit (1.00 ng/mL)
NS = No Sample Received
NA = Not Applicable

TABLE 11

| Group 1_S2 | ADG I-206 Concentrations | | | | | |
|---|---|---|---|---|---|---|
| IV (1 mg/kg) | (ng/mL) in Rat Plasma | | | | | |
| Time Points (hrs) | Terminal | | | | | |
| Day 2 | * | * | * | Mean | SD | % CV |
| 0.0830 | 1500 | 1160 | 610*** | 1090 | 449 | 41.2% |
| 0.500^{†} | 7310** | 527 | 507 | 2780 | 3920 | 141.0% |
| 1.00 | 176 | 177 | 142 | 165 | 20 | 12.1% |
| 4.00 | 4.19 | 1.81 | NS | 3 | NA | NA |
| 8.00 | BQL | 1.44 | 8.52 | 5 | NA | NA |
| 24.0 | BQL | BQL | BQL | NA | NA | NA |

* Each cell reflects an individual animal for terminal time points.
**Verified vial position and calculated value
***Animal 3 in Session 2 only received 70% of the test article
^{†}Not a terminal time point. Includes animals 4, 5 and 6,
BQL = Below Quantitation Limit (1.00 ng/mL)
NS = No Sample Received
NA = Not Applicable

TABLE 12

| Group 1_S2 | ADG I-206 Concentrations | | | | | |
|---|---|---|---|---|---|---|
| IV (1 mg/kg) | (ng/g) in Rat Brain | | | | | |
| Time Points (hrs) | Terminal | | | | | |
| Day 2 | * | * | * | Mean | SD | % CV |
| 0.0830 | 1080 | 1050 | 455*** | 862 | 353 | 41.0% |
| 1.00 | 68.8 | 57.6 | 74.3 | 67 | 9 | 12.7% |
| 4.00 | BQL | BQL | NS | NA | NA | NA |
| 8.00 | BQL | BQL | BQL | NA | NA | NA |
| 24.0 | BQL | BQL | BQL | NA | NA | NA |

* Each cell reflects an individual animal for terminal time points.
***Animal 3 in Session 2 only received 70% of the test article
BQL = Below Quantitation Limit (10.0 ng/g)
NS = No Sample Received
NA = Not Applicable

ADG I-207 Pharmacokinetics

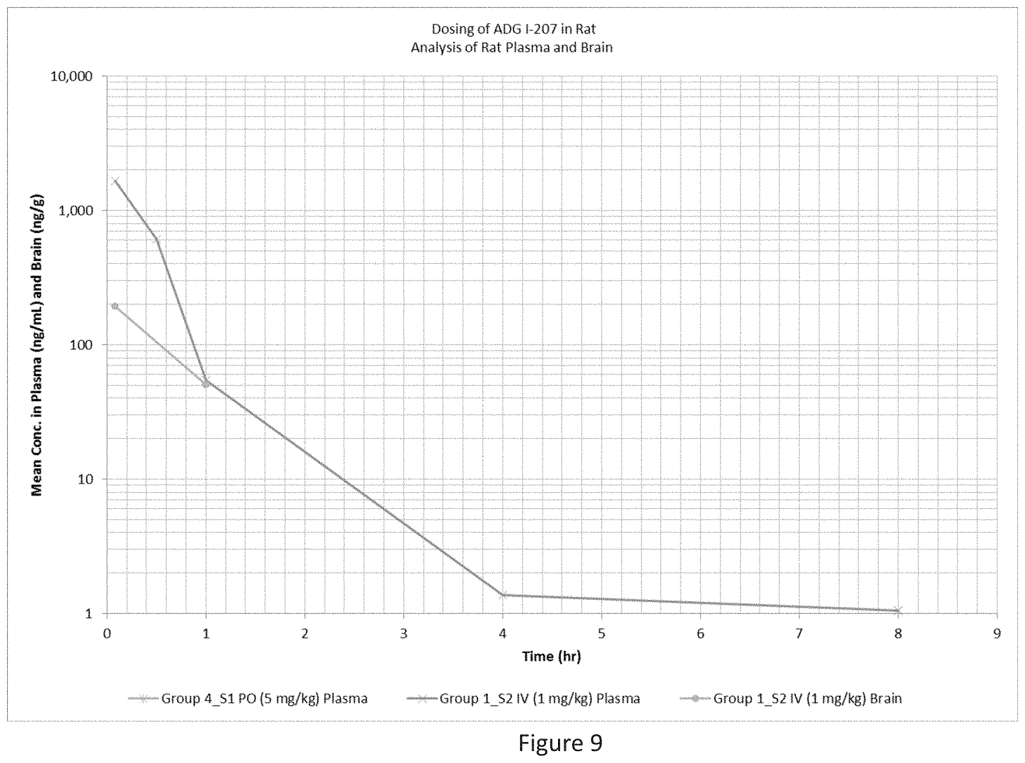

Tables 13 and 14 depict the summary of rat plasma sample concentrations after administration of ADG I-204. Table 15 depict the summary of rat brain sample concentrations after administration of ADG I-204. FIG. 9 provides a summary of rat plasma and brain concentrations.

TABLE 13

| Group 4_S1 | AUG I-207 Concentrations | | | | | |
|---|---|---|---|---|---|---|
| PO (5 mg/kg) | (ng/mL) in Rat Plasma | | | | | |
| Time Points (krs) | Animal ID | | | | | |
| Day 1 | 10 | 11 | 12 | Mean | SD | % CV |
| 0.250 | BQL | BQL | BQL | NA | NA | NA |
| 0.500 | BQL | BQL | BQL | NA | NA | NA |
| 1.00 | BQL | BQL | BQL | NA | NA | NA |
| 4.00 | BQL | BQL | BQL | NA | NA | NA |
| 8.00 | BQL | BQL | BQL | NA | NA | NA |
| 24.0 | 1.18** | BQL | BQL | NA | NA | NA |

**Verified vial position and calculated value
BQL = Below Quantitation Limit (1.00 ng/mL)
NA = Not Applicable

TABLE 14

| Group 1_S2 | ADG I-207 Concentrations | | | | | |
|---|---|---|---|---|---|---|
| IV (1 mg/kg) | (ng/mL) in Rat Plasma | | | | | |
| Time Points (hrs) | Terminal | | | | | |
| Day 2 | * | * | * | Mean | SD | % CV |
| 0.0830 | 1640 | 2230 | 1110 | 1660*** | 560 | 33.7% |
| 0.500^{†} | 1560** | 93.3 | 170 | 608 | 826 | 135.9% |
| 1.00 | 70.3 | 27.3 | 65.8 | 55 | 24 | 43.3% |
| 4.00 | BQL | 1.37 | NS | 1 | NA | NA |
| 8.00 | BQL | 1.00 | 1.09 | 1 | NA | NA |
| 24.0 | BQL | BQL | BQL | NA | NA | NA |

* Each cell reflects an individual animal for terminal time points.
**Verified vial position and calculated value
***Animal 3 in Session 2 only received 70% of the test article
^{†}Not a terminal time point. Includes animals 4, 5 and 6.
BQL = Below Quantitation Limit (1.00 ng/mL)
NS = No Sample Received
NA = Not Applicable

TABLE 15

| Group 1_S2 | ADG I-207 Concentrations | | | | | |
|---|---|---|---|---|---|---|
| IV (1 mg/kg) | (ng/g) in Rat Brain | | | | | |
| Time Points (hrs) | Terminal | | | | | |
| Day 2 | * | * | * | Mean | SD | % CV |
| 0.0830 | 204 | 253 | 119*** | 192 | 67.8 | 35.3% |
| 1.00 | 53.6 | 39.8 | 58.9 | 51 | 9.9 | 19.4% |
| 4.00 | BQL | BQL | NS | NA | NA | NA |
| 8.00 | BQL | BQL | BQL | NA | NA | NA |
| 24.0 | BQL | BQL | BQL | NA | NA | NA |

* Each cell reflects an individual animal for terminal time points.
***Animal 3 in Session 2 only received 70% of the test article
BQL = Below Quantitation Limit (20.0 ng/g)
NS = No Sample Received
NA = Not Applicable

### Example 6: Assessment of Extracellular Acetate Derived Acetyl in Histone Acetylation

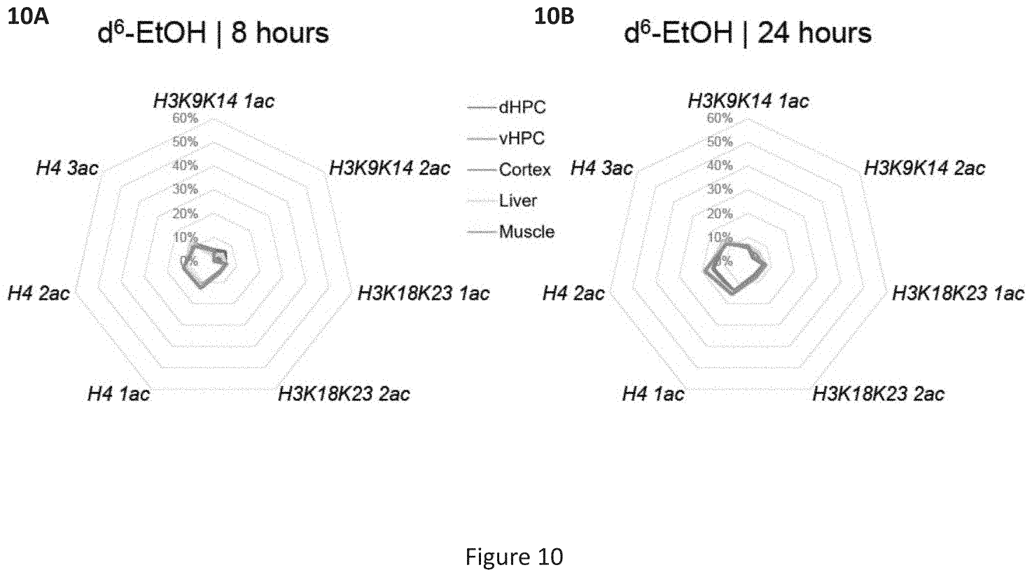

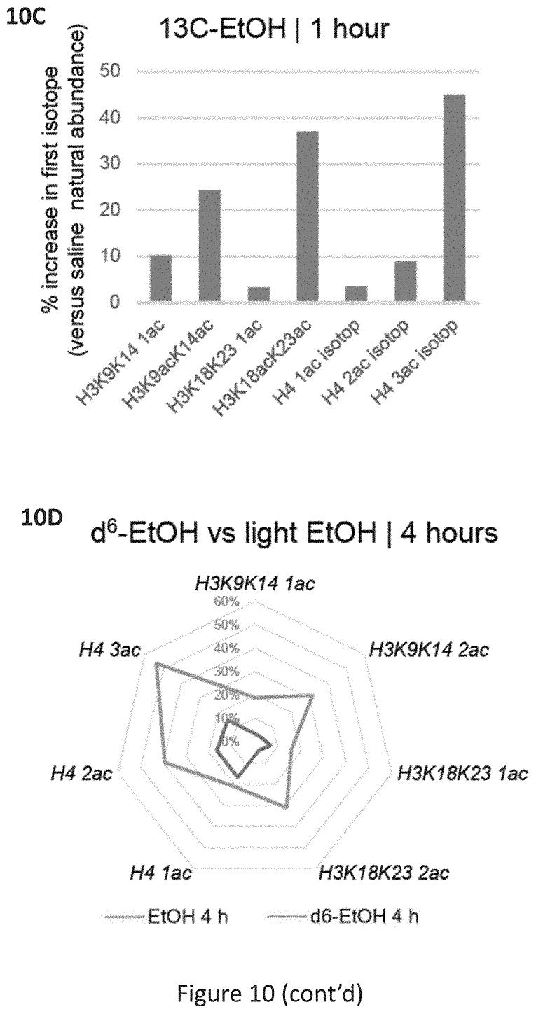

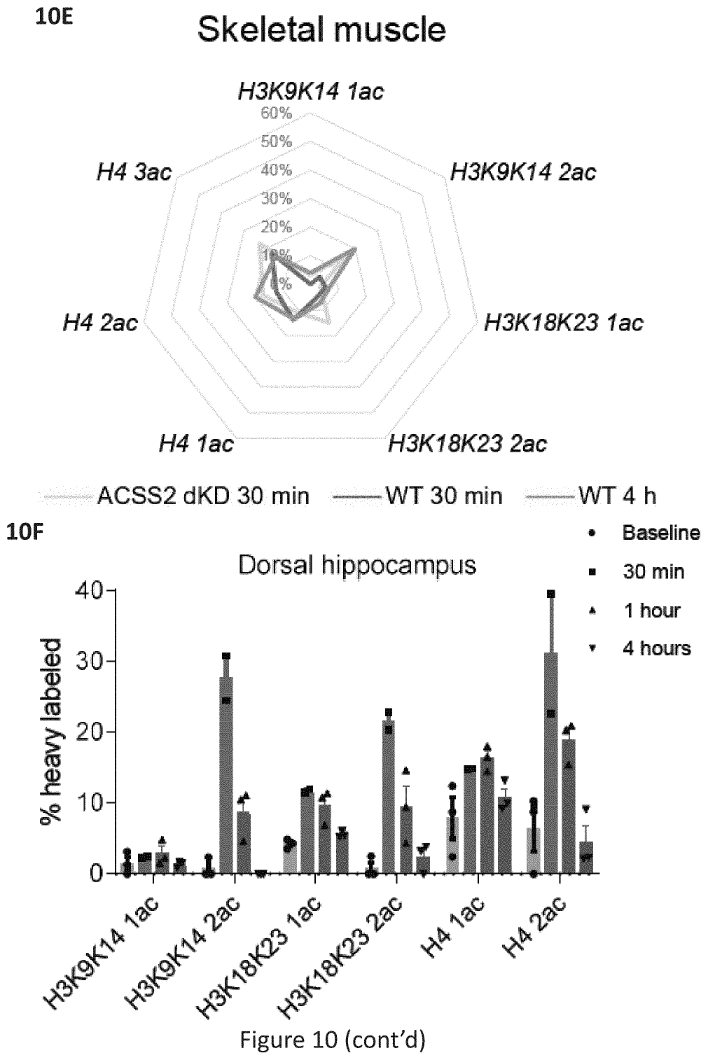

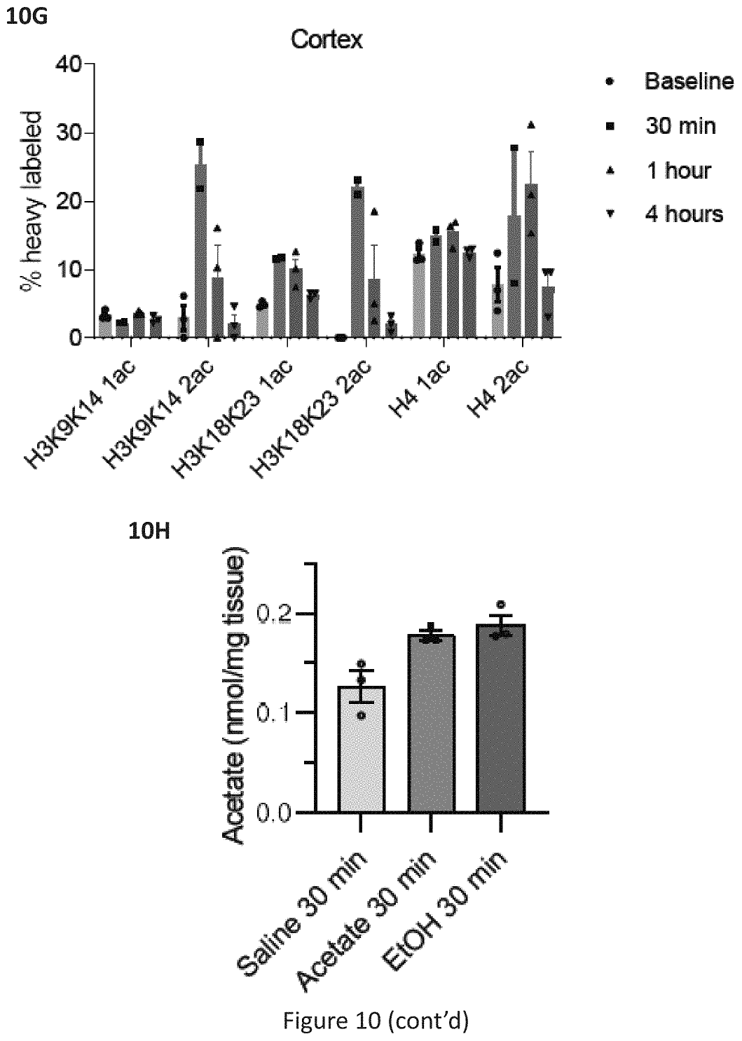

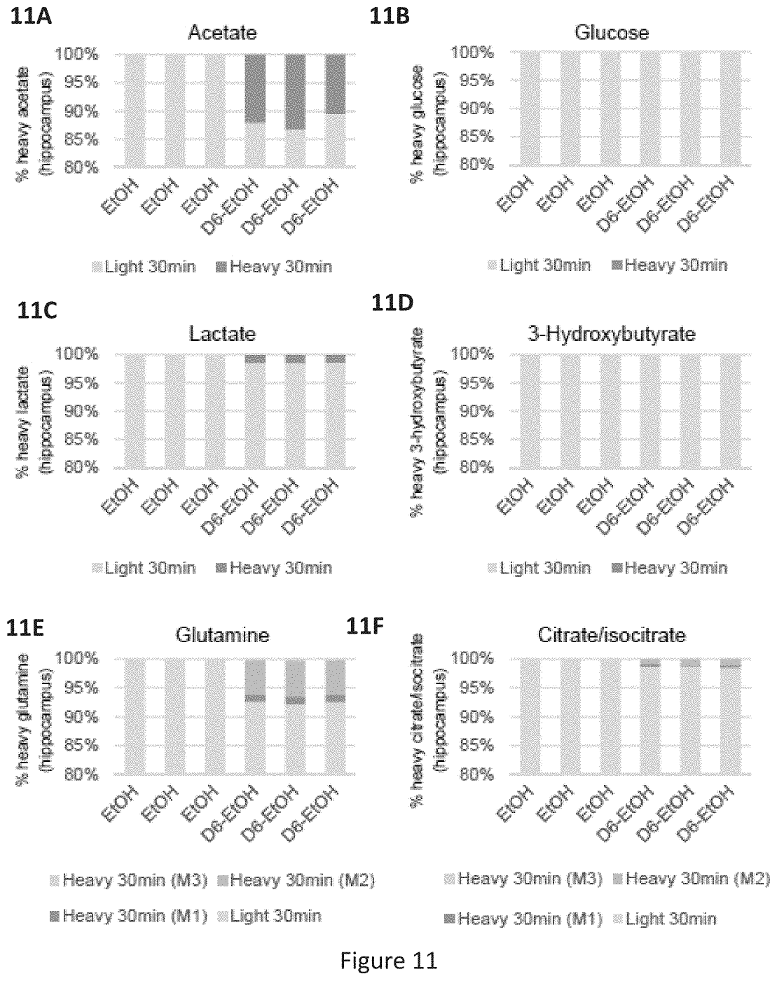

Mice were intraperitoneally injected with 2 g/kg deuterated acetate (d3-acetate). Thereafter, rapid label incorporation into brain histone acetylation was detected, at similar levels in both hippocampus and cortex (FIG. 10F-10G). Relative labeling was highest at 30 minutes and returned to background levels at 4 hours post-injection, indicating rapid incorporation of acetate-derived acetyl groups as well as rapid turnover of brain histone acetylation. Notably, acetate levels in the hippocampus were significantly increased at 30 minutes after alcohol injection, or following acetate injection (FIG. 10H), and detected substantial amounts of heavy acetate in the hippocampus as early as 30 minutes following injection with d6-EtOH (FIG. 11A).

1. Level of Alcohol-Derived Carbons Incorporated into Other Key Metabolites in Hippocampal Tissue

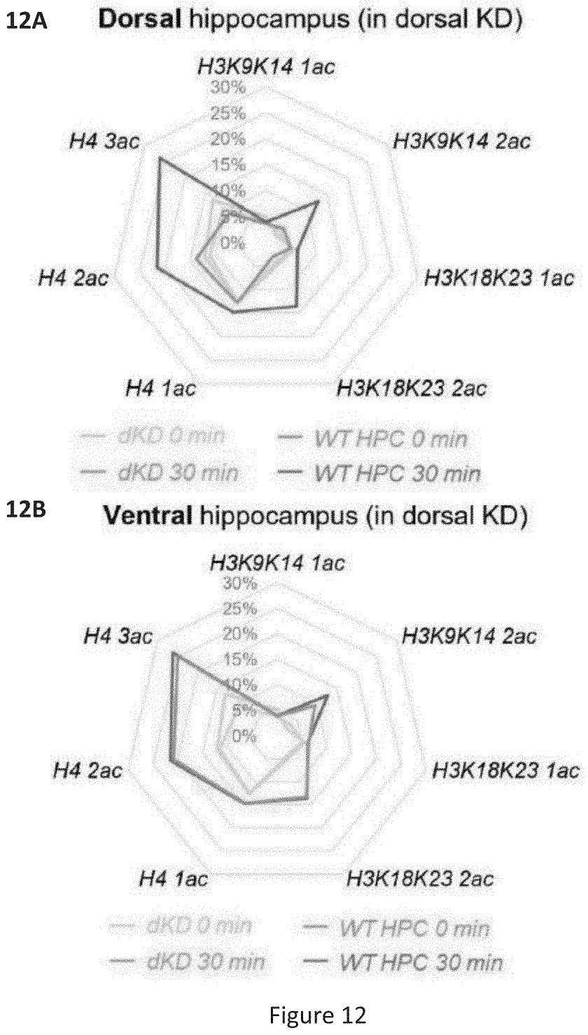

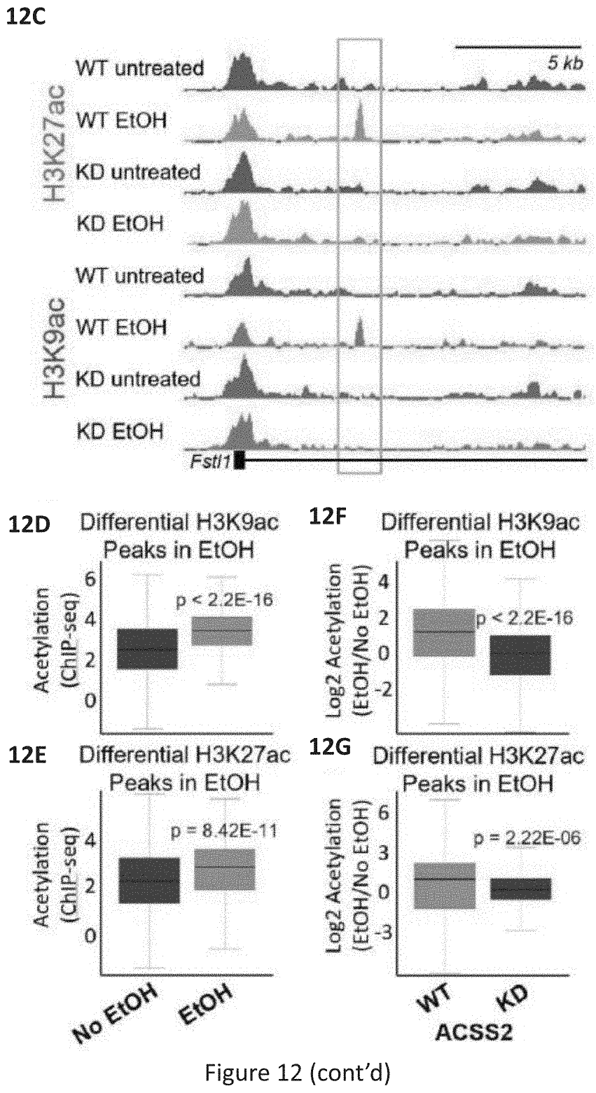

While no label incorporation into glucose and 3-hydroxybutyrate was detected, and only a fraction into lactate pools (<1%), alcohol labels were detected in glutamine pools in the hippocampus (FIG. 11B-11E). In the brain, de novo synthesis of glutamine occurs in astrocytes and replenishes the glutamate-glutamine cycle, as it is trafficked into glutamatergic neurons for production of the neurotransmitter glutamate. Citrate—the substrate used by ATP-citrate lyase (ACL) to produce nucleo-cytoplasmic acetyl-CoA—is generated from α-ketoglutarate that can derive from carboxylation of glutamine; this path could provide another route for alcohol to contribute to histone acetylation. However, only traces of alcohol-derived label in hippocampal citrate/isocitrate pools were detected (FIG. 11F). Taken together with the mass spec in ACSS2 KD animals as shown in FIGS. 12A and 12B, these results show alcohol-derived acetate contributing to hippocampal histone acetylation, converted directly by ACSS2. Accordingly, the data suggests that increased blood acetate from alcohol metabolism promotes ACSS2-mediated dynamic histone acetylation in the brain.

2. Functional Relevance of Alcohol-Derived Acetate for ACSS2-Dependent Histone Acetylation in Regulating Hippocampal Gene Expression

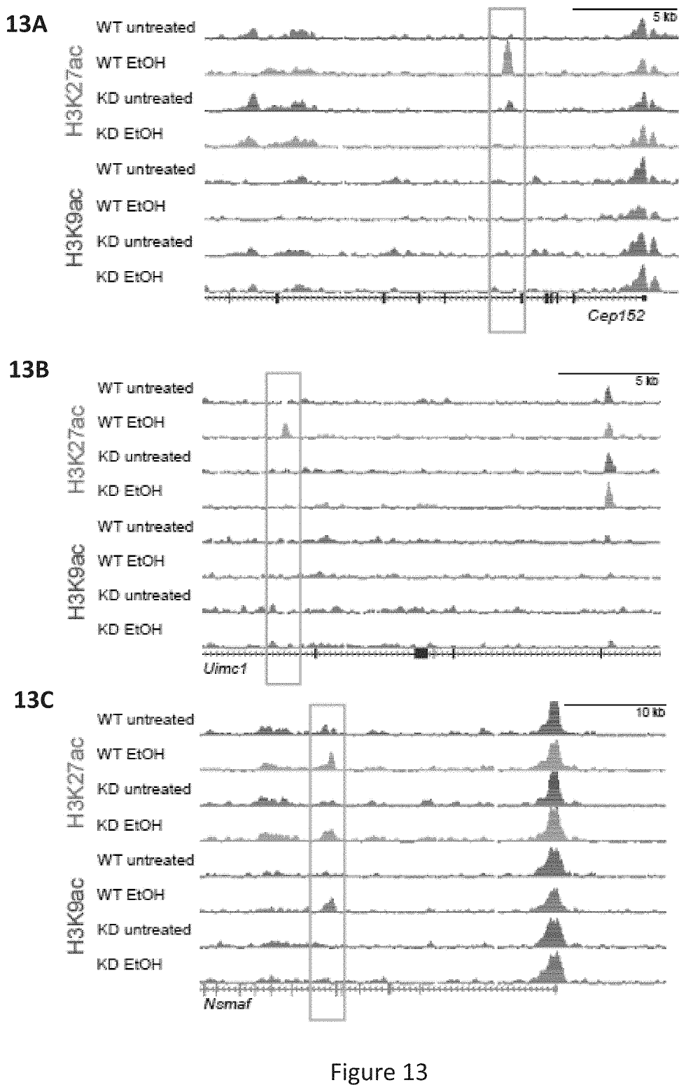

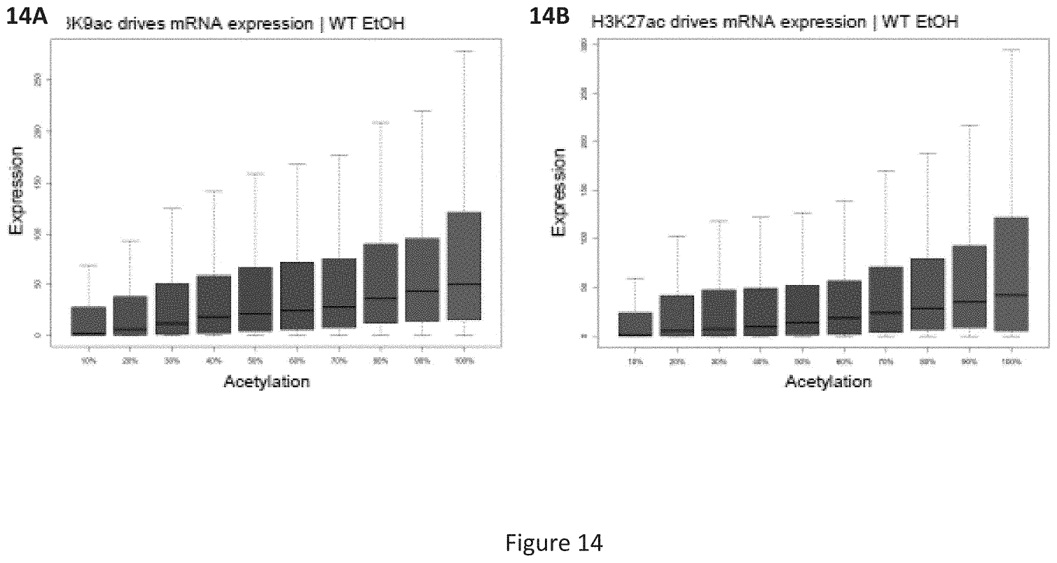

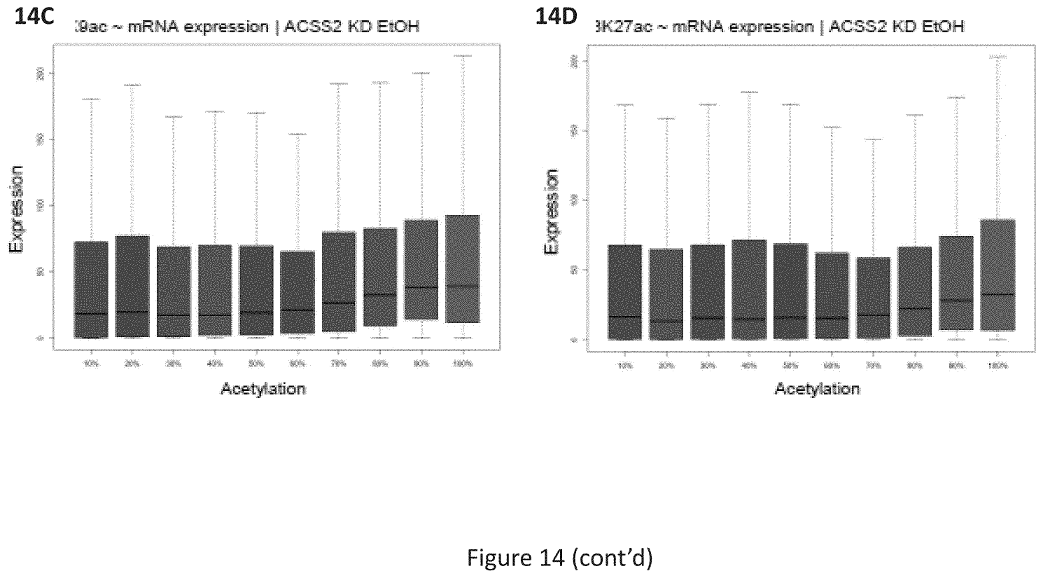

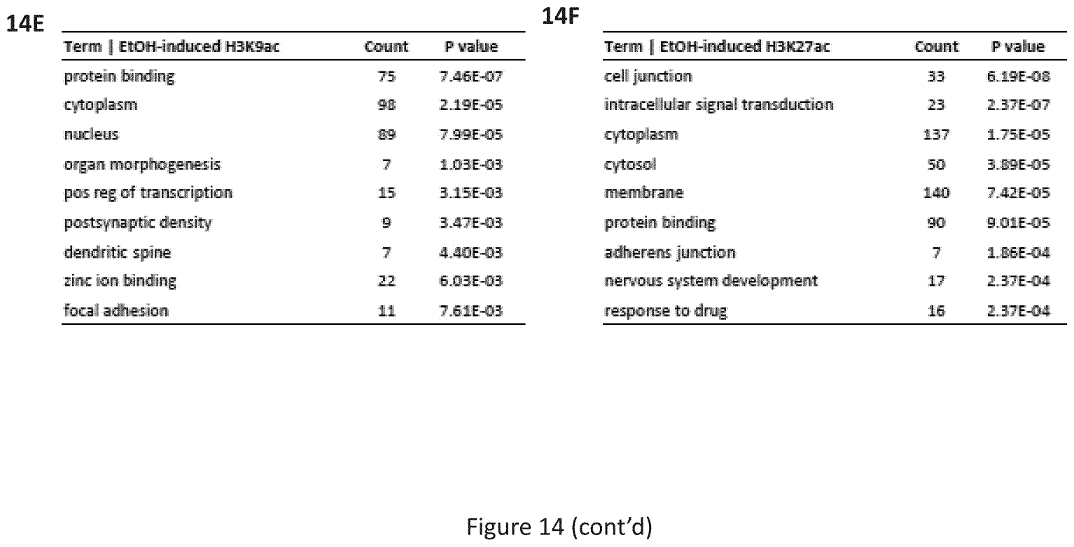

Alcohol administration in WT mice was shown to result in significant enrichment of H3K9ac and H3K27ac peaks at key neuronal genes and genome-wide, and this enrichment was greatly attenuated in the ACSS2 KD (FIGS. 12C-12G; ChIP-seq performed 1 hour after alcohol injection). For example, ACSS2-dependent and alcohol-induced histone acetylation at FstlI (follistatin-like 1: FIG. 12C), a neuronal gene that has been implicated in neuronal development and migration. Alcohol-induced H3K27ac at Cep152 (centrosomal protein of 152 kDa) gene FIG. 13A) was observed, an important regulator of genome integrity that is recurrently mutated in intellectual developmental disorders and microcephaly. Another example is the Uimc1 (ubiquitin interaction motif containing 1) gene (FIG. 13B), previously connected to neurodevelopmental disorders and autism. Evaluating the histone acetylation ChIP-Seq genome-wide, 74% of H3K9ac peaks changed upon alcohol exposure were increased (339 out of 458 changed peaks called with MACS2, using 10% FDR significance threshold for DiffBind; FIG. 12D), and that 60% of differential H3K27ac peaks were increased by ethanol (490 out of 816 peaks, FIG. 12E; ChIP-seq performed 1 hour after alcohol injection). Strikingly, this response was eliminated in ACSS2 KD animals—98% of H3K9ac and H3K27ac peaks increased in WT failed to induce upon EtOH treatment in the dHPC (FIG. 12F-12G). RNA-seq was performed to characterize the transcriptional response and found that H3K9ac and H3K27ac drove gene expression in EtOH-treated WT animals genome-wide (FIG. 14A-14B). However, in line with the ChIP-seq data, this response was blunted in ACSS2 KD mice (FIG. 14C-14D). Functional analysis of genes that were both hyperacetylated and induced by EtOH in an ACSS2-dependent manner included enrichment in genes with functions in protein binding, cell junction, postsynaptic density, and response to drug (FIG. 14E-14F). Together, these in vivo findings show that alcohol administration leads to increased histone acetylation and transcriptional activity in the dHPC in an ACSS2-dependent manner.

3. Ex Vivo Assay for Direct Effects of Exogenous Acetate on Gene Expression

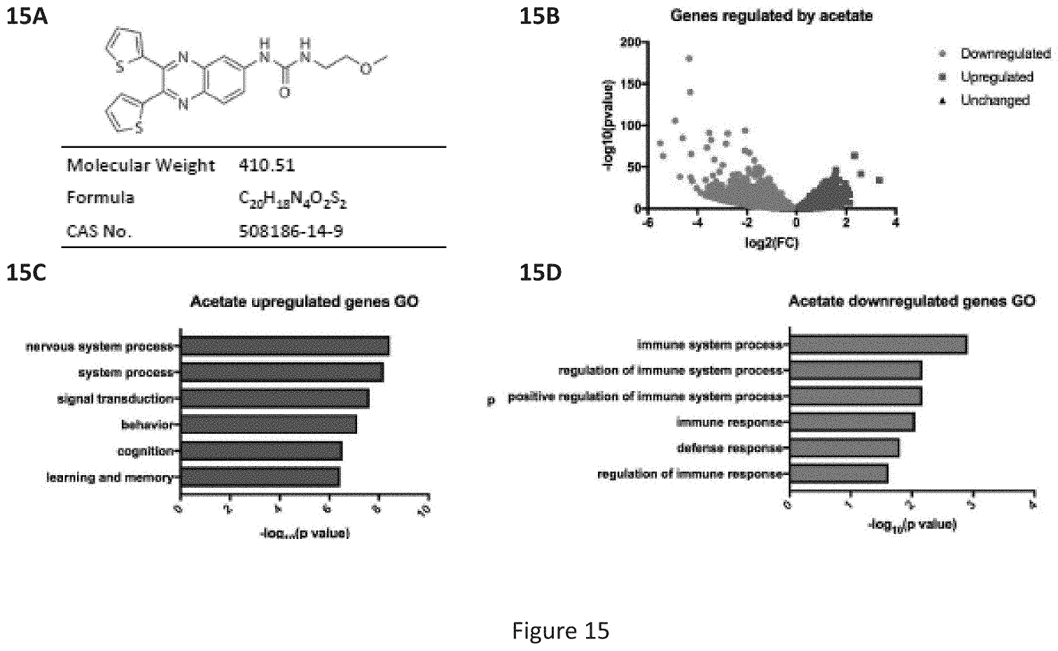

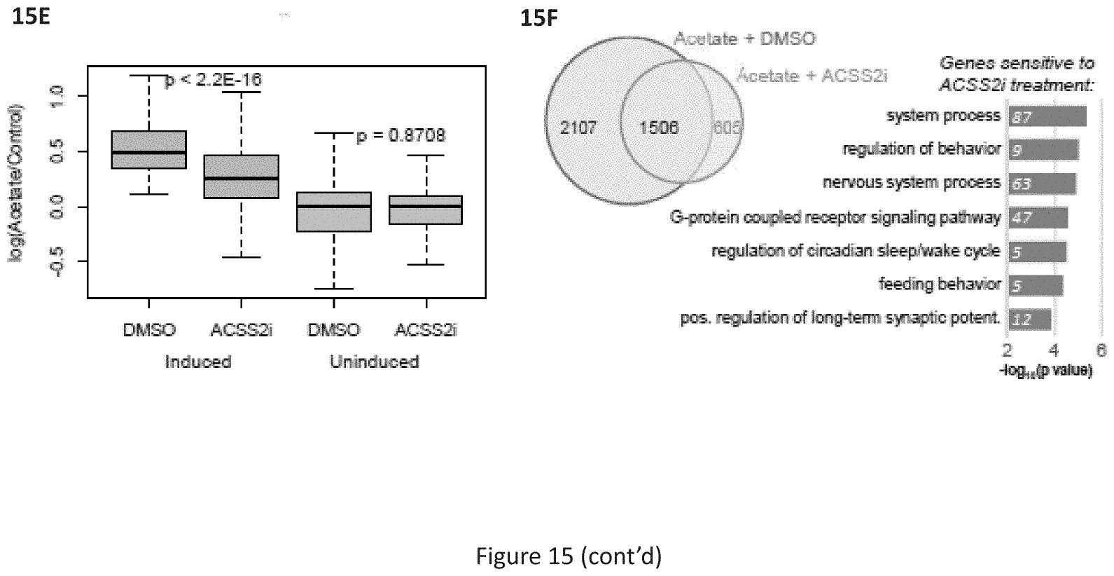

Alcohol and acetate have pleiotropic effects on brain circuitry and metabolism. Utilizing isolated mouse primary hippocampal neurons, the transcriptional response to supraphysiological levels of acetate (cells were cultured for one week after isolation and subsequently treated with 5 mM acetate for 24 hours) that mimics exogenous acetate influx during alcohol intake was investigated. Further, to determine the specific role of ACSS2 in transcriptional responses to acetate, a highly specific small molecule inhibitor of ACSS2 (ACSS2i ADG-205: C20H18N4O2S2, FIG. 15A) was employed.

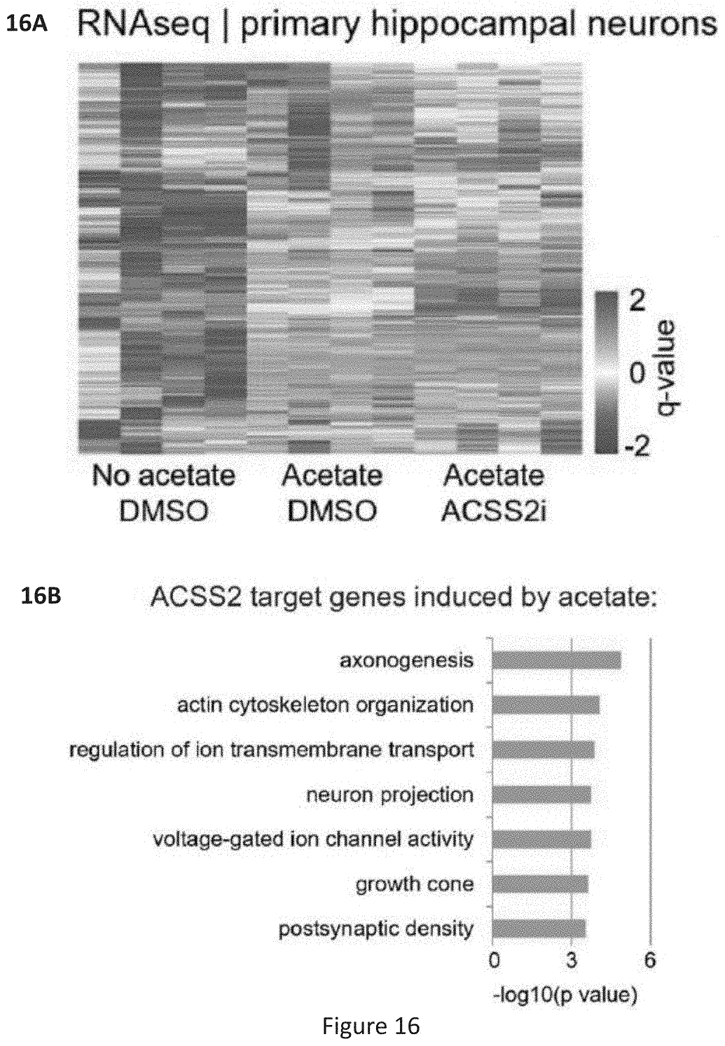

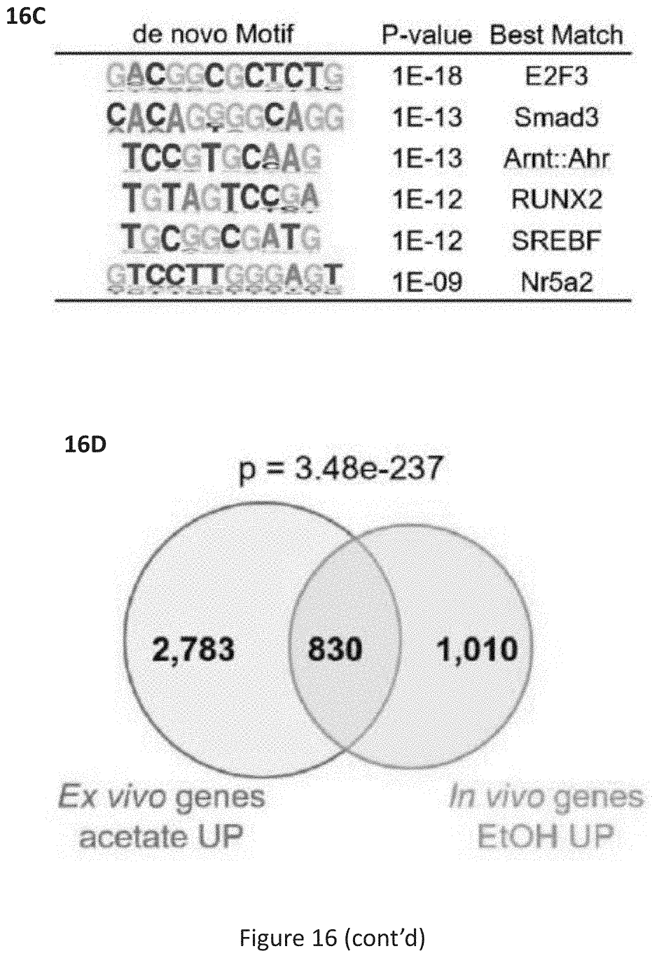

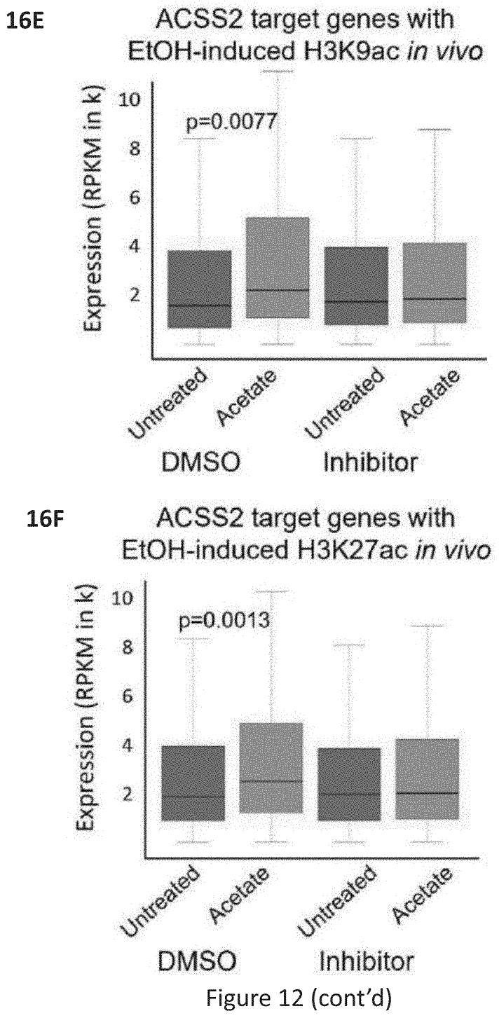

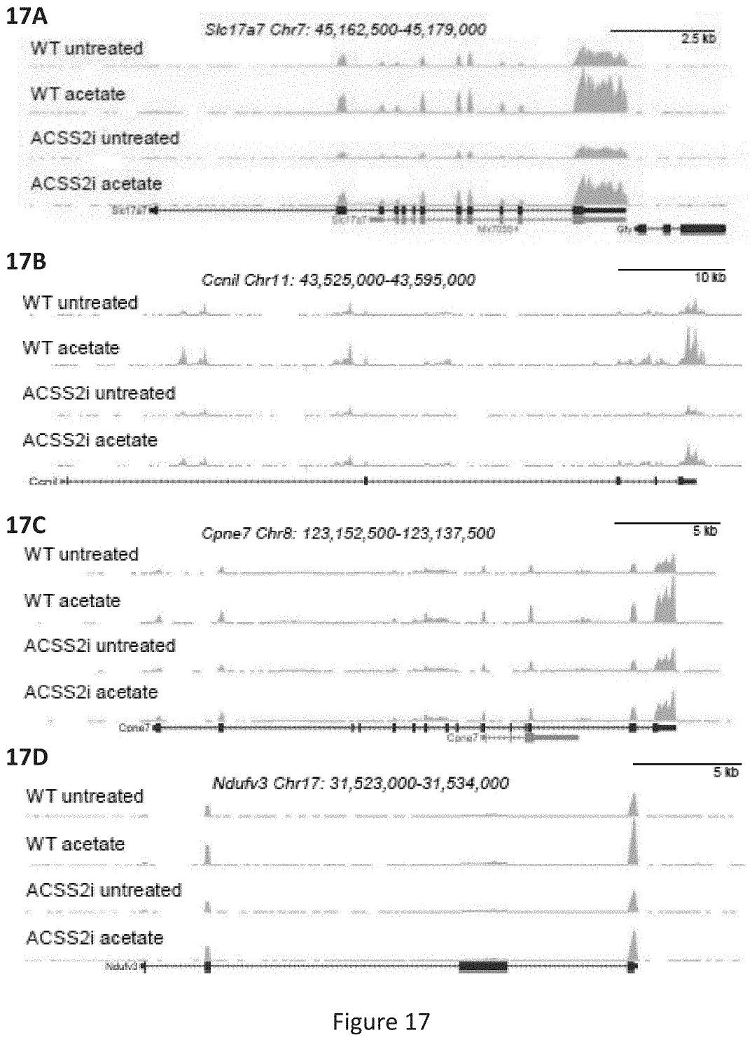

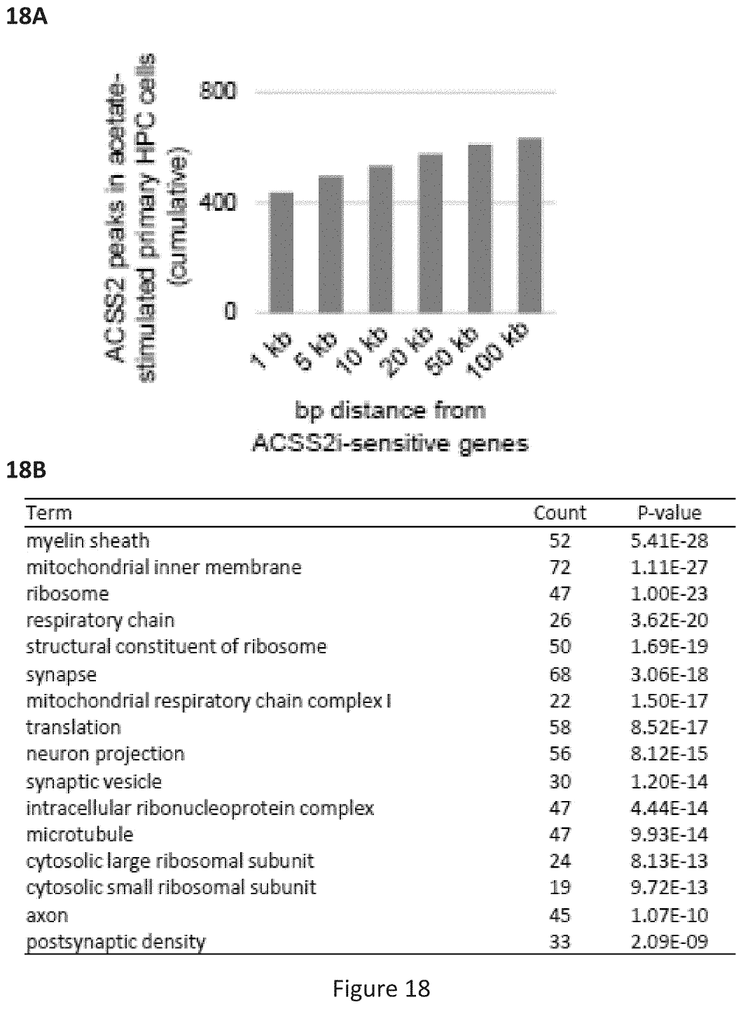

In primary hippocampal neurons, acetate supplementation induced 3613 genes (FIG. 16A, FIG. 15B) that were, via Gene Ontology (GO) term analysis, involved in nervous system processes, including signal transduction and learning and memory (FIG. 15C). In contrast, acetate treatment resulted in down regulation of genes involved in immune system processes (FIG. 15D). In the presence of the ACSS2i, 2107 of the acetate-induced genes failed to become upregulated (FIG. 15F), indicating that acetate-induced transcription relies heavily on the catalytic activity of ACSS2. Importantly, acetate-induced genes were not regulated by ACSS2i treatment in the absence of acetate (uninduced right boxes in FIG. 15E). GO analysis of ACSS2i-sensitive upregulated genes showed enrichment for nervous system processes, behavior, and learning and memory (FIG. 15F) and specific genes showed ACSS2i sensitivity (FIG. 17A-17D). For example, Scl17a7 was upregulated upon acetate treatment in WT hippocampus cells but induction was diminished when ACSS2 was inhibited (FIG. 17A). Slc17a7 encodes vesicular glutamate receptor 1 (Vglut1), implicated in hippocampal synaptic plasticity, addiction and alcohol use. In addition, impaired DNA methylation of Ccnjl (Cyclin J-like) has been linked to prenatal alcohol exposure and FASD (FIG. 17B). Further analysis revealed that the ACSS2i-sensitive and acetate-upregulated genes were also bound by hippocampal ACSS2 (our previous ChIP-seq), and binding was promotor-proximal at baseline without any direct behavioral stimulation in vivo (FIG. 18A). GO analysis linked these ACSS2 target genes to intricate plasticity-related mechanisms involving axonogenesis and voltage-gated ion channel activity (FIG. 16B). Correspondingly, motif analysis of ACSS2-targeted, acetate-induced, and ACSS2i-sensitive genes implicated the involvement of neuronal transcription factors—including E2F3 and NR5A2 (FIG. 16C)—linked to neurodifferentiation and the regulation of behavior by drugs of abuse.

Notably, there was substantial overlap of genes that were upregulated by alcohol in vivo in dorsal hippocampus and genes that were induced by acetate ex vivo (RNA-seq found 830 alcohol-responsive hippocampal genes to overlap with the ex vivo differentially expressed genes; FIG. 16D), supporting the translational validity of the ex vivo model. GO analysis for these overlapping genes indicated enrichment of genes related to to neuronal plasticity, including synapse, neuron projection, and axons, but also ribosomal and mitochondrial functions (FIG. 18B). Notably, a previously published microarray data set of in vivo alcohol-regulated hippocampal genes also showed substantial overlap with the described list of ex vivo acetate-induced genes (38% of 214 alcohol-responsive hippocampal genes in the microarray). Next, starting from our in vivo data in a complementary analysis, ACSS2 target genes with alcohol-induced H3K9ac in hippocampus in vivo were also upregulated by acetate treatment of hippocampal neurons ex vivo, and that ACSS2i blocks this gene induction (FIG. 16E). The equivalent relationship existed for hippocampal genes with alcohol-induced H3K27ac in vivo, which failed to be induced by acetate ex vivo in the presence of ACSS2i (FIG. 16F).

Together, these findings suggest that ACSS2 may play a role in alcohol-related learning via coordinating alcohol-induced histone acetylation and gene expression.

4. Ethanol-Mediated Conditioned Place Preference

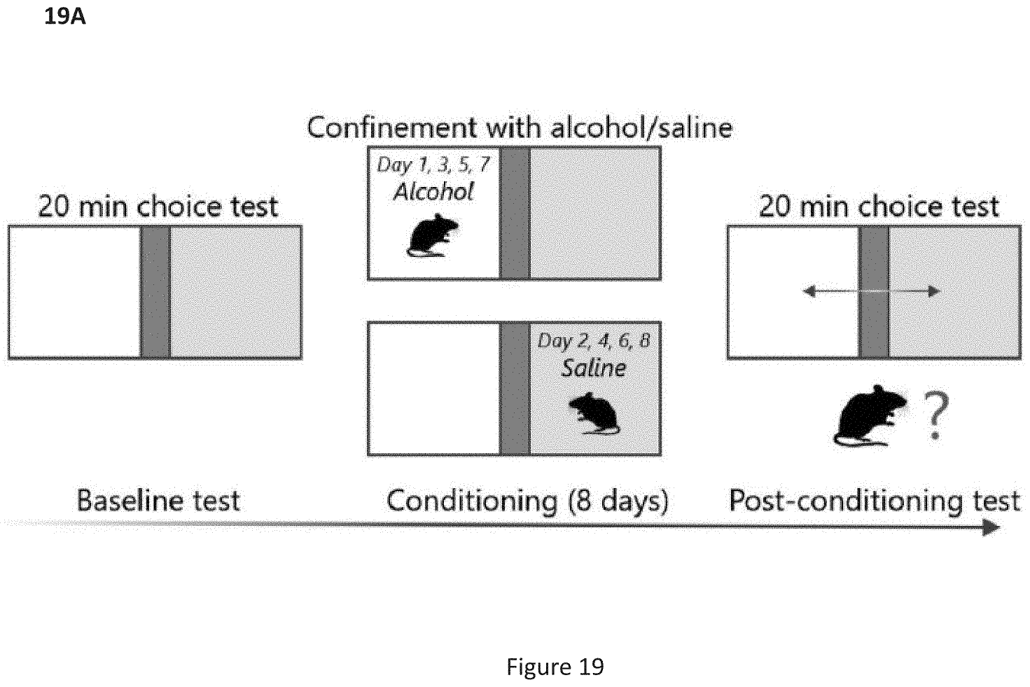

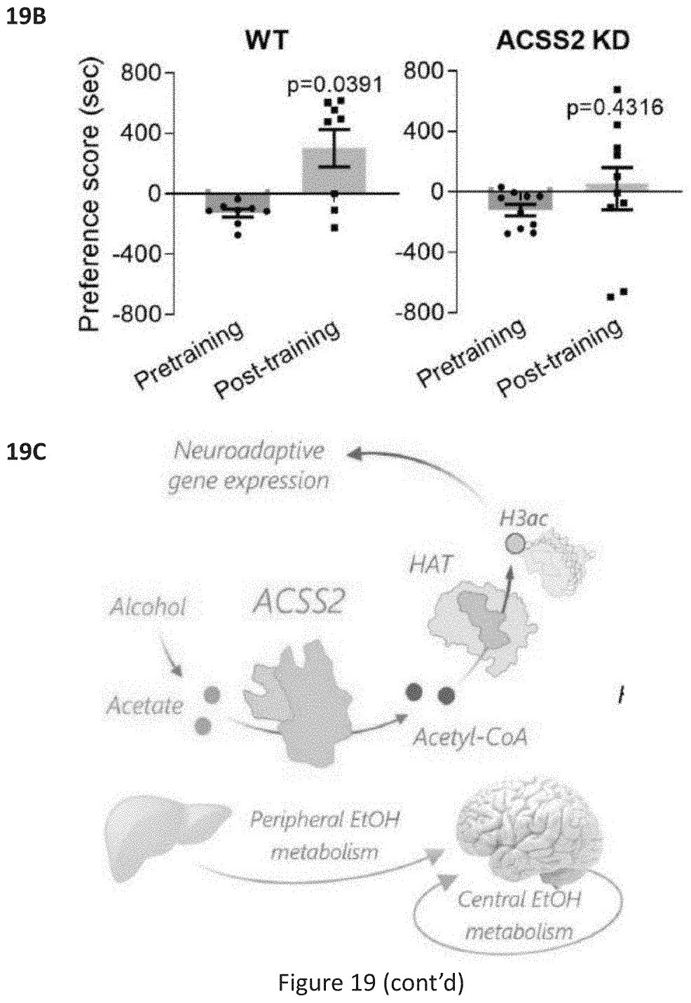

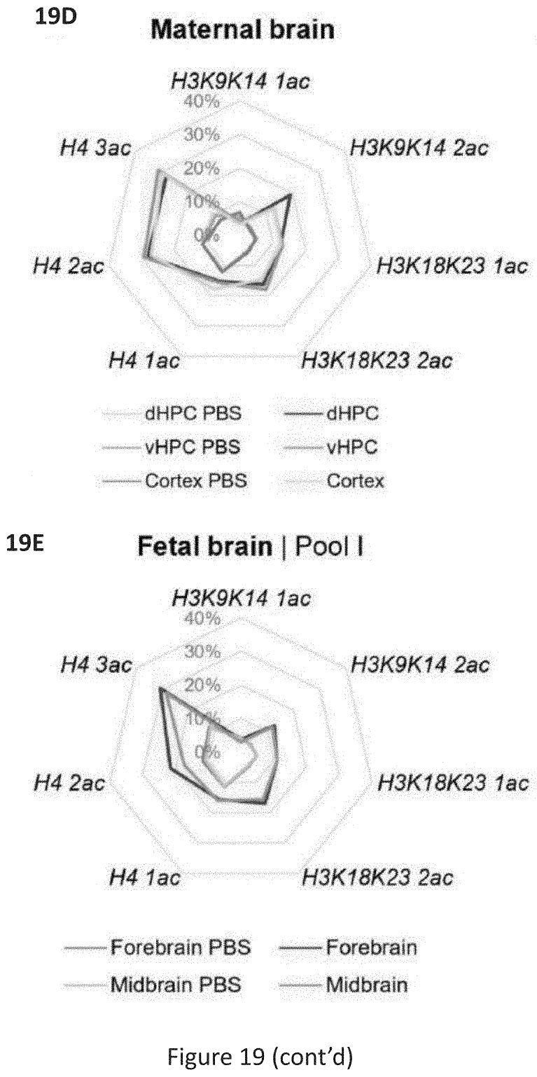

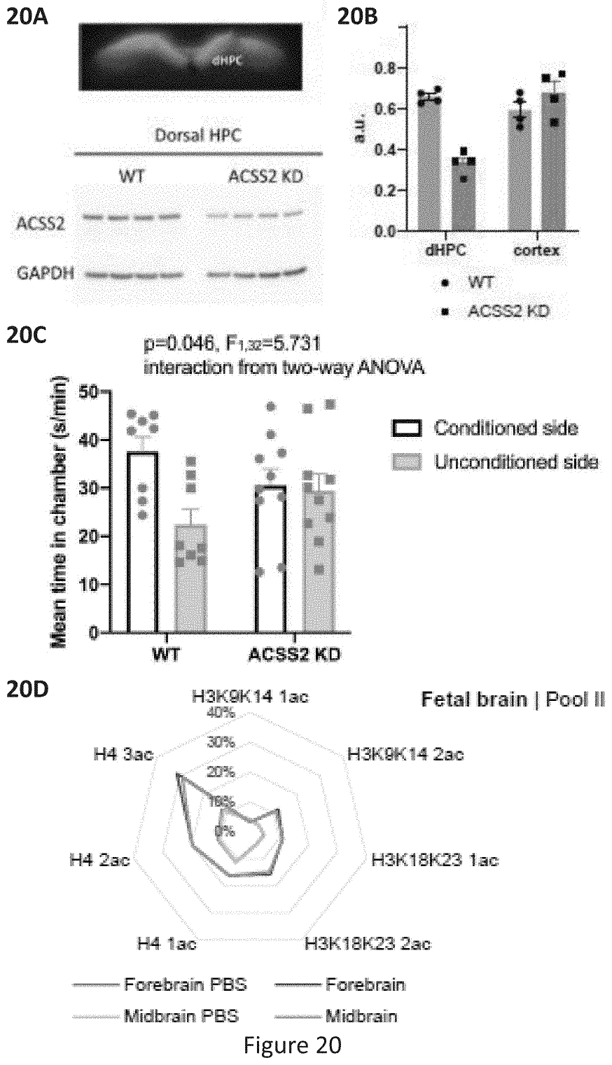

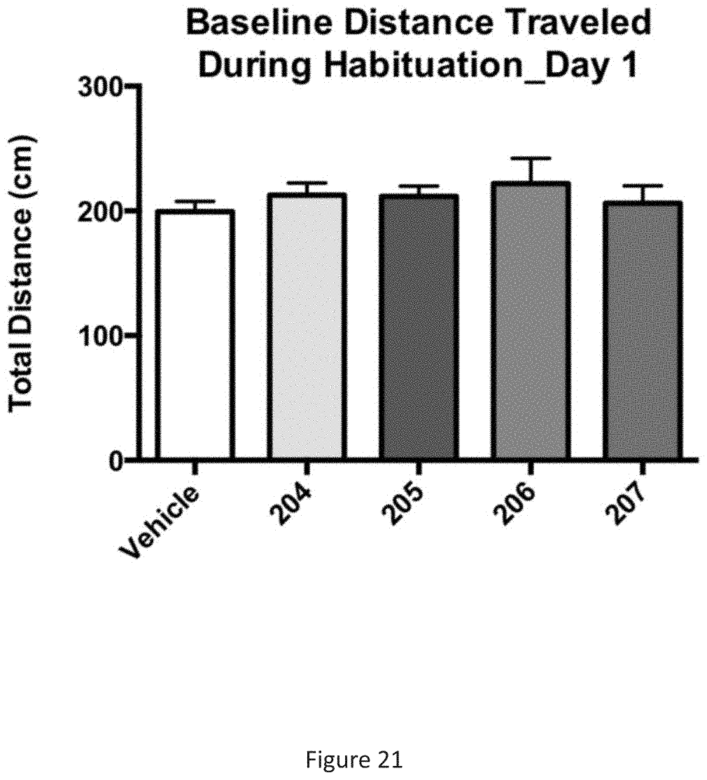

Ethanol-mediated conditioned place preference (CPP), which has been previously used to assess ethanol-associated learning. In this paradigm, animals are exposed to neutral and rewarding stimuli in distinct spatial compartments, distinguished by environmental cues. After conditioning, CPP is measured by allowing the animals free access to either compartment and measuring time spent in the reward-associated chamber (FIG. 19A). To assess place preference learning, mean time spent in the conditioned and unconditioned chambers was calculated (FIG. 20C), as well as a CPP score, which is defined as the difference between time spent in the conditioned versus the unconditioned chamber (FIG. 19B). WT mice was shown to spend increased time in the compartment in which ethanol was delivered during training (Wilcoxon, p=0.0391, FIG. 19B). Importantly, acquisition of CPP depends on dorsal HPC (dHPC) spatial memory formation, and, accordingly, dorsal HPC lesions disrupt place conditioning²¹. To test the importance of ACSS2 in the dHPC, GFP-expressing lentivirus mediated shRNA knock down was used to reduce the protein level of ACSS2 (n=10) compared to control shRNA (n=8; FIGS. 20A-20B). A significant main effect of the conditioning subgroup was observed (p=0.0227; F_{1.32}=5.731; main effect of “training” from 2-way ANOVA across the 4 groups), showing that the ethanol-induced CPP procedure was successful. Importantly, a significant treatment× conditioning subgroup interaction was shown (p=0.0462; F_{1.32}=4.303; interaction from 2-way ANOVA across the 4 groups), indicating that the treatment variable (i.e. the dorsal hippocampal ACSS2 KD) significantly reduced the expression of CPP. Strikingly, ethanol-associated CPP was abolished in ACSS2 KD (dHPC) mice (Wilcoxon, p=0.4316, FIG. 19B) indicating that ethanol-related associative memory formation requires ACSS2.

Taken together, the ex vivo and in vivo molecular data, along with the behavioral findings, show that ACSS2 is required for heavy labeled acetate incorporation into acetylated histones in the dorsal HPC, which facilitates memory-related gene expression and alcohol-related associative learning (FIG. 19C).

5. Effects on Gestating Fetus and Development

Alcohol exposure not only disrupts epigenetic and transcriptional processes in the adult brain but is also linked to epigenetic dysregulation in the gestating fetus. In utero, alcohol is an environmental teratogen that affects neuro-developmental gene expression and can elicit numerous alcohol-associated postnatal disease phenotypes that together are categorized as fetal alcohol spectrum disorder (FASD). Recent investigations of alcohol-mediated epigenetic changes in utero have implicated altered histone acetylation in FASD, but the underlying mechanisms are unknown.

Alcohol affects in dynamic histone acetylation in utero in the developing fetal mid- and forebrain (E18.5) was investigated. Fetal brain MS showed that ‘binge drinking-like’ alcohol exposure—parallel to maternal labeling of neuronal histone acetylation—resulted in deposition of alcohol-derived acetyl-groups onto histones in fetal fore- and midbrain in early neural development (FIGS. 19E and 20D), indicating an unanticipated potential mechanism for FASD etiology.

### Example 7: Animal Behavioral Models

A. Fear Conditioning in Rats

### Animal Description

Species: Rat; Strain: Male Sprague-Dawley (CD-SD strain 001; Charles River Labs); Age or weight: 7 to 9 weeks, approximately 250 grams

Randomization: Animals are assigned randomly to treatment groups

Blinding of Study: The study is not blinded

Acclimation/Conditioning: Not less than 5 days; handled 3 days prior to study

Housing: Rats are housed on a 12 hr light/dark cycle (lights on 7:00 AM); No more than 2 rats per cage depending on size; Rats are housed without enrichment; Ventilated cage rack system

Diet: Standard rodent chow and water ad libitum

Route(s) of administration: IP

Formulation(s): 5% DMSO in 0.5% methylcellulose

Dose Levels: 4 mg/kg total; distributed into 2 injections of 2 mg/kg

Dose Frequency: Twice; once prior to shock cycle and once right after last shock
1^{st} Injection—5 minutes before being placed into fear conditioning chamber
2^{nd} Injection—30 minutes after coming out of fear conditioning chamber (alternatively 30 min after last shock)
Study duration: 3 days

Pretreatment time (up to 2 hrs):
Standard Protocol: on Day 1 dosed before Acquisition AND right after Acquisition (provided necessary transition time); Note that rats are in the fear conditioning chambers for a total of 10 minutes for a 5 tone-shock pairing procedure. The first 3 minutes are habituation prior to the 1st tone-shock pairing

Number of Groups: 5

Number of animals per group: 12

Total number of animals: 60

Table 16 demonstrates the study design. A dose of 2 mg/kg before shock and 2 mg/kg after the shock were administered for a total of 4 mg/kg.

TABLE 16
Study Design: Standard Fear Conditioning Procedure

| | Dose level & | Days of | Group | Evaluations/ |
|---|---|---|---|---|
| Treatment | Route | dosing | Size | Endpoints |
| Vehicle | 0 IP | 1 | 12 | Freezing |
| ADG I-204 | 2 mg/kg IP | | | behavior, |
| ADG I-205 | 2 mg/kg IP | | | days 1-3 |
| ADG I-206 | 2 mg/kg IP | | | |
| ADG I-207 | 2 mg/kg IP | | | |

Table 17 shows the summary of behavior procedure

TABLE 17
Study Design: Standard Fear Conditioning
Procedure (US is 1 mA foot shock)

| | |
|---|---|
| Treatment | Day |

| Group | 1* | 2 | 3 |
|---|---|---|---|
| Vehicle | A: CS-US; Veh | A: CS (context) | B: CS (Cue) |
| ADG I-204 | A: CS-US; 204 | A: CS | B: CS |
| ADG I-205 | A: CS-US; 205 | A: CS | B: CS |
| ADG I-206 | A: CS-US; 206 | A: CS | B: CS |
| ADG I-207 | A: CS-US: 207 | A: CS | B: CS |

### Experimental Method

Rats are handled for 3 consecutive days for ˜2 minutes each day prior to the experiment. On day 1 of the experiments, animals are acclimated to the procedure room for at least 30 minutes prior to the start of experimental sessions each day. Fear conditioning is conducted in automated chambers built by Kinder Scientific (Poway, Calif.), which detects movement with infrared beams.

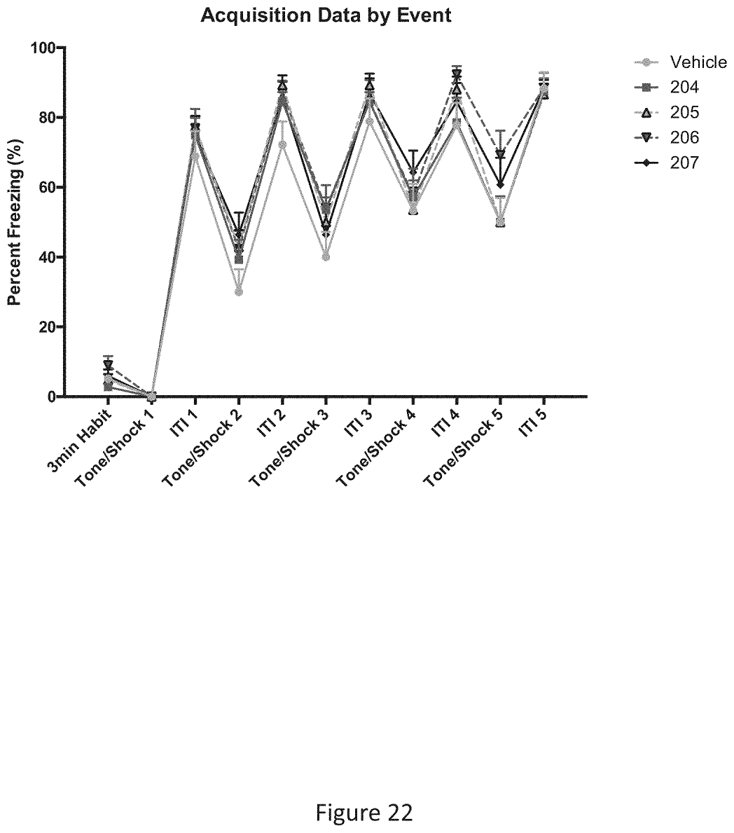

Day 1—Conditioning (Acquisition)—animals are placed in the chambers and presented a training session (Context A). An almond scent is present under the grid floor during the entire session. The training consists of a 3-minute habituation followed by a 20-second, 80 dB tone; during the last 3 seconds of the tone animals receive a 1 mA foot shock. This procedure is repeated four times at 1-minute intervals for a total of five paired presentations of tone and shock. See FIG. 22. Freezing behavior (immobility) is recorded in 10-second intervals during the session. Percent baseline freezing behavior is determined in the 3-minute habituation period.

To examine the effect of test compound on memory consolidation animals are dosed with vehicle or test compound before and after acquisition training on day 1.

Rats are in the fear conditioning chambers for a total of 10 minutes for a 5 tone-shock pairing procedure. The first test compound dose is administered 5 minutes prior to habituation.

Before the first 3 minutes of acquisition training—consisting of habituation to the box prior to the onset of the tone-shock pairing. The test compound pretreatment time of 5 min is determined prior to placing animals in the fear conditioning chambers. After acquisition training, rats are placed back into their home cages for 30 minutes prior to receiving the second test compound injection. Rats receive the second injection 30 minutes after coming out of the fear conditioning chamber, or alternatively 30 minutes after receiving the last shock. Table 17 depicts a sample timing of events for Day 1 Acquisition.

TABLE 17
Sample Timing - Day 1 Acquisition
5 Shock Pairings

| | | | | | Dose time |
|---|---|---|---|---|---|
| | Dose | Time in | Time of las | Time out | 30 min post |
| Rat # | Time | box | tone/shock | of box | treatment |
| 1 | 9:25 | 9:30 | 9:39 | 9:40 | 10:10 |
| 2 | 9:25 | 9:30 | 9:39 | 9:40 | 10:10 |
| 3 | 9:25 | 9:30 | 9:39 | 9:40 | 10:10 |
| 4 | 9:25 | 9:30 | 9:39 | 9:40 | 10:10 |

Day 1 Acquisition

The rats were provided 3 minutes habituation. Acquisition of fear consisted of 5 tone-shock pairings; 60 sec 1T1, 20 sec tone; 1 mA shock during last 3 sec. Data is recorded in 10 see epochs. The rats were administered drug (or vehicle) 5 min before acquisition and 30 min after acquisition session (n=13-15). Vehicle—5% DMSO: 95% MC; 2 IP injections 204, 205, 206, 207-8 mg/kg total; 2×4 mg/kg.

Day 2—Contextual Memory Test

Animals are placed in the chambers with the same almond scent (Context A). The session lasts 8 minutes and freezing behavior in response to context is recorded in 10-second intervals during the session. Contextual memory is determined by the percent of freezing behavior during the 8 minutes.

Day 3—Cued Memory Test

Animals are placed in the chambers with a different (lemon) scent and black Plexiglas floor over the grid floor (Context B). Freezing behavior in response to an altered context is recorded for 2 minutes in 10-second intervals. Then the 80 dB tone is presented for 8 minutes and immobility is recorded in 10-second intervals to measure freezing response to the tone cue.

Rat Fear Conditioning Contextual & Cued Freezing by Minute

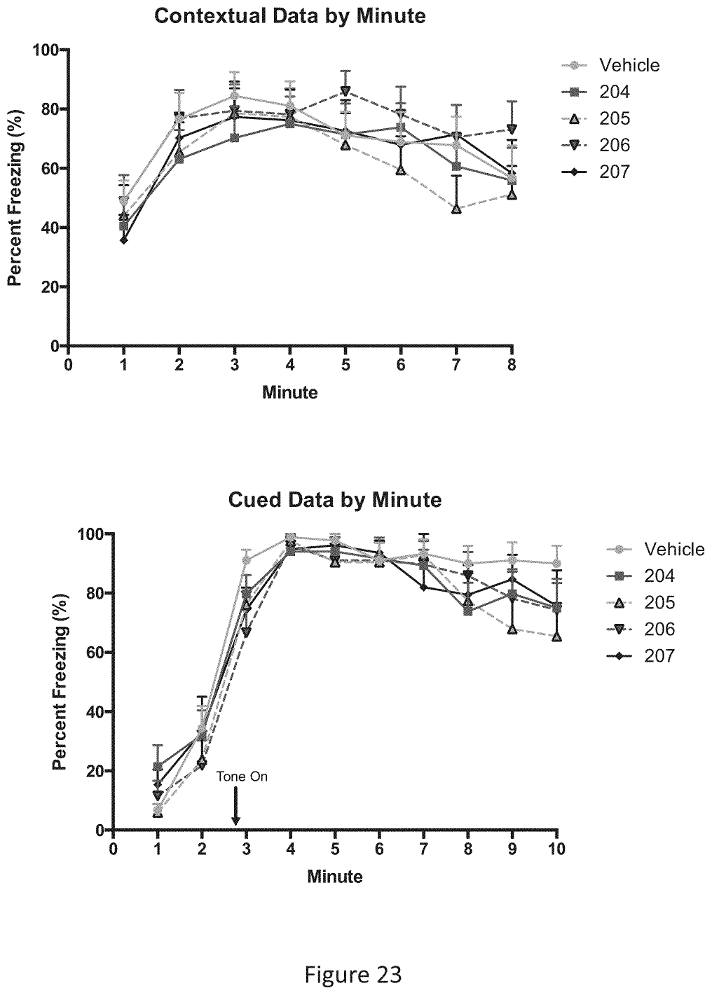

The Cue Memory test was performed. FIG. 23 shows that there is a significant reduced freezing in the EPV018 (ADG 205) cohort (above study, combined with a second study with 205).

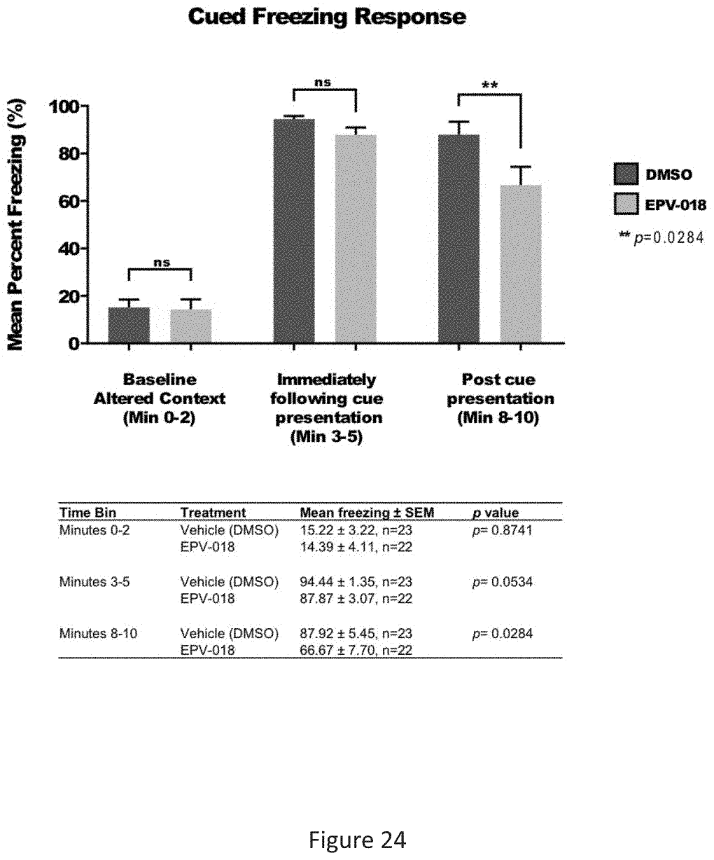

FIG. 24 demonstrates the cued freezing response details, including statistical analysis, for EPV018 (ADG 205).

FIG. 22 demonstrates that compared to DMSO treated animals, animals treated with ADG-205c have very little preference for one object. The conditions were blind as it relates to the injection (DMSO versus ADG-205c) and the locations of objects (those in the same location versus novel location). An online stopwatch interface was used that allowed use 3 simultaneous timer, one for each object. Each timer was stopped individually as the mouse interacted with a given object. This scoring demonstrates the recall phase. Some mice remained stationary for substantial amounts of time relative to the others—some appearing apprehensive huddled in a corner, some spending time grooming.

### Example 8: Fear Reconsolidation

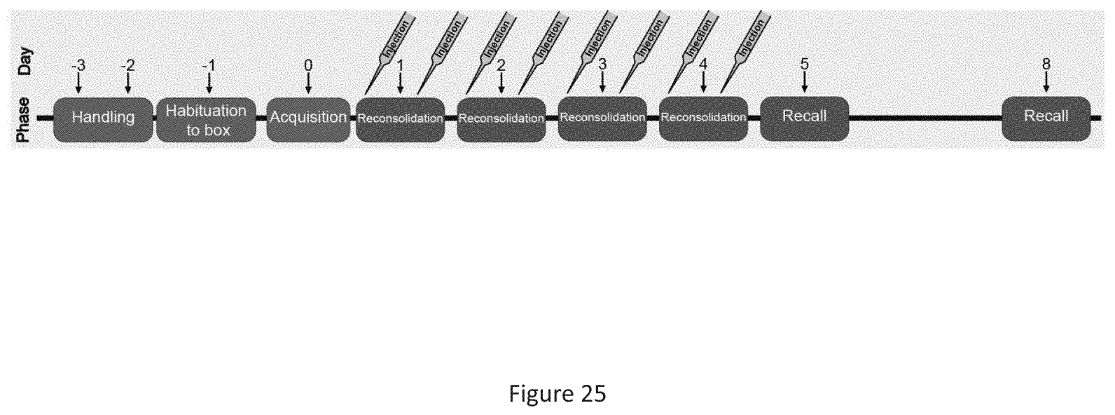

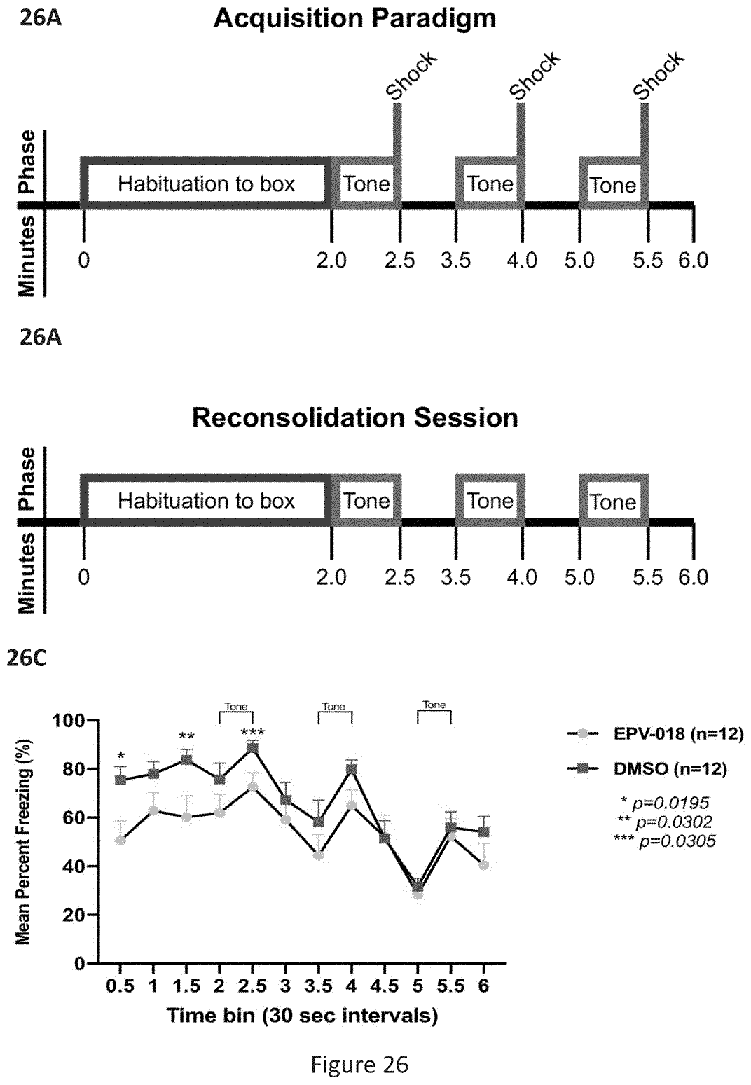

Table 18 and FIG. 25 demonstrate the experimental conditions mice were subjected to.

TABLE 18

| Day | Day | Experimental Phase |
|---|---|---|
| Monday | −3 | Handle |
| Tuesday | −2 | Handle |
| Wednesday | −1 | Mouse in chamber for 3 minutes, no CS, no US |
| Thursday | 0 | Acquisition - 3 CS-US pairings ending in 2 second, |
| | | 1 mA shock |
| Friday | 1 | Reconsolidation - injections before and after with |
| | | drug or DMSO |
| Saturday | 2 | Reconsolidation - injections before and after with |
| | | drug or DMSO |
| Sunday | 3 | Reconsolidation - injections before and after with |
| | | drug or DMSO |
| Monday | 4 | Reconsolidation - injections before and after with |
| | | drug or DMSO |
| Tuesday | 5 | Final Recall - no injection |
| Wednesday | 6 | |
| Thursday | 7 | |
| Friday | 8 | Additional recall date |

Acquisition Protocol

3 CS-US pairings were performed: tone is 30 seconds long co-terminating with 2 second, 1 mA shock. No drug or vehicle was administered.

Subsequent to the acquisition, and following the schedule provided, above, recall and reconsolidation sessions ensued. The cage was altered in context (floor board, walls, vanilla scent). The same paradigm as acquisition was used for reconsolidation and recall exposures but no shock was delivered. The following procedure was followed: (1) 2 min habituation to box; (2) 30 sec tones; (3) 1 min it is.

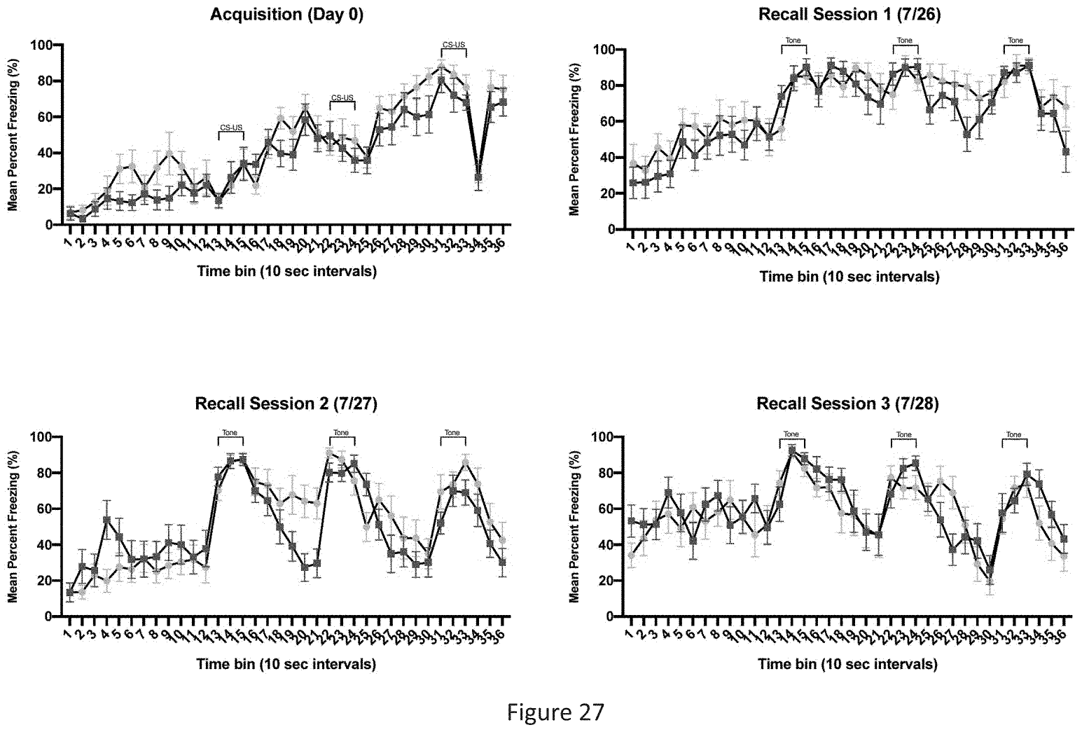

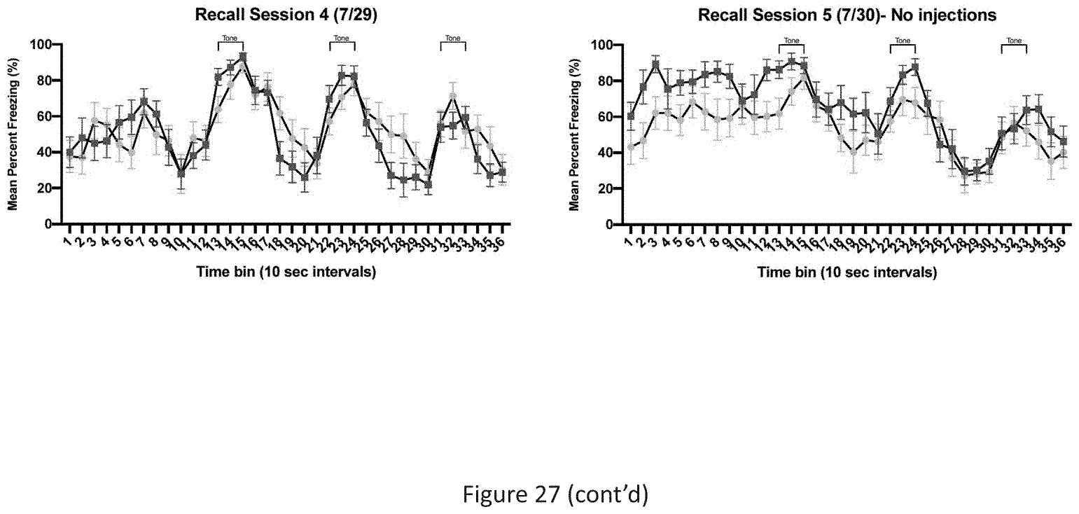

Injections of ADG-205c or DMSO at dosage of 2 mg/kg are administered at the following time points: Immediately before reconsolidation/recall; and then 30 minutes following reconsolidation/recall. The data was binned in 10 sec intervals, and shown in FIG. 27. After 4 sessions of dosed cue-recall sessions, the 205 cohort showed an observable reduced fear response.

### Example 9: Cocaine

The data presented herein demonstrates that ACSS2 inhibition could be a novel therapeutic avenue to target the encoding and maintenance of memories related to drug-associated environmental cues.

Cocaine-mediated conditioned place preference (CPP), which has been previously used to assess cocaine-associated learning was used. In this paradigm, animals are exposed to neutral and rewarding stimuli in distinct spatial compartments, distinguished by environmental cues. After conditioning, CPP is measured by allowing the animals free access to either compartment and measuring time spent in the reward-associated chamber. To assess place preference learning, mean time spent in the conditioned and unconditioned chambers was calculated (Cunningham et al, Nature Protocols 2006).

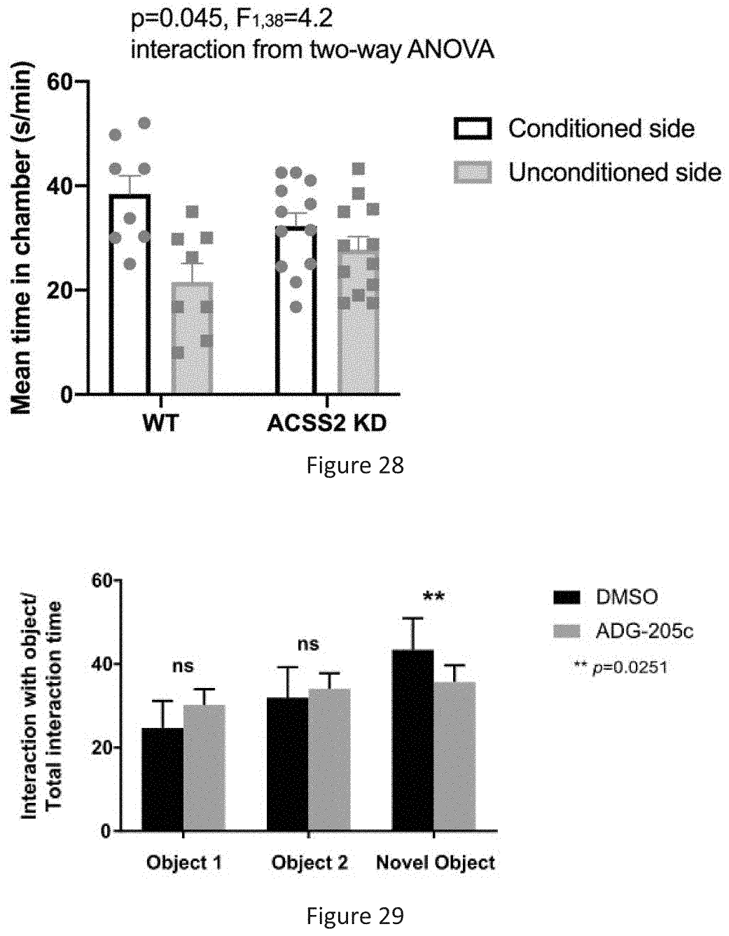

Importantly, acquisition of CPP depends on dorsal HPC (dHPC) spatial memory formation, and, accordingly, dorsal HPC lesions disrupt place conditioning. To test the importance of ACSS2 in the dHPC, GFP-expressing lentivirus mediated shRNA knock down was used to reduce the protein level of ACSS2 (n=12) compared to control shRNA (n=8). A significant main effect of the conditioning subgroup was observed (p=0.001; F1.36=12; main effect of “training” from 2-way ANOVA across the 4 groups) showing that the cocaine-induced CPP procedure was successful. Importantly, a significant treatment×conditioning subgroup interaction was also observed (p=0.0456; F_{1.36}=4.2; interaction from 2-way ANOVA across the 4 groups), indicating that the treatment variable (i.e. the dorsal hippocampal ACSS2 KD) significantly reduced the expression of CPP (FIG. 28). These results indicate that cocaine-related associative memory formation requires ACSS2.

### Example 10: Treating PTSD Patients

As shown elsewhere herein, ADG2-205 blocks acetyl-CoA synthetase (ACSS2) which regulates histone acetylation and hippocampal memory. In animal models, ACSS2 knock-down impairs long-term spatial memory and inhibits the upregulation of memory-related neuronal genes. In animal models, administration of ACSS2 inhibitor affects reconsolidation of memories of toxic stimuli, leaving other memory functions and growth and development intact. Example 8 demonstrates the reconsolidation of memories of toxic stimuli in animal models.

ADG2-205 is used along with psychotherapy (augmented psychotherapy) to treat individuals with posttraumatic stress disorder (PTSD). Phase 1 is conducted in healthy volunteers to assess any safety issues and assess blood levels to target therapeutic dose levels seen in pre-clinical animal models. Phases 2 and 3 studies are done in patients with PTSD.

### Phase 1

### Single Ascending Dose (SAD) Study

Up to 8 dose levels are determined by pre-IND toxicity studies in animals. Healthy volunteers are dosed in a Phase 1 unit. There are 10 individuals/dose level: 8 drug and 2 placebo per dose level (N=40). Patients are observed in the Phase 1 unit for 24 hours post-dosing. Follow-up visits are scheduled for 7 and 30 days post dosing.

Safety labs are done at screening, pre-dosing and 24 hours, and at Day 7 and Day 30 follow-up visits. Electrocardiograms (ECGs) are done at screening, pre-dosing, at 2 hours, 8 hours, 24 hours post-dosing, and at Day 7 and Day 30 follow-up visits. Memory test is done of overall memory function (standard test for short and long term memory) at screening, pre-dosing, 2 hours, 8 hours and 24 hours post dosing as well as at Day 7 and Day 30 follow-up visits. Blood is drawn for drug levels at 30 minutes, 1 hour, 2 hours, then every 2 hours until 24 hours.

### Multiple Ascending Dose (MAD) Study

Healthy volunteers are dosed in a Phase 1 unit. There are 10 individuals/dose level: 8 drug and 2 placebo per dose level (N≥20). Patients are observed in the Phase 1 unit for 24 hours post-dosing on each study day. Subjects participate in 4 sessions each separated by 1 week. Safety labs are done at screening, pre-dosing and 24 hours post-dosing, and at Day 7 and Day follow-up 30 visits. Electrocardiograms (ECGs) are done at screening, pre-dosing, and 8 and 24 hours post dosing on each study day and at Day 7 and Day 30 follow-up visits Memory test of overall memory function (standard test for short and long term memory) is done at screening, pre-dosing, 8 hours and 24 hours post dosing on each study day and at Day 7 and Day 30 visit.

Treatment of Patients with PTSD

Participants undergo 90-minute preparatory sessions with a therapy team. Psychiatric medications are tapered by the study physician and discontinued at least 5 half-lives before ACCS2 inhibitor administration. Subjects are randomized to receive 1 of 2 dose levels of drug or placebo in 1:1:1 ratio. Subjects receive study drug at the assigned study dose level of ACSS2 or placebo at the beginning of 5 double-blind 1 hour experimental sessions 1 week apart from one another. Subjects follow with 5 CPT sessions or an alternate cognitive behavioral therapy 1 week apart from one another. Some subjects repeat 5× over 5 weeks. The CAPS-V score serves as the primary outcome measure. A more than 30% drop in CAPS-V total scores are used to define a clinically significant change in PTSD symptoms. Secondary outcome measures assesses memory (standard test for short and long term memory).

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A method for treating or preventing a neurological and cognitive disease or disorder, the method comprising administering a composition comprising a compound of Formula (1) to a subject in need thereof: 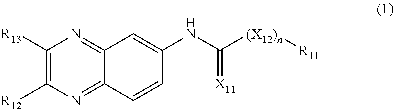
wherein, X₁₁ is selected from the group consisting of C(R₁₄)(R₁₅), O, S and NR₁₅;
each occurrence of X₁₂ is selected from the group consisting of C(R₁₄)(R₁₅), O, S and NR₁₅;
R₁₁ is selected from the group consisting of —C₁-C₂₅ alkyl, cycloalkyl, heterocyclyl, aryl, heteroaryl, and combinations thereof, wherein R₁₁ is optionally substituted;
R₁₂ and R₁₃ are each independently selected from the group consisting of hydrogen, —C₁-C₆ alkyl, —C₃-C₆ aryl, and —C₄-C₆ heteroaryl, wherein R₁₂ and R₁₃ are optionally substituted;
each occurrence of R₁₄ and R₁₅ is independently selected from the group consisting of hydrogen, halogen, —OH, and C₁-C₆ alkyl; and
n is an integer from 0-4.

2. The method of claim 1, wherein neurological and cognitive disease or disorder is selected from the group consisting of post-traumatic stress disorder (PTSD), depression, addiction or addiction-related disease or disorder, anxiety disorder, panic disorders, obsessive-compulsive disorder, and phobias.

3. The method of claim 1, wherein the neurological and cognitive disease or disorder is PTSD.

4. The method of claim 1, wherein addiction is alcoholism or cocaine addiction.

5. The method of claim 1, wherein the addiction-related disease or disorder is acute and/or chronic alcohol induced memory deficit.

6. The method of claim 1, wherein the compound of Formula (1) is a compound according to Formula (2): 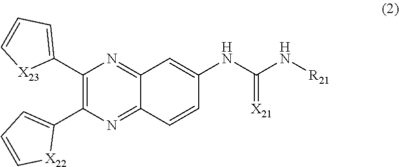
wherein, X₂₁ is O, or S;
X₂₂ and X₂₃ are each independently selected from the group consisting of NR₂₂, O, and S; and
R₂₁ is selected from the group consisting of —C₁-C₂₅ alkyl, cycloalkyl, heterocyclyl, aryl, heteroaryl, and combinations thereof, wherein R₁₁ is optionally substituted; and
each occurrence of R₂₂ is independently selected from the group consisting of hydrogen and C₁-C₆ alkyl.

7. The method of claim 1, wherein the compound of Formula (1) is a compound according to Formula (3): 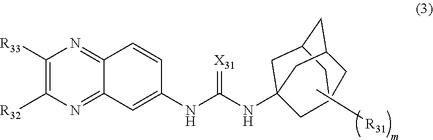
wherein, X₃₁ is selected from the group consisting of C(R₃₄)(R₃₅), O, S and NR₃₅;
each R₃₁ is independently hydrogen, —C₁-C₁₀ alkyl, halogen, —OH, or ═O or ═S formed by joining two R₃₁s,
R₃₂ and R₃₃ are each independently selected from the group consisting of hydrogen, —C₁-C₆ alkyl, —C₃-C₆ aryl, and —C₄-C₆ heteroaryl, wherein R₁₂ and R₁₃ are optionally substituted;
each occurrence of R₃₄ and R₃₅ is independently selected from the group consisting of hydrogen, halogen, —OH, and C₁-C₆ alkyl; and
m is an integer from 0-15.

8. The method of claim 1, wherein the compound is selected from the group consisting of

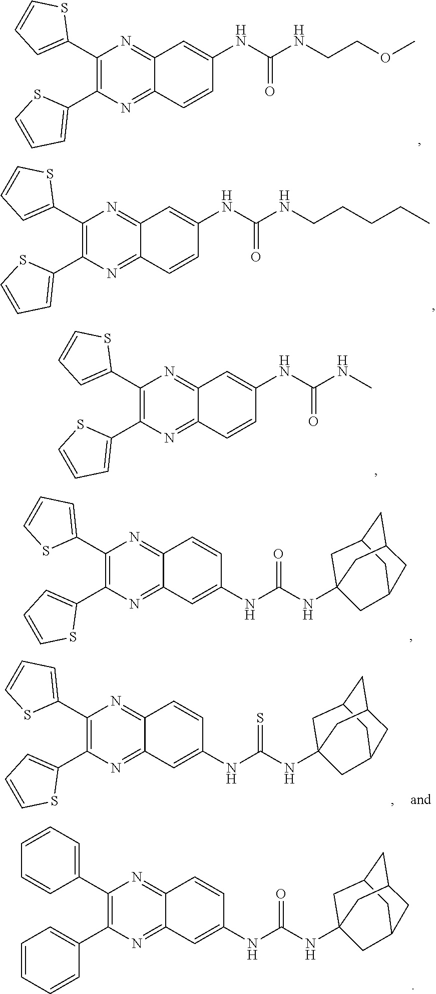

9. A compound according to Formula (1): 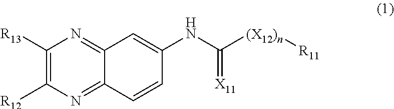
wherein, X₁₁ is selected from the group consisting of C(R₁₄)(R₁₅), O, S and NR₁₅;
each occurrence of X₁₂ is selected from the group consisting of C(R₁₄)(R₁₅), S and NR₁₅;
R₁₁ is selected from the group consisting of —C₁-C₂₅ alkyl, cycloalkyl, heterocyclyl, aryl, heteroaryl, and combinations thereof, wherein R₁₁ is optionally substituted;
R₁₂ and R₁₃ are each independently selected from the group consisting of hydrogen, —C₁-C₆ alkyl, —C₃-C₆ aryl, and —C₄-C₆ heteroaryl, wherein R₁₂ and R₁₃ are optionally substituted;
each occurrence of R₁₄ and R₁₅ is independently selected from the group consisting of hydrogen, halogen, —OH, and C₁-C₆ alkyl; and
n is an integer from 0-4.

10. The compound of claim 9, wherein the compound of Formula (1) is a compound according to Formula (2): 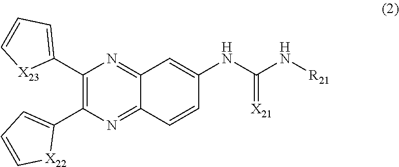
wherein, X₂₁ is O, or S;
X₂₂ and X₂₃ are each independently selected from the group consisting of NR₂₂, O, and S; and
R₂₁ is selected from the group consisting of —C₁-C₂₅ alkyl, cycloalkyl, heterocyclyl, aryl, heteroaryl, and combinations thereof, wherein R₁₁ is optionally substituted; and
each occurrence of R₂₂ is independently selected from the group consisting of hydrogen and C₁-C₆ alkyl.

11. The compound of claim 9, wherein the compound of Formula (1) is a compound according to Formula (3): 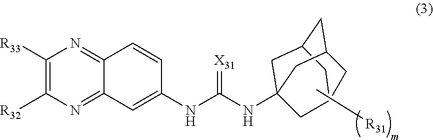
wherein, X₃₁ is selected from the group consisting of C(R₃₄)(R₃₅), O, S and NR₃₅;
each R₃₁ is independently hydrogen, —C₁-C₁₀ alkyl, halogen, —OH, or ═O or ═S formed by joining two R₃₁s,
R₃₂ and R₃₃ are each independently selected from the group consisting of hydrogen, —C₁-C₆ alkyl, —C₃-C₆ aryl, and —C₄-C₆ heteroaryl, wherein R₁₂ and R₁₃ may be optionally substituted;
each occurrence of R₃₄ and R₃₅ is independently selected from the group consisting of hydrogen, halogen, —OH, and C₁-C₆ alkyl; and
m is an integer from 0-15.

12. The compound of claim 9, wherein the compound is selected from the group consisting of

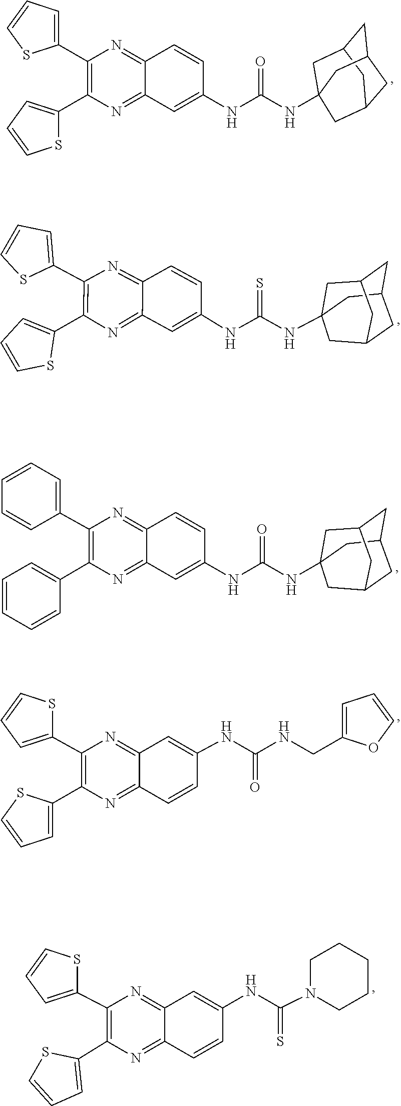 [SMILES: O=C(Nc1ccc2nc(-c3cccs3)c(-c3cccs3)nc2c1)NC12CC3CC(CC(C3)C1)C2 ; S=C(Nc1ccc2nc(-c3cccs3)c(-c3cccs3)nc2c1)NC12CC3CC(CC(C3)C1)C2 ; O=C(Nc1ccc2nc(-c3ccccc3)c(-c3ccccc3)nc2c1)NC12CC3CC(CC(C3)C1)C2 ; O=C(NCc1ccco1)Nc1ccc2nc(-c3cccs3)c(-c3cccs3)nc2c1 ; S=C(Nc1ccc2nc(-c3cccs3)c(-c3cccs3)nc2c1)N1CCCCC1]

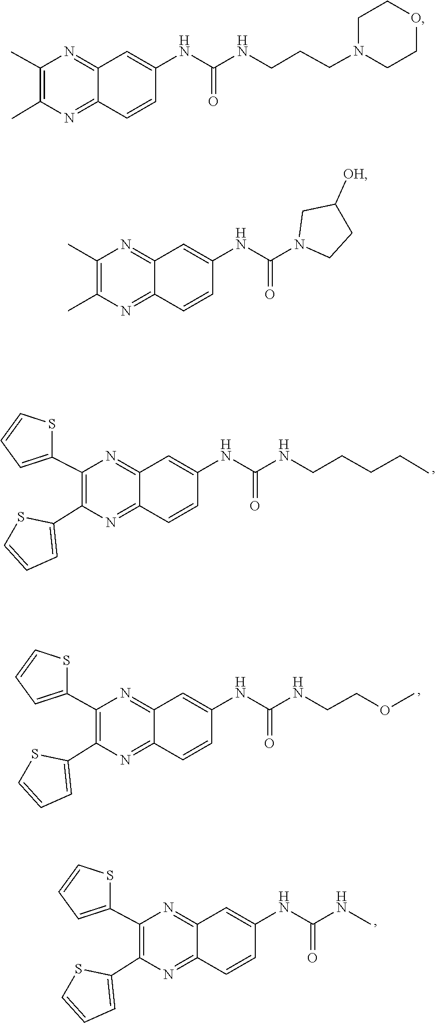 [SMILES: Cc1nc2ccc(NC(=O)NCCCN3CCOCC3)cc2nc1C ; Cc1nc2ccc(NC(=O)N3CCC(O)C3)cc2nc1C ; CCCCCNC(=O)Nc1ccc2nc(-c3cccs3)c(-c3cccs3)nc2c1 ; COCCNC(=O)Nc1ccc2nc(-c3cccs3)c(-c3cccs3)nc2c1 ; CNC(=O)Nc1ccc2nc(-c3cccs3)c(-c3cccs3)nc2c1]

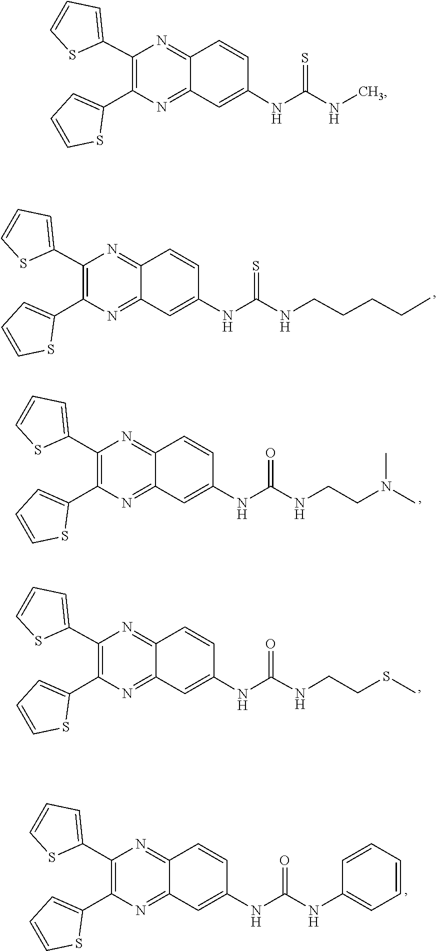

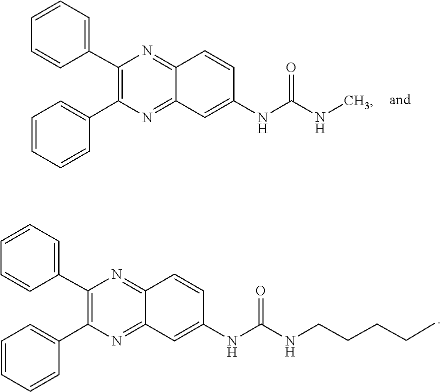 [SMILES: CNC(=O)Nc1ccc2nc(-c3ccccc3)c(-c3ccccc3)nc2c1 ; CCCCCNC(=O)Nc1ccc2nc(-c3ccccc3)c(-c3ccccc3)nc2c1]

13. A method for treating or preventing a neurological and cognitive disease or disorder in a subject in need thereof, comprising:
a) Treating the subject with the compound of claim 9 during trauma recall and memory reconsolidation; and
b) subsequently treating the subject with cognitive behavioral therapy.

14. The method of claim 13, wherein the treating step is repeated up to 12 times.

15. The method of claim 14, wherein the cognitive behavioral therapy is cognitive processing therapy.
